SEQUENCE LISTING (15 sequences)

SEQ ID NO: 1 - protein, 306 aa, Artificial sequence
  synthetic
SGPRKMAFPSGKVEGCMVQVTCGTTTLNGLWLDDTVYCPRHVICTAEDMLNPNYEDLLIRKSNHSPLVQAGNVQLRVIGHSMQNCLLRLKVDTSNPKTPKYKFVRIQPGQTPSVLACYNGSPSGVYQCAMRPNHTIKGSFLNGSCGSVGFNIDYDCVSFCYHHHMELFTGVHAGTDLEGKFYGPFVDRQTAQAAGTDTTITLNVLAWLYAAVINGDRWFLNRPTTTLNDFNLVAMKYNYEPLTQDHVDILGPLSAQTGIAVLDMCAALKELLQNGHNGRTILGSTILEDEFTPFDVVRQCSGVTFQ

SEQ ID NO: 2 - protein, 306 aa, Artificial sequence
  synthetic
SGLVKMSHPSGDVEACMVQVTCGSMTLNGLWLDNTVWCPRHVMCPADQLSDPNYDALLISHTNHSPSVQKHIGAPANLRVVGHAMQGTLLKLTVDVANPSTPAYTFTTVKPGAAPSVLACYNGRPTGTFTVVHRPNYTIKGSFLCGSCGSVGYTKEGSVINFCYHHQMELANGTHTGSAFDGTMYGAFHDKQVHQVQLTDKYCSVNVVAWLYAAILNGCAWFVKPNRTSVVSFNEWALANQPTEFVGTQSVDHLAVKTGVAIEQLLYAIQQLYTGFQGKQILGSTMLEDEFTPEDVNHQIMGVVHQ

SEQ ID NO: 3 - protein, 306 aa, Artificial sequence
  synthetic
SGPRKMAFPSGKVEGCMVQVTCGTTTLNGLWLDDVVYCPRHVICTSEDMLNPNYEDLLIRKSNHNPLVQAGNVQLRVIGHSMQNCVLKLKVDTANPKTPKYKFVRIQPGQTPSVLACYNGSPSGVYQCAMRPNPTIKGSFLNGSCGSVGFNIDYDCVSFCYMHHMELPTGVHAGTDLEGNFYGPFVDRQTAQAAGTDTTITVNVLAWLYAAVINGDRWFLNRPTTTLNDFNLVAMKYNYEPLTQDHVDILGPLSAQTGIAVLDMCASLKELLQNGHNGRTILGSALLEDEFTPFDVVRQCSGVTFQ

SEQ ID NO: 4 - protein, 302 aa, Artificial sequence
  synthetic
SGPRKMAFPSGKVEGCMVQVTCGTTTLNGLWLDDVVYCPRHVICTSEDMLNPNYEDLLIRKSNHNPLVQAGNVQLRVIGHSMQNCVLDTANPKTPKYKFVRIQPGQTPSVLACYNGSPSGVYQCAMRPNPTIKGSFLNGSCGSVGFNIDYDCVSFCYMHHMELPTGVHAGTDLBGNFYGPFVDRQTAQAAGTDTTITVNVLAWLYAAVINGDRWFLNRPTTTLNDFNLVAMKYNYEPLTQDHVDILGPLSAQTGIAVLDMCASLKELLQNGHNGRTILGSALLEDEFTPFDVVRQCSGVTFQ

SEQ ID NO: 5 - protein, 302 aa, Artificial sequence; identical to SEQ ID NO: 4
  synthetic

SEQ ID NO: 6 - protein, 304 aa, Artificial sequence
  synthetic
SGPRKMAFPSGKVEGCMVQVTCGTTTLNGLWLDDVVYCPRHVICTSEDMLNPNYEDLLIRKSNHNPLVQAGNVQLRVIGHSMQNCVLKLDTANPKTPKYKFVRIQPGQTPSVLACYNGSPSGVYQCAMRPNPTIKGSFLNGSCGSVGFNIDYDCVSFCYHHHMELPTGVHAGTDLEGNFYGPFVDRQTAQAAGTDTTITVNVLAWLYAAVINGDRWFLNRPTTTLNDFNLVAMKYNYEPLTQDHVDILGPLSAQTGIAVLDMCASLKELLQNGHNGRTILGSALLEDEFTPFDVVRQCSGVTFQ

SEQ ID NO: 7 - protein, 306 aa, Artificial sequence
  synthetic
SGPRKMAFPSGKVEGCMVQVTCGTTTLNGLWLDDVVYCPRHVICTSBDMLNPNYEDLLIRKSNHNPLVQAGNVQLRVIGHSMQNCVLKLKVDTANPKTPKYKFVRIQPGQTPSVLACYNGSPSGVYQCAMRPNPTIKGSFLNGSCGSVGFNIDYDCVSFCYMHHMELFTGVHAGTDLBGNFYGPFVDRQTAQAAGTDTTITVNVLAWLYAAVINGDRWFLNRPTTTLNDFNLVAMKYNYEPLTQDHVDILGPLSAQTGIAVLDMCASLKELLQNGHNGRTILGSALLEDEFTPFDVVRQCSGVTFQ

SEQ ID NO: 8 - protein, 306 aa, Artificial sequence
  synthetic
SGPRKMAFPSGKVEGCMVQVTCGTTTLNGLWLDDVVYCPRHVICTSEDMLNPNYEDLLIRKSNHNPLVQAGNVQLRVIGHSMQNCVLKLKVDTANPKTPKYKFVRIQPGQTPSVLACYNGSPSGVYQCAMRPNPTIKGSFLNGSCGSVGFNIDYDCVSFCYMHHMELPTGVHAGTDLBGNFYGPFVDRQTAQAAGTDTTITVNVLAWLYAAVINGDRWFLNRPTTTLNDFNLVAMKYNYEPLTQDHVDILGPLSAQTGIAVLDMCASLKELLQNGHNGRTILGSALLEDEFTPFDVVRQCSGVTFQ

SEQ ID NO: 9 - protein, 306 aa, Artificial sequence; identical to SEQ ID NO: 8
  synthetic

SEQ ID NO: 10 - protein, 306 aa, Artificial sequence; identical to SEQ ID NO: 8
  synthetic

SEQ ID NO: 11 - protein, 305 aa, Artificial sequence
  synthetic
SGPRKMAFPSGKVEGCMVVTCGTTTLNGLWLDDVVYCPRHVICTSEDMLNPNYEDLLIRKSNHNPLVQAGNVQLRVIGHSMQNCVLKLKVDTANPKTPKYKFVRIQPGQTPSVLACYNGSPSGVYQCAMRPNPTIKGSFLNGSCGSVGFNIDYDCVSFCYHHHMELFTGVHAGTDLBGNFYGPFVDRQTAQAAGTDTTITVNVLAWLYAAVINGDRWFLNRPTTTLNDFNLVAMKYNYEPLTQDHVDILGPLSAQTGIAVLDMCASLKELLQNGHNGRTILGSALLEDEFTPFDVVRQCSGVTFQ

SEQ ID NO: 12 - protein, 306 aa, Artificial sequence
  synthetic
SGPRKMAFPSGKVEGCMVQVTCGTTTLNGLWLDDVVYCPRHVICTSEDMLNPNYEDLLIRKSNHNPLVQAGNVQLRVIGHSMQNCVLKLKVDTANPKTPKYKFVRIQPGQTPSVLACYNGSPSGVYQCAMRPNPTIKGSFLNGSCGSVGFNIDYDCVSFCYMHHMELFTGVHAGTDLBGNFYGPFVDRQTAQAAGTDTTITVNVLAWLYAAVINGDRWFLNRPTTTLNDFNLVAMKYNYEPLTQDHVDILGPLSAQTGIAVLDMCASLKELLQNGHNGRTILGSALLEDEFTPFDVVRQCSGVTFQ

SEQ ID NO: 13 - protein, 304 aa, Artificial sequence
  synthetic
SGPRKMAFPSGKVEGCMVQVTCGTTTLNGLWLDDVVYCPRHVICTSEDMLNPNYEDLLIRKSNHNPLVQAGNVQLRVIGHSMQNCVLKLKVDTANPKTPKYKFVRIQPGQTPSVLACYNGSPSGVYQCAMRPNPTIKGSFLNGSCGSVGFNIDYDCVSFCYMHHMELFTGVHAGTDLBGNFYGPPVDRQTAQAAGTDTTITVNWVLYAAVINGDRWFLNRPTTTLNDFNLVAMKYNYEPLTQDHVDILGPLSAQTGIAVLDMCASLKELLQNGHNGRTILGSALLEDEFTPFDVVRQCSGVTFQ

SEQ ID NO: 14 - protein, 303 aa, Artificial sequence
  synthetic
SGPRKMAFPSGKVEGCMVQVTCGTTTLNGLWLDDVVYCPRHVICTSEDMLNPNYEDLLIRKSNHNPLVQAGNVQLRVIGHSMQNCVLKLKVDTANPKTPKYKFVRIQPGQTPSVLACYNGSPSGVYQCAMRPNPTIKGSFLNGSCGSVGFNIDYDCVSFCYHHHMELPTGVHAGTDLBGNFYGPFVDRQTAQAAGTDTTITVNWLYAAVINGDRWFLNRPTTTLNDFNLVAMKYNYEPLTQDHVDILGPLSAQTGIAVLDMCASLKELLQNGHNGRTILGSALLEDEFTPFDVVRQCSGVTFQ

SEQ ID NO: 15 - protein, 303 aa, Artificial sequence
  synthetic
SGPRKMAFPSGKVEGCMVQVTCGTTTLNGLWLDDVVYCPRHVICTSEDMLNPNYEDLLIRKSNHNPLVQAGNVQLRVIGHSMQNCVLKLKVDTANPKTPKYKFVRIQPGQTPSVLACYNGSPSGVYQCAMRHNFTIKGSFLNGSCGSVGFNIDYDCVSFCYMHHMELPTGVHAGTDLEGNFYGPFVDRQTAQAAGTDTTITVNWLYAAVINGDRWFLNRPTTTLNDFNLVAMKYNYEPLTQDHVDILGPLSAQTGIAVLDMCASLKELLQNGHNGRTILGSALLEDEFTPFDVVRQCSGVTFQ

(19) United States
(12) Patent Application Publication
(10) Pub. No.: US 2024/0261307 A1
(43) Pub. Date: Aug. 8, 2024

(54) INHIBITORS OF SARS-COV-2 VIRAL REPLICATION AND USES THEREOF

(71) Applicant: ARIZONA BOARD OF REGENTS ON BEHALF OF THE UNIVERSITY OF ARIZONA, Tucson, AZ (US)

(72) Inventors: Rui CHANG, Tucson, AZ (US); Patrick RONALDSON, Tucson, AZ (US); Dominik SCHENTEN, Tucson, AZ (US); Yanyun LIU, Tucson, AZ (US); Ramachandran VIJAYAN, Tucson, AZ (US)

(21) Appl. No.: 18/564,321

(22) PCT Filed: May 27, 2022
(86) PCT No.: PCT/US2022/031360

§ 371 Date: Nov. 27, 2023

Related U.S. Application Data
(60) Provisional application No. 63/194,473, filed on May 28, 2021.

(51) Int. Cl. A61K 31/675; A61K 31/165; A61K 31/195; A61K 31/352; A61K 31/40; A61K 31/437; A61K 31/4706; A61K 31/519; A61K 31/573; A61P 31/14; A61P 37/04

(52) U.S. Cl. CPC A61K 31/675; A61K 31/165; A61K 31/195; A61K 31/352; A61K 31/40; A61K 31/437; A61K 31/4706; A61K 31/519; A61K 31/573; A61P 31/14; A61P 37/04

(57) ABSTRACT

This invention is in the field of medicinal pharmacology. In particular, the present invention relates to pharmaceutical agents which function as inhibitors of severe acute respiratory syndrome coronavirus 2 (SARS-CoV-2) viral replication and/or SARS-CoV-2 related viral 3CL protease (M^{pro}) activity, which function as therapeutics for the treatment of conditions caused by the SARS-CoV-2 virus (e.g., COVID-19), and which function as therapeutics for the treatment conditions related to SARS-CoV-2 related M^{pro} activity.

### CROSS-REFERENCE TO RELATED APPLICATION

The present application claims priority to U.S. Provisional Patent Application No. 63/194,473, filed May 28, 2021, which is hereby incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 40,000 Byte ASCII (Text) file named “39507-601_ST25” created on May 26, 2022.

### FIELD OF THE INVENTION

This invention is in the field of medicinal pharmacology. In particular, the present invention relates to pharmaceutical agents which function as inhibitors of severe acute respiratory syndrome coronavirus 2 (SARS-CoV-2) viral replication and/or SARS-CoV-2 related viral 3CL protease (M^{pro}) activity, which function as therapeutics for the treatment of conditions caused by the SARS-CoV-2 virus (e.g., COVID-19), and which function as therapeutics for the treatment conditions related to SARS-CoV-2 related M^{pro} activity.

### INTRODUCTION

Coronaviruses comprise a large family of positive single stranded RNA viruses that cause respiratory, gastrointestinal, and neurological diseases in humans and other animals 1.2. Severe acute respiratory syndrome coronavirus 2 (SARS-CoV-2) 3-6, the etiological agent of COVID-19 and its ever-increasing evolutionary variants, has become a global health emergency with an urgent need for novel therapeutic strategies to combat the disease. Despite the remarkable and rapid success of vaccines against SARS-CoV-27 in the U.S. and other developed countries, significant infection risk remains among unvaccinated people, immunocompromised or otherwise vulnerable individuals forming a substantial reservoir to support viral spread, which make small-molecule inhibitors of SARS-CoV-2 replication urgently needed. In addition, vaccines mitigate but do not eliminate the likelihood of severe disease. Finally, the emergence of SARS-CoV-2 variants of concern (VOCs), such as the latest strain Omicron, which is highly contagious with increased likelihood to escape vaccine-derived immune surveillance, have raised concerns about the efficacy of the current vaccines, thus illustrating the importance of a wide arsenal of tools to combat the evolving current SARS-CoV-2 strains or novel future coronaviruses altogether.

The SARS-CoV-2 genome encodes several structural proteins including the membrane (M), envelope (E), and spike (S) proteins as well as multiple non-structural proteins that are necessary for viral replication or the manipulation of the host immune response⁸⁻¹⁰. The main protease (Mpro, also known as 3CLPro) is a cysteine protease that is critical for the cleavage of two polypeptide chains encoded by the overlapping open reading frames ORF1a and ORF1b into functional proteins^{11,12}. Among these proteins is the essential RNA polymerase RdRp that is responsible for the replication of viral genome and whose activity is severely compromised without prior proteolytic cleavage by Mpro. In addition to the processing of viral proteins necessary for the viral replication machinery, Mpro has also been suggested to interfere with the induction of cellular type I and type III interferon (IFN) and proinflammatory cytokine responses, either directly through the proteolytic cleavage of members of the IFN signaling cascade or indirectly by promoting the processing of other viral proteins that themselves interfere with IFN signaling¹³⁻¹⁵. The pharmacological inhibition of Mpro may therefore also limit viral replication by inducing a type I and type III IFN-dependent anti-viral state of the host cells.

Improved strategies for inhibiting Mpro activity and for treating SARS-CoV-2 are desperately needed.

The present invention addresses this need.

### SUMMARY

Experiments conducted during the course of developing embodiments for the present invention developed an in-silico pipeline to screen compounds in the ZINC database against Mpro and prioritized 9 lead compounds. Experiments were additional conducted to validate the function of these lead compounds by using replication assays with SARS-CoV-2 in both rhesus monkey kidney-derived Vero cells and human lung-derived Calu-3 cells. Such experiments resulted in the identification of 4 novel compounds that can significantly suppress the replication of SARS-CoV-2 by interfering with Mpro and stimulating the post-infection anti-viral innate immune response.

Accordingly, the present invention relates to pharmaceutical agents which function as inhibitors of severe acute respiratory syndrome coronavirus 2 (SARS-CoV-2) viral replication and/or SARS-CoV-2 related viral 3CL protease (M^{pro}) activity, which function as therapeutics for the treatment of conditions caused by the SARS-CoV-2 virus (e.g., COVID-19), and which function as therapeutics for the treatment conditions related to SARS-CoV-2 related M^{pro} activity.

The compositions, methods, and kits of the present invention are not limited to a particular type or kind of pharmaceutical agent capable of inhibiting SARS-CoV-2 viral replication and/or SARS-CoV-2 related Mpro activity. In some embodiments, the pharmaceutical agent capable of inhibiting SARS-CoV-2 viral replication and/or SARS-CoV-2 related Mpro activity is a small molecule, an antibody, nucleic acid molecule (e.g., siRNA, antisense oligonucleotide), or a mimetic peptide.

Certain small molecule compounds capable of inhibiting SARS-CoV-2 viral replication and/or SARS-CoV-2 related Mpro activity may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

The pharmaceutical agents capable of inhibiting SARS-CoV-2 viral replication and/or SARS-CoV-2 related Mpro activity are configured for any manner of administration (e.g., oral, intravenous, topical).

In a particular embodiment, the pharmaceutical agent capable of inhibiting SARS-CoV-2 viral replication and/or SARS-CoV-2 related Mpro activity is selected from one of the following compounds (or structurally similar compounds):

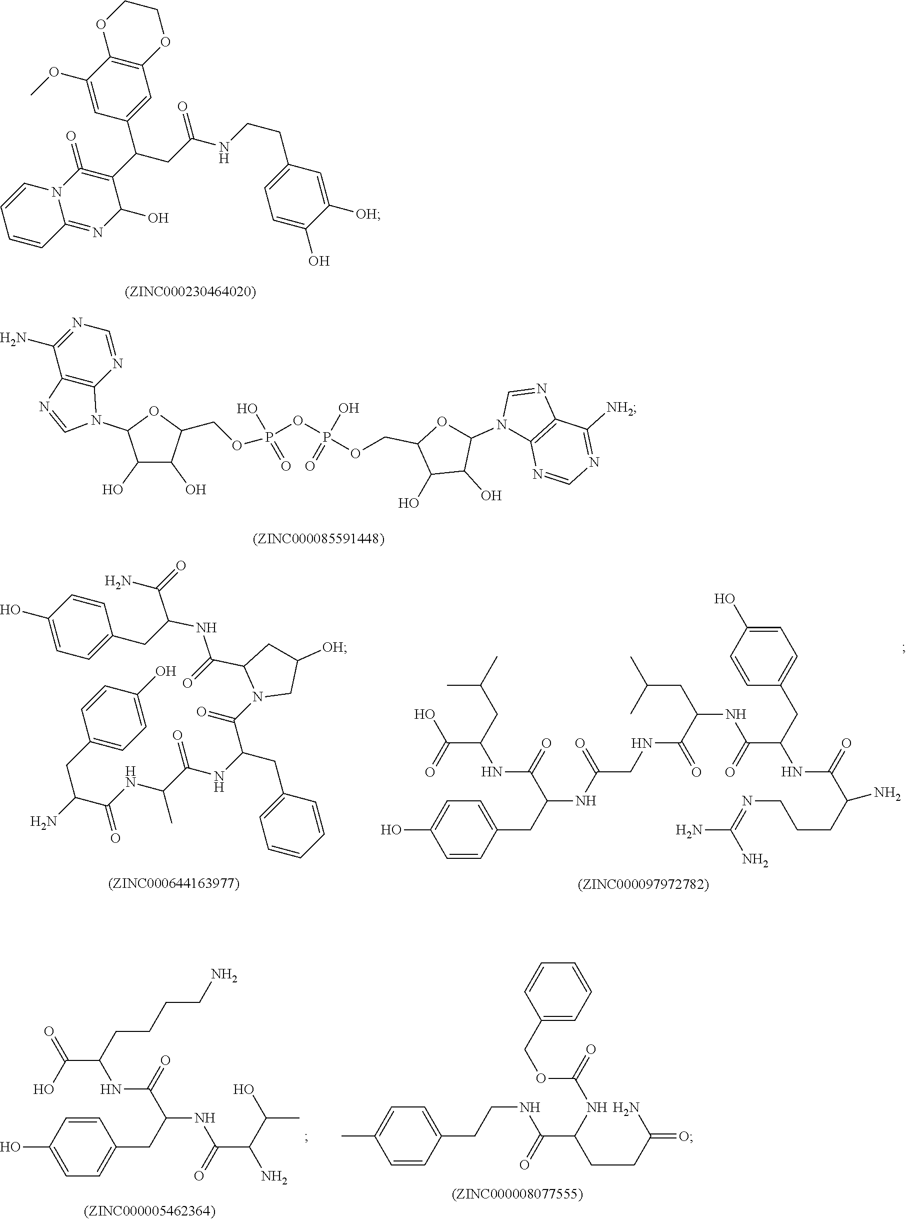

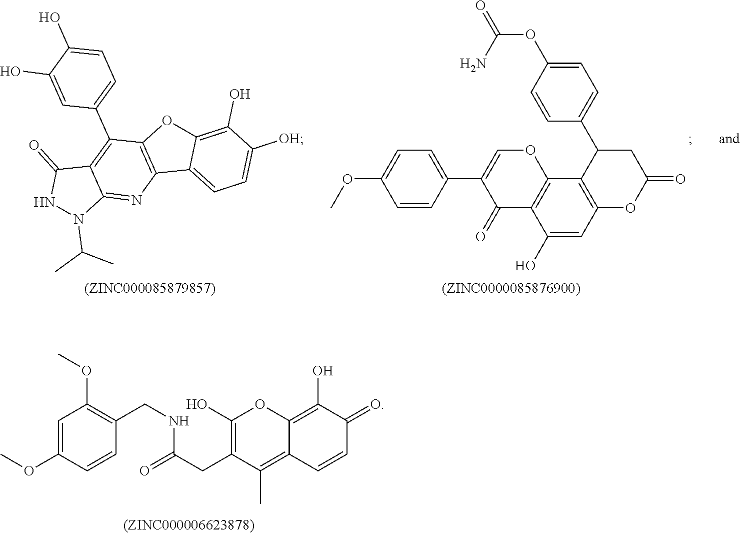 [SMILES: CC(C)n1[nH]c(=O)c2c(-c3ccc(O)c(O)c3)c3oc4c(O)c(O)ccc4c3nc21 ; COc1ccc(-c2coc3c4c(cc(O)c3c2=O)OC(=O)CC4c2ccc(OC(N)=O)cc2)cc1 ; COc1ccc(CNC(=O)Cc2c(O)oc3c(O)c(=O)ccc-3c2C)c(OC)c1]

In a particular embodiment, the pharmaceutical agent capable of inhibiting SARS-CoV-2 viral replication and/or SARS-CoV-2 related Mpro activity is selected from one of the compounds recited in Table 14.

In a particular embodiment, the pharmaceutical agent capable of inhibiting SARS-CoV-2 viral replication and/or SARS-CoV-2 related Mpro activity is capable of engaging (e.g., binding, docking, etc.) within a SARS-CoV-2 M^{pro} binding pocket characterized by one or more of the following SARS-CoV-2 M^{pro} amino acid residues:
THR24, THR25, THR26, LEU27, HIE41, VAL42, CYS44, THR45, SER46, MET49, PRO52, TYR54, ASN119, PHE140, LEU141, ASN142, GLY143, SER144, CYS145, HIE163, HIE164, MET165, GLU166, LEU167, PRO168, GLY170, HIE172, PHE181, ASP187, ARG188, GLN189, THR190, ALA191, and GLN192.

In a particular embodiment, the pharmaceutical agent capable of inhibiting SARS-CoV-2 viral replication and/or SARS-CoV-2 related Mpro activity is capable of engaging (e.g., binding, docking, etc.) within a SARS-CoV-2 M^{pro} binding pocket as shown in FIG. 3A, FIG. 3B, and/or FIG. 6.

The invention further provides processes for preparing any of the pharmaceutical agents capable of inhibiting SARS-CoV-2 viral replication and/or SARS-CoV-2 related Mpro activity as described herein.

In certain embodiments, the present invention provides methods for administering a pharmaceutical composition capable of inhibiting SARS-CoV-2 viral replication and/or SARS-CoV-2 related Mpro activity to a subject (e.g., a human subject) (e.g., a human subject suffering from or at risk of suffering from a condition related to SARS-CoV-2 infection (e.g., COVID-19)) for purposes of treating, preventing and/or ameliorating the symptoms of a viral infection (e.g., SARS-CoV-2 infection (e.g., COVID-19)).

In such embodiments, the methods are not limited treating, preventing and/or ameliorating the symptoms of a particular type or kind of viral infection. In some embodiments, the viral infection is a SARS-CoV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virus having M^{pro} protease activity and/or expression.

In such embodiments, administration of the pharmaceutical composition results in suppression of M^{pro} protease activity within the subject. In some embodiments, administration of the pharmaceutical composition results in production of one or more of type I interferons (IFNs), IFN-sensitive-genes (ISGs), and proinflammatory cytokines. In some embodiments, administration of the pharmaceutical composition results in suppression of any pathway related activity related to M^{pro} protease activity within the subject.

In some embodiments, the pharmaceutical composition capable of inhibiting SARS-CoV-2 viral replication and/or SARS-CoV-2 related Mpro activity is co-administered with one or more of hydroxychloroquine, dexamethasone, and remdesivir.

In certain embodiments, the present invention provides methods for treating, ameliorating and/or preventing a condition related to viral infection in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the viral infection is a SARS-CoV-2 viral infection.

In certain embodiments, the present invention provides methods for treating, ameliorating and/or preventing SARS-CoV-2 infection (e.g., COVID-19) in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject.

In certain embodiments, the present invention provides methods for treating, ameliorating and/or preventing symptoms related to viral infection in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing symptoms related to SARS-CoV-2 infection (e.g., COVID-19) in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing acute respiratory distress syndrome related to SARS-CoV-2 infection (e.g., COVID-19) in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia related to SARS-CoV-2 infection (e.g., COVID-19) in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments involving the treatment of acute respiratory distress syndrome and/or pneumonia, the pharmaceutical composition is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include HI antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisturization by nebulization, anti-tussives, respiratory stimulants, surgery and alpha 1 antitrypsin.

In certain embodiments, the present invention provides methods for inhibiting viral entry in a cell, comprising exposing the cell to a pharmaceutical composition capable of inhibiting SARS-CoV-2 viral replication and/or SARS-CoV-2 related Mpro activity. In some embodiments, the cell is at risk of viral infection (e.g., a cell at risk of SARS-CoV-2 infection). In some embodiments, the cell has been exposed to a virus (e.g., a cell currently exposed to SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, exposure of the cell to the pharmaceutical composition results in suppression of M^{pro} activity within the cell.

In certain embodiments, the present invention provides methods for inhibiting viral replication in a cell, comprising exposing the cell a composition capable of inhibiting SARS-CoV-2 viral replication and/or SARS-CoV-2 related Mpro activity. In some embodiments, the cell is a virus infected cell (e.g., a cell infected with SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, the viral replication is SARS-CoV-2 viral replication. In some embodiments, the viral replication is reducted by about 50%. In some embodiments, the viral replication is reducted by about 25%. In some embodiments, the viral replication is reducted by about 75%. In some embodiments, the viral replication is reducted by about 99.999%.

In certain embodiments, the present invention provides kits comprising a pharmaceutical composition capable of inhibiting SARS-CoV-2 viral replication and/or SARS-CoV-2 related Mpro activity, and one or more of (1) a container, pack, or dispenser, (2) one or more additional agents selected from hydroxychloroquine, dexamethasone, and remdesivir, and (3) instructions for administration.

Such methods are not limited to a particular type or kind of viral infection. In some embodiments, the viral infection is a SARS-CoV-2 related viral infection. In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virals having M^{pro} protease activity and/or expression.

### BRIEF DESCRIPTION OF THE DRAWINGS

FIG. 1A-B: The structure of Mpro and Spike protein across SARS-CoV-2 variants. (A) The active site residues of Mpro are highlighted in red color. Omicron-specific mutation. P132H (B. 1.1529) of Mpro protein are highlighted in blue. Mutations of Mpro protein (K90R, L205V) from the B.1.351 and P.2 variants are highlighted in black. (B) The active site residues of Spike protein are highlighted in red. Omicron-specific mutations of Spike protein are highlighted in blue. Mutations of Spike protein from all the other VOCs are highlighted in black.

FIG. 2: Sequence comparison of Mpro across SARS-CoV-2 variants and other human b-coronaviruses. The locations of non-synonymous mutations resulting in a lysine to arginine mutation in the SARS-CoV-2 B.1.351 (Beta) variant, a leucine to valine mutation in the SARS-CoV-2 P.2 (Zeta) variant, and proline to histidine mutation in the SARS-CoV-2 B.1.1.529 (Omicron) variant are shown in red box.

FIG. 3A: Sequence comparison of Spike protein across SARS-CoV-2 variants and other human b-coronaviruses. Mutations of Spike protein across all VOCs of SARS-CoV-2 are highlighted in red box. The mutation rate was significantly higher in Spike protein with 78 mutations (substitution, deletion and insertion).

FIG. 3B: Inter-molecular interactions of ZINC000085591448, ZINC000097972782, ZINC000005462364, ZINC000644163977, ZINC000008077555, ZINC000230464020, ZINC000006623878, ZINC000085876900 and ZINC000085879857 and SARS-CoV-2 M^{pro}.

FIG. 4: In-silico screening workflow of SARS-CoV-2 Mpro inhibitor. Workflow for the identification of potential small molecular inhibitors for SARS-CoV-2 Mpro. The structure-based virtual screening was carried out using Schrödinger. MOE. Vina. After virtual screening, the top-ranked 500 lead compounds were prioritized based on the Schrödinger Glide score and further independently prioritized using two strategies that ultimately led to total of 9 lead molecules for in-vitro testing.

FIG. 5A-E: Suppression of SARS-CoV-2 infection by putative Mpro inhibitors in Vero cells. Vero cells were infected with SARS-CoV-2 in the presence of increasing concentrations of (A) ZINC000644163977, (B) ZINC00000807555, (C) ZINC0002304644020, (D) ZINC000006623878, or (E) ZINC000085876900. The reduction of treated cells relative to untreated controls was measured 4 days later by plaque assay. Shown are the combined data of at least three independent experiments.

FIG. 6: Modeling of molecular interactions of lead Mpro inhibitors with the active site of SARS-CoV-2 Mpro protein. Shown are the interactions for (A) ZINC000230464020, (B) ZINC000085591448 (C), ZINC000644163977 (D) ZINC000097972782 (E) ZINC000005462364 (F) ZINC000008077555 (G) ZINC000085879857 (H) ZINC000085876900 and (I) ZINC000006623878. All the compounds are shown in green color. Binding pocket of Mpro are shown in brown. The interacting residues at the Mpro active site residues are highlighted in brown sticks.

FIG. 7A-E: CPE and cytotoxicity in Vero cells treated with putative SARS-CoV-2 Mpro inhibitors. (A-E) Vero cells were incubated with the P-gp inhibitor CP-100356 and infected with SARS-CoV-2 in the presence of increasing concentrations of putative Mpro inhibitors. CPE (red line) and cytotoxicity (black line) of the inhibitors was determined 3 days later and expressed as fraction of adherent cells in infected samples relative to uninfected controls (CPE) or untreated cells (cytotoxicity). The amount of adherent cells was quantified by the spectrophotometrical measurement of crystal violet staining. Shown are the results of (A) ZINC000644163977, (B) ZINC00000807555, (C) ZINC0002304644020, (D) ZINC000006623878, and (E) ZINC000085876900. Each experiment was set-up in triplicates and independently repeated twice. The EC50 of viral inhibition and the 95% confidence interval is indicated in each graph.

FIG. 8A-E: Suppression of SARS-CoV-2 infection by putative Mpro inhibitors in Calu-3 cells. Calu-3 cells were infected with SARS-CoV-2 in the presence of increasing concentrations of (A) ZINC000644163977, (B) ZINC00000807555, (C) ZINC0002304644020, (D) ZINC000006623878, and (E) ZINC000085876900. The reduction of the viral titers of treated samples relative to untreated controls was measured two days later by quantifying the viral titers in serial dilutions of the Calu-3 supernatants using a Vero cell-based plaque assay. Shown are the combined data of at least three independent experiments.

FIG. 9A-H: Mpro inhibitors ZINC00000807555, ZINC0002304644020, ZINC000006623878, and ZINC000085876900 suppress SARS-CoV-2 replication and do not impact WNV replication. Replication of SARS-CoV-2 (A-D) and WNV (E-H) in the presence of 100 μM of (A, E) ZINC00000807555, (B, F) ZINC0002304644020, (C, G) ZINC000006623878, or (D, H) ZINC000085876900. Viral titers in the culture medium of SARS-CoV-2 or WNV-infected Vero cells were measured over the course of two days post infection by viral plaque assay (SARS-CoV-2) or viral foci assay (WNV). Shown are the combined data of at least 3 independent experiments.

FIG. 10A-B: Pre-treatment and post-treatment of SARS-CoV-2-infected cells with Mpro. (A) Pre-treatment. Vero cells were treated with 100 mM of ZINC0002304644020, ZINC000006623878, ZINC000085876900, or all compounds 24 hrs prior to infection with SARS-CoV-2 24 hrs. Vehicle-treated cells served as controls. (B) Post-treatment. Vero cells were infected with SARS-CoV-2 and treated 24 hrs later with 100 mM ZINC0002304644020, ZINC000006623878, ZINC000085876900, or all compounds. Viral titers in the cell supernatants were measured two days after treatment. Vehicle-treated cells served as controls. (A-B) Viral titers were measured by plaque assay using serial dilutions of the cell supernatants. Shown are the viral titers relative to the vehicle-treated control samples. Data represent the combined results from at least three experiments. *, p≤0.05; **, p≤0.005; ***, p≤0.0005, Mann-Whitney test.

FIG. 11A-D: ZINC0002304644020 and ZINC000085876900 promote enhanced type I IFN, interferon-sensitive genes (ISGs), and cytokine responses of SARS-CoV-2-infected Calu-3 cells despite reduced viral RNA expression. (A-C) Expression of (A) type I IFNs. (B) ISGs. and (C) proinflammatory cytokines in SARS-CoV-2-infected Calu-3 cells as measured by RT-qPCR 18 hrs post infection. Shown is the gene expression in cells treated with 100 mM of Mpro inhibitors relative to that of uninfected naïve cells. Uninfected cells treated with Mpro inhibitors were included as controls. (D) Expression of viral RNA by ZINC0002304644020 and ZINC000085876900 as measured by RT-qPCR. (A-D) Data represent the combined results of three independent experiments. CoV-2, SARS-CoV-2; ZINC-020, ZINC0002304644020; ZINC-900, ZINC000085876900; *, p≤0.05; **, p≤0.005; ***, p≤0.0005. Anova test with multiple comparisons.

FIG. 12A-D: ZINC0002304644020 and ZINC000085876900 promote enhanced type I IFN, interferon-sensitive genes (ISGs), and cytokine responses of SARS-CoV-2-infected Vero cells despite reduced viral RNA expression. (A-C) Expression of (A) type I IFNs. (B) ISGs. and (C) proinflammatory cytokines in SARS-CoV-2-infected Calu-3 cells as measured by RT-qPCR 18 hrs post infection. Shown is the gene expression in cells treated with 100 mM of Mpro inhibitors relative to that of uninfected naïve cells. Uninfected cells treated with Mpro inhibitors were included as controls. (D) Expression of viral RNA by ZINC0002304644020 and ZINC000085876900 as measured by RT-qPCR. (A-D) Data represent the combined results of three independent experiments. CoV-2, SARS-CoV-2; ZINC-020, ZINC0002304644020; ZINC-900, ZINC000085876900; *, p≤0.05; **, p≤0.005; ***, p≤0.0005. Anova test with multiple comparisons.

FIG. 13A-B: ZINC0002304644020 and ZINC000085876900 promote enhanced type I IFN, interferon-sensitive genes (ISGs), and cytokine responses of SARS-CoV-2-infected cells especially when normalized to level of viral RNA expression. (A-B) Expression of type I IFNs. ISGs. and (C) proinflammatory cytokines in SARS-CoV-2-infected (A) Calu-3 cells and (B) Vero cells as measured by RT-qPCR 18 hrs post infection. Shown is the gene expression in cells treated with 100 mM of Mpro inhibitors relative to that of infected but untreated cells. The gene expression levels of each sample were normalized to the level of viral RNA found in the same sample. Data represent the combined results of three independent experiments. CoV-2, SARS-CoV-2; ZINC-020, ZINC0002304644020; ZINC-900, ZINC000085876900; *, p≤0.05; **, p≤0.005; ***, p≤0.0005. Anova test with multiple comparisons.

FIG. 14: The top 500 lead compounds obtained from virtual screening ranked according to their Glide score (see. Example I).

FIG. 15: Prioritization of lead inhibitors of SARS-CoV-2 Mpro protein based on pharmacology-informed approach.

FIG. 16: Activity of SARS-CoV-2 Mpro Inhibitors in vitro Vero cells were infected with SARSCOV-2 (strain WA1, MOI=0.005) in the presence of 10-100 μM of the indicated compounds and 2 μM of the P-gp inhibitor CP-100356. Vehicle-treated cells served as controls. The amounts of infectious virions in the cell supernatants were determined 2 days later by viral plaque assay. ZINC000085876900 was toxic at 100 μM. *, p≤0.05; **, p≤0.005, ***, p≤0.0005; Mann-Whitney test.

### DETAILED DESCRIPTION OF THE INVENTION

Severe acute respiratory syndrome coronavirus 2 (SARS-CoV-2), the etiologic agent of COVID-19, can cause severe disease with high mortality rates, especially among older and vulnerable populations. Despite the recent success of vaccines and approval of first-generation anti-viral inhibitor against SARS-CoV-2, an expanded arsenal of anti-viral compounds that limit viral replication and ameliorate disease severity is still urgently needed in light of the continued emergence of viral variants of concern (VOC). The main protease (Mpro) of SARS-CoV-2 is the major non-structural protein required for the processing of viral polypeptides encoded by the open reading frame 1 (ORF1) and ultimately replication. Structural conservation of Mpro among SARS-CoV-2 variants make this protein an attractive target for the anti-viral inhibition by small molecules.

Experiments conducted during the course of developing embodiments for the present invention resulted in the development of a structure-based in-silico screening of approximately 11 million compounds in ZINC database inhibiting Mpro, which prioritized 9 lead compounds for the subsequent in vitro validation in SARS-CoV-2 replication assays using both Vero and Calu-3 cells. Additional experiments validated that four of these lead compounds significantly inhibited SARS-CoV-2 replication in the micromolar range. Importantly, it was demonstrated that such compounds not only limited viral replication via inhibition of Mpro but also stimulated the production of type I interferons (IFNs), IFN-sensitive-genes (ISGs), and proinflammatory cytokines, thus implicating the anti-viral innate immune response as part of the drug mechanism. As such, such experiments resulted in the identification of novel small-molecules capable of significantly suppressing infection and replication of SARS-CoV-2 and stimulating anti-viral immune response in human cells.

Accordingly, the present invention relates to pharmaceutical agents which function as inhibitors of severe acute respiratory syndrome coronavirus 2 (SARS-CoV-2) viral replication and/or SARS-CoV-2 related viral 3CL protease (M^{pro}) activity, which function as therapeutics for the treatment of conditions caused by the SARS-CoV-2 virus (e.g., COVID-19), and which function as therapeutics for the treatment conditions related to SARS-CoV-2 related M^{pro} activity.

The compositions, methods, and kits of the present invention are not limited to a particular type or kind of pharmaceutical agent capable of inhibiting SARS-CoV-2 viral replication and/or SARS-CoV-2 related Mpro activity. In some embodiments, the pharmaceutical agent capable of inhibiting SARS-CoV-2 viral replication and/or SARS-CoV-2 related Mpro activity is a small molecule, an antibody, nucleic acid molecule (e.g., siRNA, antisense oligonucleotide), or a mimetic peptide.

Certain small molecule compounds capable of inhibiting SARS-CoV-2 viral replication and/or SARS-CoV-2 related Mpro activity may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

The pharmaceutical agents capable of inhibiting SARS-CoV-2 viral replication and/or SARS-CoV-2 related Mpro activity are configured for any manner of administration (e.g., oral, intravenous, topical).

In a particular embodiment, the pharmaceutical agent capable of inhibiting SARS-CoV-2 viral replication and/or SARS-CoV-2 related Mpro activity is selected from one of the following compounds (or structurally similar compounds):

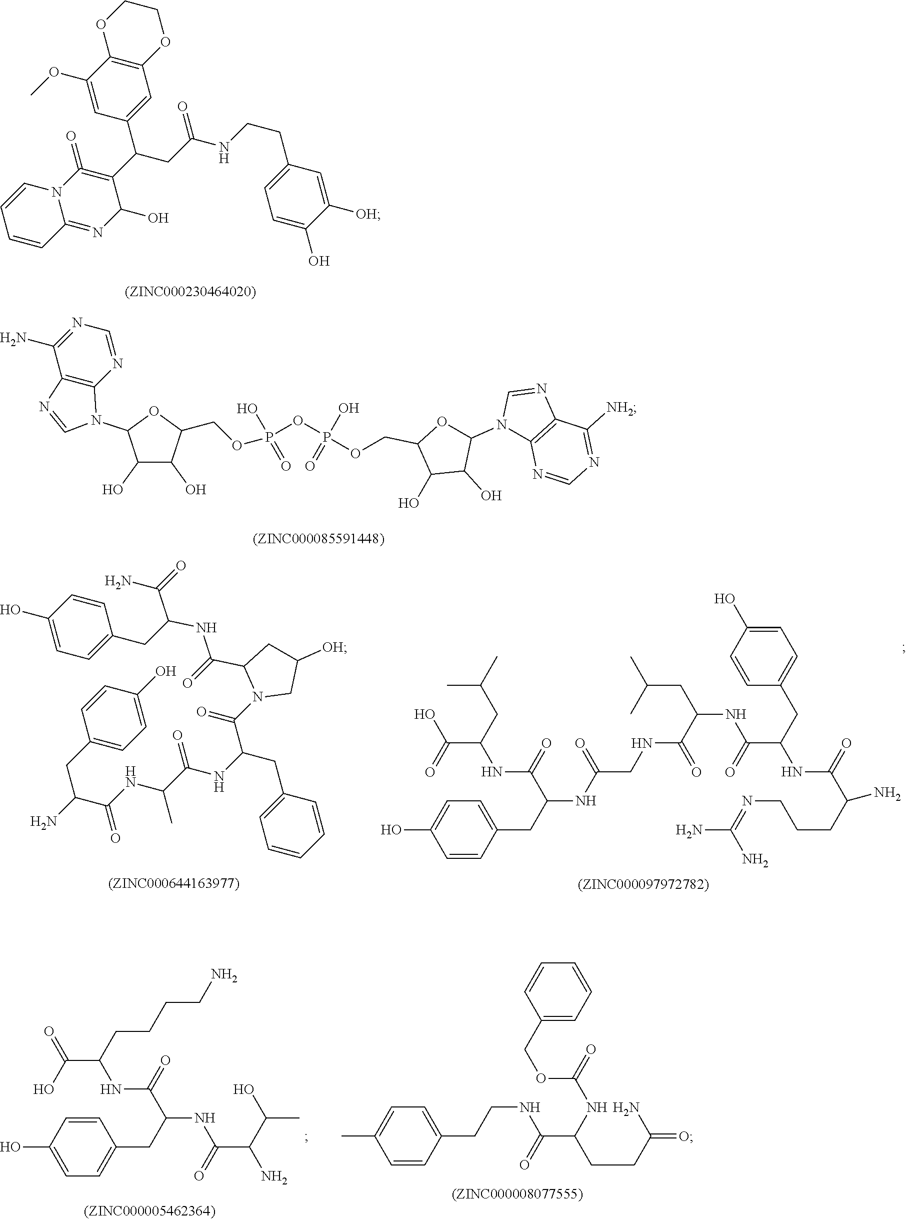

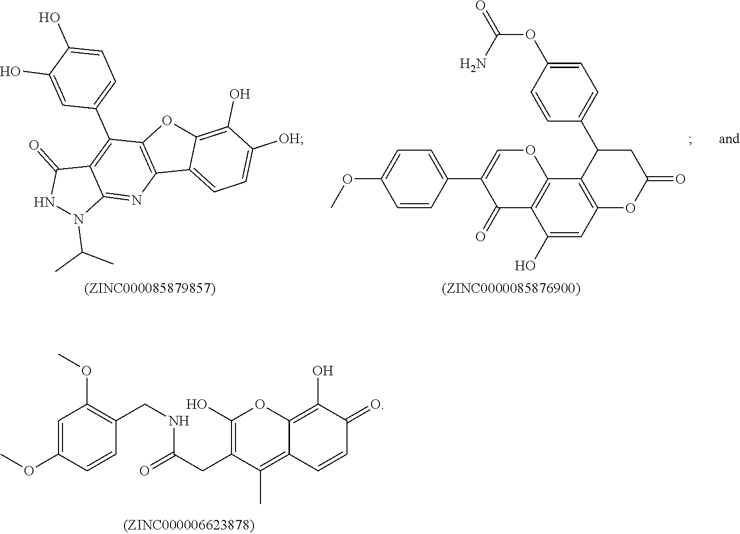 [SMILES: CC(C)n1[nH]c(=O)c2c(-c3ccc(O)c(O)c3)c3oc4c(O)c(O)ccc4c3nc21 ; COc1ccc(-c2coc3c4c(cc(O)c3c2=O)OC(=O)CC4c2ccc(OC(N)=O)cc2)cc1 ; COc1ccc(CNC(=O)Cc2c(O)oc3c(O)c(=O)ccc-3c2C)c(OC)c1]

In a particular embodiment, the pharmaceutical agent capable of inhibiting SARS-CoV-2 viral replication and/or SARS-CoV-2 related Mpro activity is selected from one of the compounds recited in Table 14.

In a particular embodiment, the pharmaceutical agent capable of inhibiting SARS-CoV-2 viral replication and/or SARS-CoV-2 related Mpro activity is capable of engaging (e.g., binding, docking, etc.) within a SARS-CoV-2 M^{pro} binding pocket characterized by one or more of the following SARS-CoV-2 M^{pro} amino acid residues:
THR24, THR25, THR26, LEU27, HIE41, VAL42, CYS44, THR45, SER46, MET49, PRO52, TYR54, ASN119, PHE140, LEU141, ASN142, GLY143, SER144, CYS145, HIE163, HIE164, MET165, GLU166, LEU167, PRO168, GLY170, HIE172, PHE181, ASP187, ARG188, GLN189, THR190, ALA191, and GLN192.

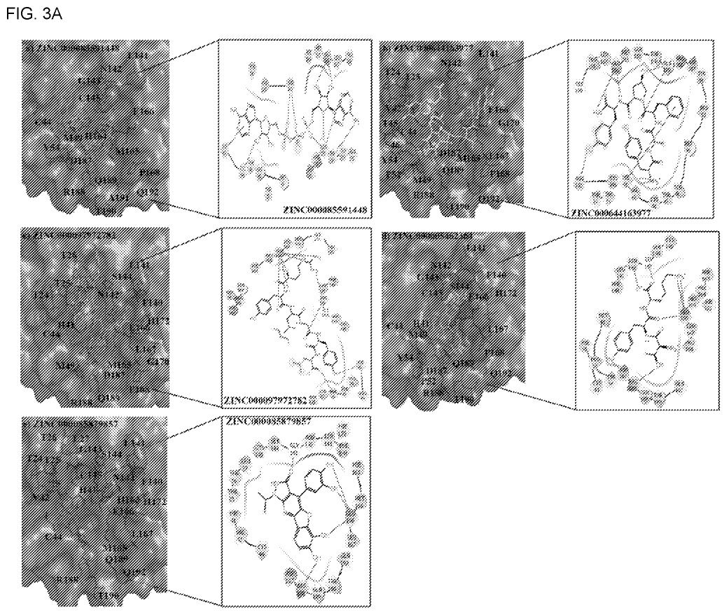

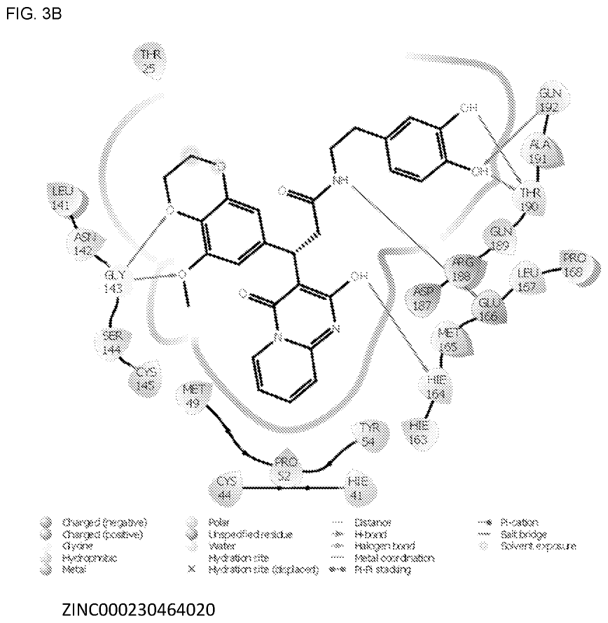

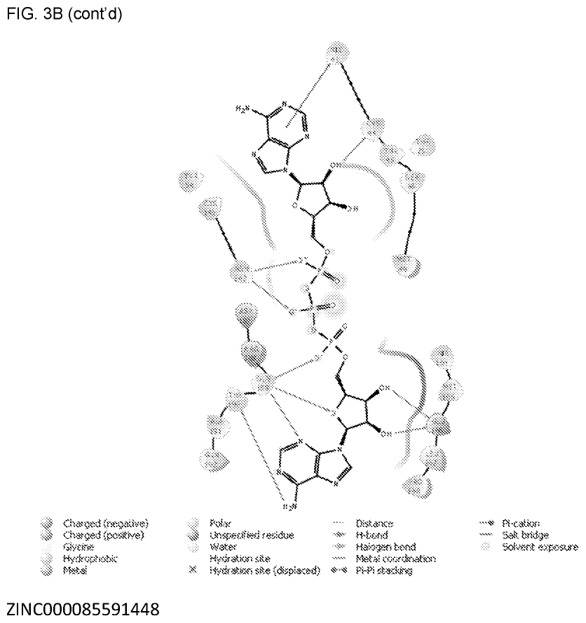

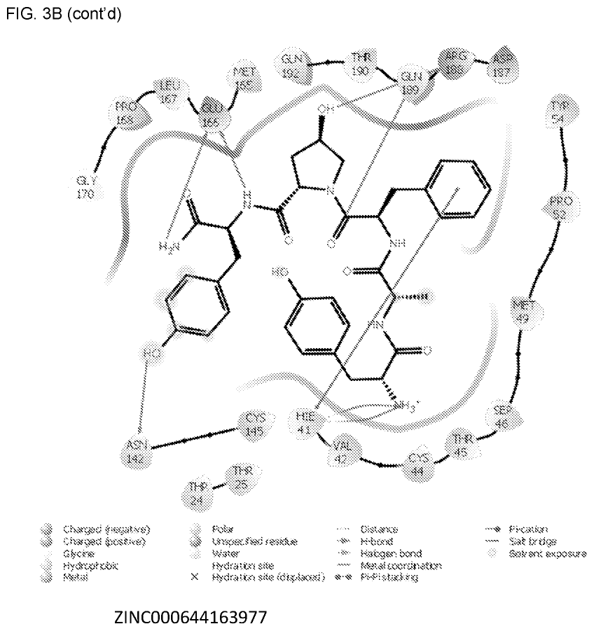

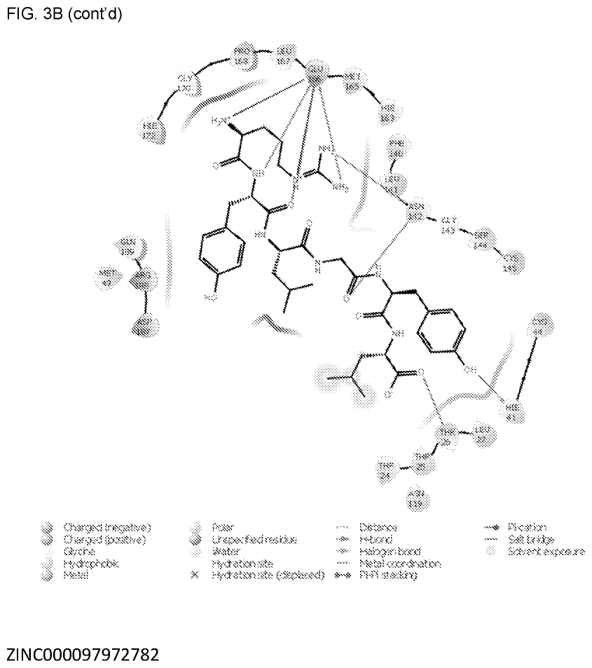

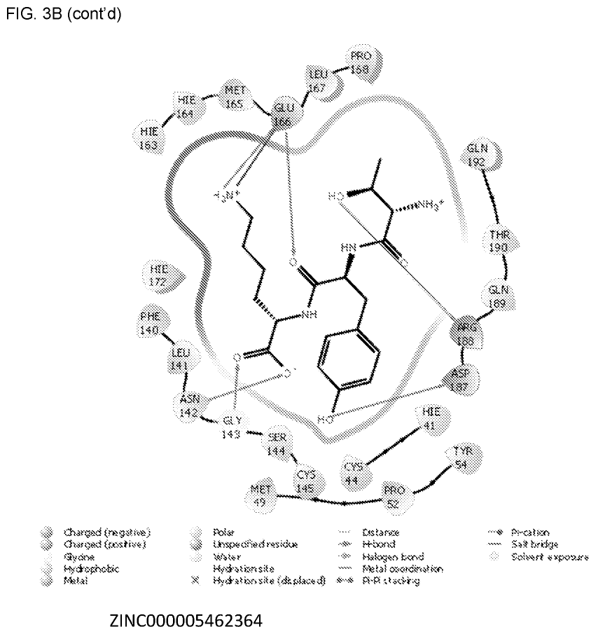

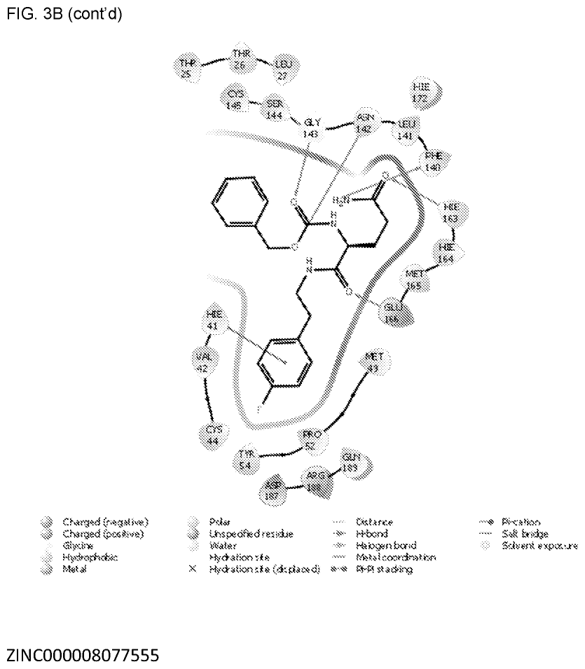

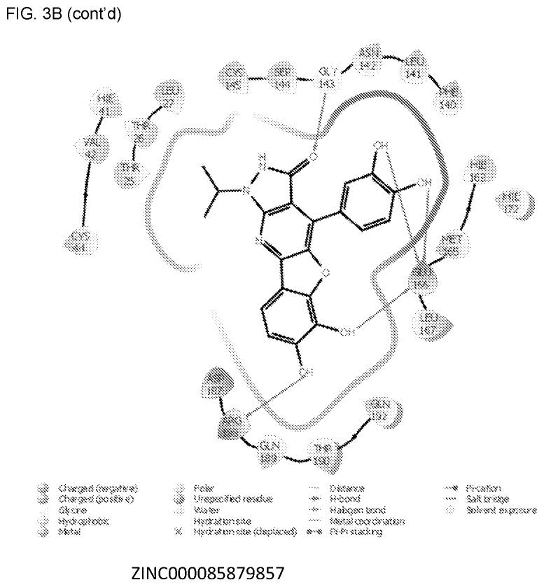

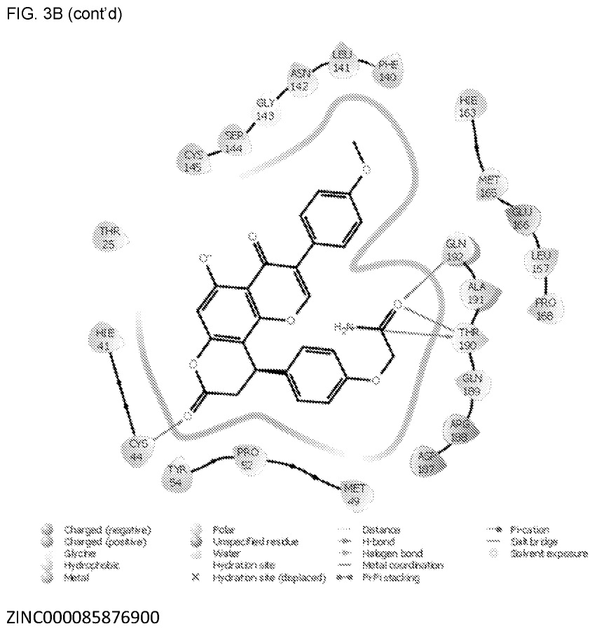

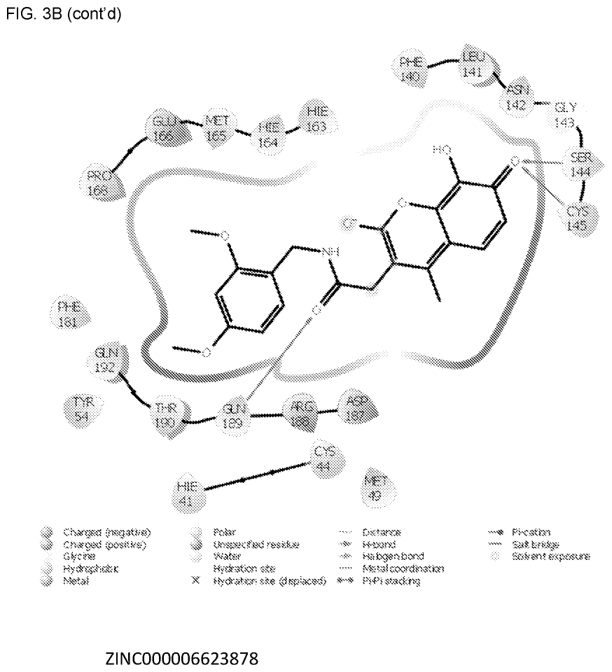

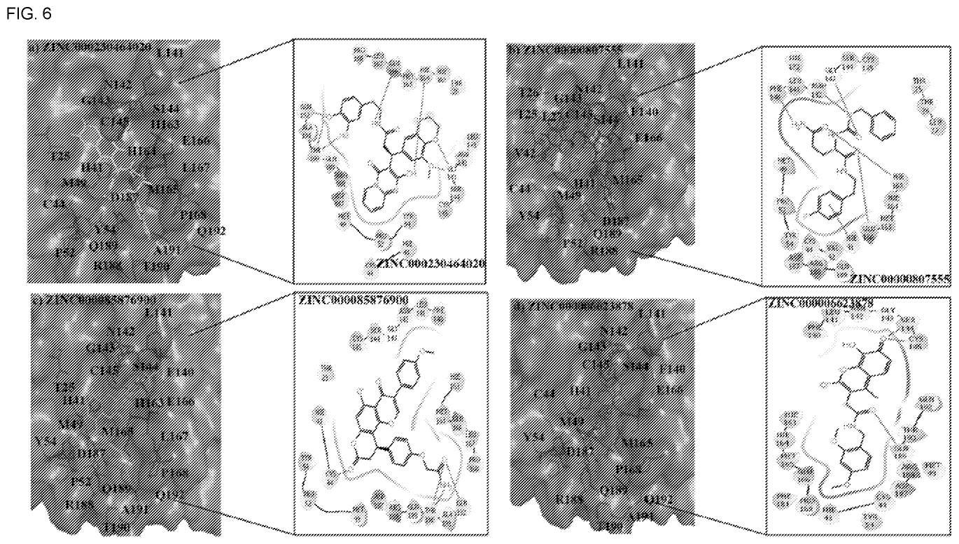

In a particular embodiment, the pharmaceutical agent capable of inhibiting SARS-CoV-2 viral replication and/or SARS-CoV-2 related Mpro activity is capable of engaging (e.g., binding, docking, etc.) within a SARS-CoV-2 M^{pro} binding pocket as shown in FIG. 3A, FIG. 3B, and/or FIG. 6.

The invention further provides processes for preparing any of the pharmaceutical agents capable of inhibiting SARS-CoV-2 viral replication and/or SARS-CoV-2 related Mpro activity as described herein.

In certain embodiments, the present invention provides methods for administering a pharmaceutical composition capable of inhibiting SARS-CoV-2 viral replication and/or SARS-CoV-2 related Mpro activity to a subject (e.g., a human subject) (e.g., a human subject suffering from or at risk of suffering from a condition related to SARS-CoV-2 infection (e.g., COVID-19)) for purposes of treating, preventing and/or ameliorating the symptoms of a viral infection (e.g., SARS-CoV-2 infection (e.g., COVID-19)).

In such embodiments, the methods are not limited treating, preventing and/or ameliorating the symptoms of a particular type or kind of viral infection. In some embodiments, the viral infection is a SARS-CoV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV. SARS coronavirus, Tacaribe virus, and West Nile virus. In some embodiments, the viral infection is associated with any virus having M^{pro} protease activity and/or expression.

In such embodiments, administration of the pharmaceutical composition results in suppression of M^{pro} protease activity within the subject. In some embodiments, administration of the pharmaceutical composition results in production of one or more of type I interferons (IFNs), IFN-sensitive-genes (ISGs), and proinflammatory cytokines. In some embodiments, administration of the pharmaceutical composition results in suppression of any pathway related activity related to M^{pro} protease activity within the subject.

In some embodiments, the pharmaceutical composition capable of inhibiting SARS-CoV-2 viral replication and/or SARS-CoV-2 related Mpro activity is co-administered with one or more of hydroxychloroquine, dexamethasone, and remdesivir.

In certain embodiments, the present invention provides methods for treating, ameliorating and/or preventing a condition related to viral infection in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the viral infection is a SARS-CoV-2 viral infection.

In certain embodiments, the present invention provides methods for treating, ameliorating and/or preventing SARS-CoV-2 infection (e.g., COVID-19) in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject.

In certain embodiments, the present invention provides methods for treating, ameliorating and/or preventing symptoms related to viral infection in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing symptoms related to SARS-CoV-2 infection (e.g., COVID-19) in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing acute respiratory distress syndrome related to SARS-CoV-2 infection (e.g., COVID-19) in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia related to SARS-CoV-2 infection (e.g., COVID-19) in a subject, comprising administering to the subject a pharmaceutical composition capable of inhibiting SARS-CoV-2 viral replication and/or SARS-CoV-2 related Mpro activity. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments involving the treatment of acute respiratory distress syndrome and/or pneumonia, the pharmaceutical composition is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include HI antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisturization by nebulization, anti-tussives, respiratory stimulants, surgery and alpha 1 antitrypsin.

In certain embodiments, the present invention provides methods for inhibiting viral entry in a cell, comprising exposing the cell to a pharmaceutical composition capable of inhibiting SARS-CoV-2 viral replication and/or SARS-CoV-2 related Mpro activity. In some embodiments, the cell is at risk of viral infection (e.g., a cell at risk of SARS-CoV-2 infection). In some embodiments, the cell has been exposed to a virus (e.g., a cell currently exposed to SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, exposure of the cell to the pharmaceutical composition results in suppression of M^{pro} activity within the cell.

In certain embodiments, the present invention provides methods for inhibiting viral replication in a cell, comprising exposing the cell a composition capable of inhibiting SARS-CoV-2 viral replication and/or SARS-CoV-2 related Mpro activity. In some embodiments, the cell is a virus infected cell (e.g., a cell infected with SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, the viral replication is SARS-CoV-2 viral replication. In some embodiments, the viral replication is reducted by about 50%. In some embodiments, the viral replication is reducted by about 25%. In some embodiments, the viral replication is reducted by about 75%. In some embodiments, the viral replication is reducted by about 99.999%.

In certain embodiments, the present invention provides kits comprising a pharmaceutical composition capable of inhibiting SARS-CoV-2 viral replication and/or SARS-CoV-2 related Mpro activity, and one or more of (1) a container, pack, or dispenser, (2) one or more additional agents selected from hydroxychloroquine, dexamethasone, and remdesivir, and (3) instructions for administration.

Compositions within the scope of this invention include all pharmaceutical compositions contained in an amount that is effective to achieve its intended purpose. While individual needs vary, determination of optimal ranges of effective amounts of each component is within the skill of the art. Typically, the pharmaceutical agents which function as inhibitors of M^{pro} protease activity may be administered to mammals, e.g. humans, orally at a dose of 0.0025 to 50 mg/kg, or an equivalent amount of the pharmaceutically acceptable salt thereof, per day of the body weight of the mammal being treated. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the inhibiting agent. The unit dose may be administered one or more times daily as one or more tablets or capsules each containing from about 0.1 to about 10 mg, conveniently about 0.25 to 50 mg of the agent (e.g., small molecule) or its solvates.

In a topical formulation, a compound of the present invention may be present at a concentration of about 0.01 to 100 mg per gram of carrier. In a one embodiment, such a compound is present at a concentration of about 0.07-1.0 mg/ml, for example, about 0.1-0.5 mg/ml, and in one embodiment, about 0.4 mg/ml.

In addition to administering a compound of the present invention as a raw chemical, it may be administered as part of a pharmaceutical preparation containing suitable pharmaceutically acceptable carriers comprising excipients and auxiliaries which facilitate processing of the compound into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active mimetic peptide(s), together with the excipient.

The pharmaceutical compositions of the invention may be administered to any patient that may experience the beneficial effects of one or more of compounds of the present invention. Foremost among such patients are mammals, e.g., humans, although the invention is not intended to be so limited. Other patients include veterinary animals (cows, sheep, pigs, horses, dogs, cats and the like).

The pharmaceutical compositions comprising a compound of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is itself known, for example, by means of conventional mixing, granulating, dragee-making, dissolving, or lyophilizing processes. Thus, pharmaceutical preparations for oral use can be obtained by combining the active mimetic peptides with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye-stuffs or pigments may be added to the tablets or dragee coatings, for example, for identification or in order to characterize combinations of active mimetic peptide doses.

Other pharmaceutical preparations that can be used orally include push-fit capsules made of gelatin, as well as soft, sealed capsules made of gelatin and a plasticizer such as glycerol or sorbitol. The push-fit capsules can contain the active mimetic peptides in the form of granules that may be mixed with fillers such as lactose, binders such as starches, and/or lubricants such as talc or magnesium stearate and, optionally, stabilizers. In soft capsules, the active mimetic peptides are in one embodiment dissolved or suspended in suitable liquids, such as fatty oils, or liquid paraffin. In addition, stabilizers may be added.

Possible pharmaceutical preparations that can be used rectally include, for example, suppositories, which consist of a combination of one or more of the active mimetic peptides with a suppository base. Suitable suppository bases are, for example, natural or synthetic triglycerides, or paraffin hydrocarbons. In addition, it is also possible to use gelatin rectal capsules that consist of a combination of the active mimetic peptides with a base. Possible base materials include, for example, liquid triglycerides, polyethylene glycols, or paraffin hydrocarbons.

Suitable formulations for parenteral administration include aqueous solutions of the active mimetic peptides in water-soluble form, for example, water-soluble salts and alkaline solutions. In addition, suspensions of the active mimetic peptides as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Ointments may be formulated by mixing a solution of the active ingredient in a vegetable oil such as almond oil with warm soft paraffin and allowing the mixture to cool. A typical example of such an ointment is one that includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### EXPERIMENTAL

As used herein, personal pronouns (e.g., “I”, “we”, “our”, etc.) refer to the inventors who conducted and/or directed the described experiments.

### Example I

### Mpro is Conserved Among SARS-CoV-2 Variants of Concern.

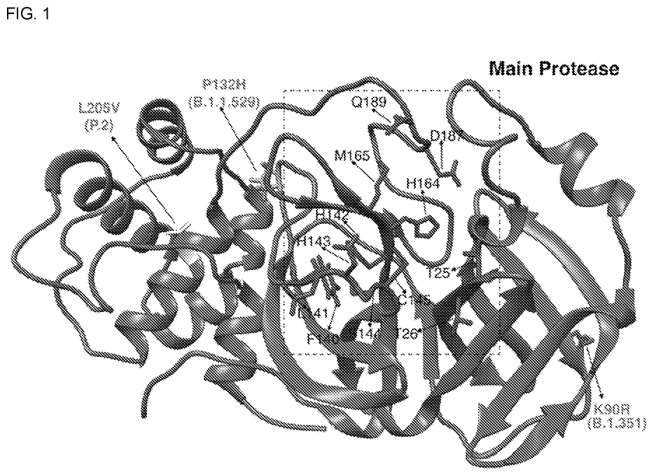

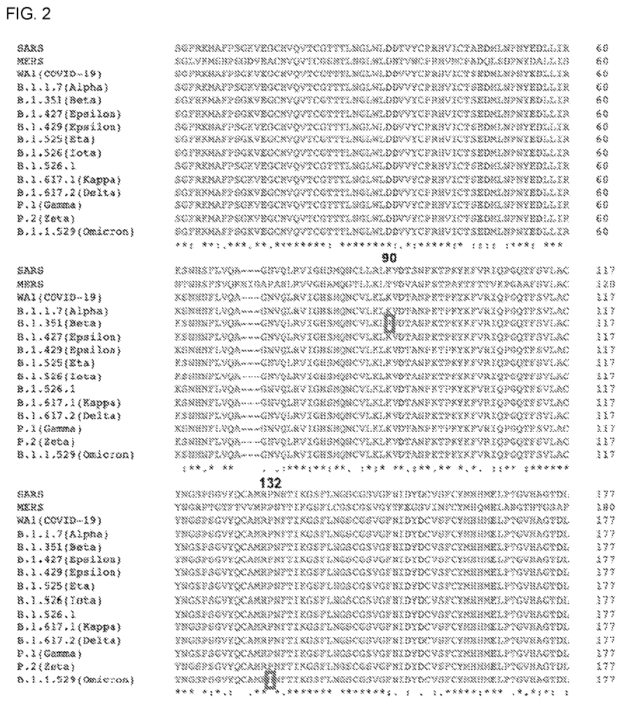

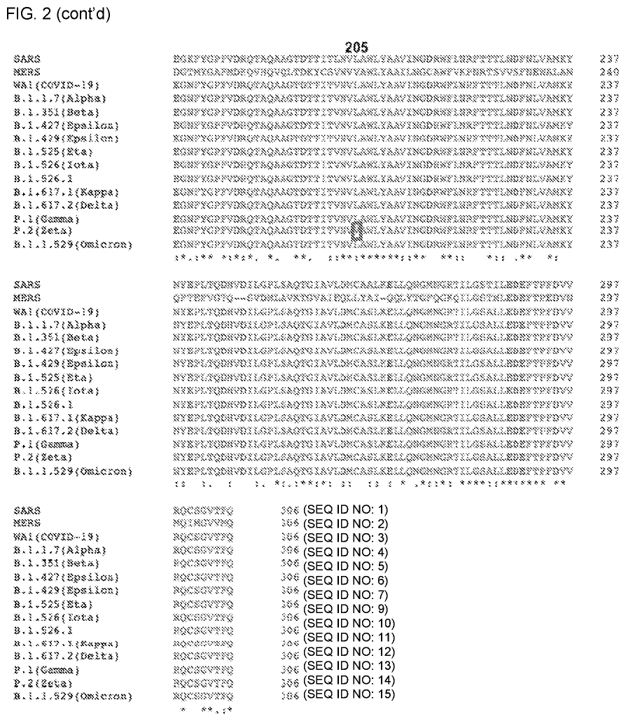

The frequent occurrence of mutations in the viral spike (S) protein among SARS-CoV-2 VOCs suggest that the S protein of SARS-CoV-2 remains under evolutionary pressure to adapt to the human ACE2 receptor. Mpro may be less sensitive to such selective pressure as it has a substrate specificity for viral proteins with unique glutamate-containing cleavage sites that are distinct from the sites used by known human proteases¹⁶. To evaluate whether Mpro is indeed structurally conserved among known SARS-CoV-2 strains, we compared the consensus sequence of original North-American WA1 strain to a range of SARS-CoV-2 variants of concern, including B.1.1.7(Alpha), B.1.351(Beta), B.1.427 & B.1.429 (Epsilon), B.1.525 (Eta), B.1526 (Iota), B.1.526.1, B.1.617.1 (Kappa), B.1.617.2 (Delta), P.1 (Gamma), P.2 (Zeta) and B.1.1.529 (Omicron), as well as the more distantly related human coronaviruses SARS-COV and MERS. We found that there are only three substitutions (K90R, L205V, P132H) in Mpro across all VOCs of SARS-CoV-2 (FIG. 1A, FIG. 2, Table 1). The recently determined X-ray crystal structure of Mpro (PDB ID: 6W63) reveals four distinct regions of the main protease protomer, namely Domain I (residue 10-99) and Domain II (residue 100-184) that form antiparallel β-barrels, followed by a long connecting region (residues 185-200), and finally Domain III (residues 201-303) that forms a cluster of helices 17. The substrate binding site is located in the cleft between domain I and domain II 17. Compared to WA1, we found only three mutations in Mpro, namely a K90R mutation in the B1.351 (Beta) strain and a L205V mutation in the P2 (Zeta) strain, and P132H mutation in B.1.1.529 (Omicron), none of the mutated residues were part of the substrate binding site (FIG. 1A). In contrast, the mutation rate was significantly higher in S protein with 78 mutations (substitution, deletion and insertion) (FIG. 1B, FIG. 3A, Table 1) 18. FIG. 3B shows inter-molecular interactions of ZINC000085591448, ZINC000097972782, ZINC000005462364, ZINC000644163977, ZINC000008077555, ZINC000230464020, ZINC000006623878, ZINC000085876900 and ZINC000085879857 and SARS-CoV-2 M^{pro}. Together, this comparison indicates that the substrate binding site of Mpro is more conserved among all known human SARS coronaviruses, thus making this an ideal anti-viral drug target.

TABLE 1
Mutations of Mpro and Spike protein across SARS-CoV-2 VOCs.

| | | Substitution | | | | |
|---|---|---|---|---|---|---|
| COVID 19 | Spike Substitution | Numb | Deletion | DeletionNumb | Insertion | InsertionNumb |
| B.1.1.7 | N501Y, A570D, D614G, | 7 | 69del, 70del, | 3 | NA | 0 |
| (Alpha) | P681H, T716I, S982A, | | 144del | | | |
| | D1118H | | | | | |
| B.1.351(Beta) | L18F, D80A, D215G, | 8 | 242del, 243del, | 3 | NA | 0 |
| | K417N, E484K, N501Y, | | 241del | | | |
| | D614G, A701V | | | | | |
| B.1.427(Epsilon) | S13I, Y145H, W152C, | 5 | NA | 0 | NA | 0 |
| | L452R, D614G | | | | | |
| B.1.429(Epsilon) | S13I, W152C, L452R, | 4 | NA | 0 | NA | 0 |
| | D614G | | | | | |
| B.1.525(Eta) | Q52R, A67V, E484K, | 6 | 69del, 70del, | 3 | NA | 0 |
| | D614G, Q677H, F888L | | 145del, | | | |
| B.1.526(Iota) | L5F, T95I, D253G, | 6 | NA | 0 | NA | 0 |
| | E484K, D614G, A701V | | | | | |
| B.1.526.1 | F156S, L452R, D614G, | 4 | 145del | 1 | NA | 0 |
| | D950H, | | | | | |
| B.1.617.1(Kappa) | E154K, L452R, E484Q, | 7 | NA | 0 | NA | 0 |
| | D614G, P681R, Q1071H, | | | | | |
| | H1101D | | | | | |
| B.1.617.2(Delta) | T19R, R158G, L452R, | 7 | 156del, 157del | 2 | NA | 0 |
| | T478K, D614G, P681R, | | | | | |
| | D950N, | | | | | |
| P.1(Gamma) | L18F, T20N, P26S, | 12 | NA | 0 | NA | 0 |
| | D138Y, R190S, K417T, | | | | | |
| | E484K, N501Y, D614G, | | | | | |
| | H655Y, T1027I, V1176F, | | | | | |
| P.2(Zeta) | E484K, D614G, V1176F | 3 | NA | 0 | NA | 0 |
| B.1.1.529(Omicron) | A67V, T95I, G142D, | 33 | 69del, 70del, | 5 | ins213R, | 2 |
| | N211L, L212V, V215P, | | 143del, 144del, | | ins214E | |
| | R216E, G339D, S371L, | | 145del, | | | |
| | S373P, S375F, K417N, | | | | | |
| | N440K, G446S, S477N, | | | | | |
| | T478K, E484A, Q493R, | | | | | |
| | G496S, Q498R, N501Y, | | | | | |
| | Y505H, T547K, D614G, | | | | | |
| | H655Y, N679K, P681H, | | | | | |
| | N764K, D796Y, N856K, | | | | | |
| | Q954H, N969K, L981F | | | | | |

### Integrative In-Silico Screening Identified Novel Candidate Inhibitors of Mpro.

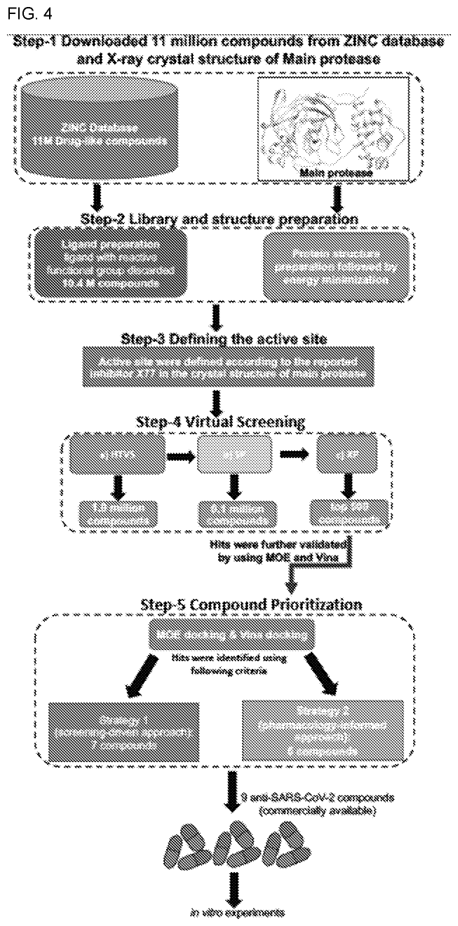

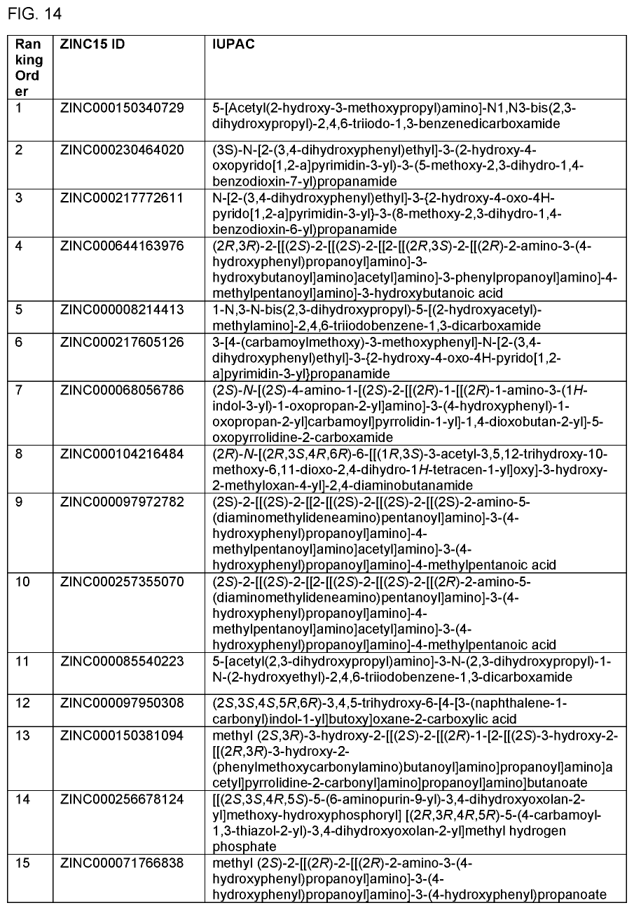

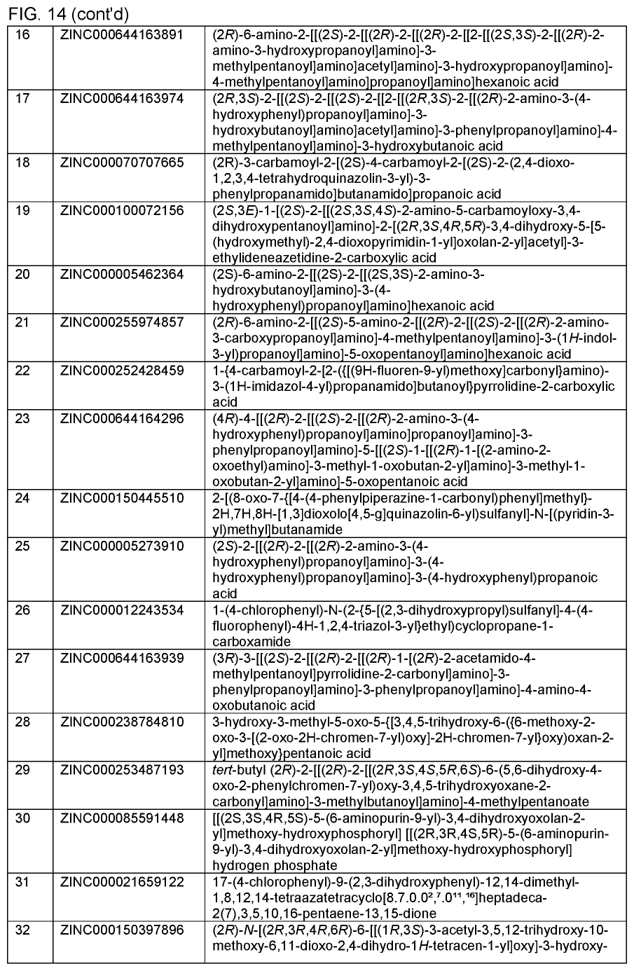

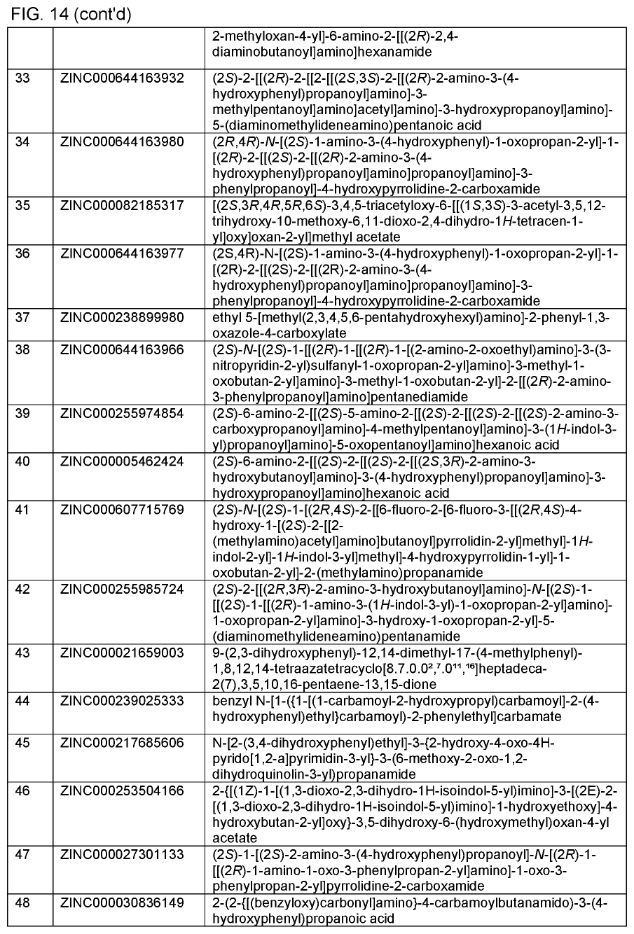

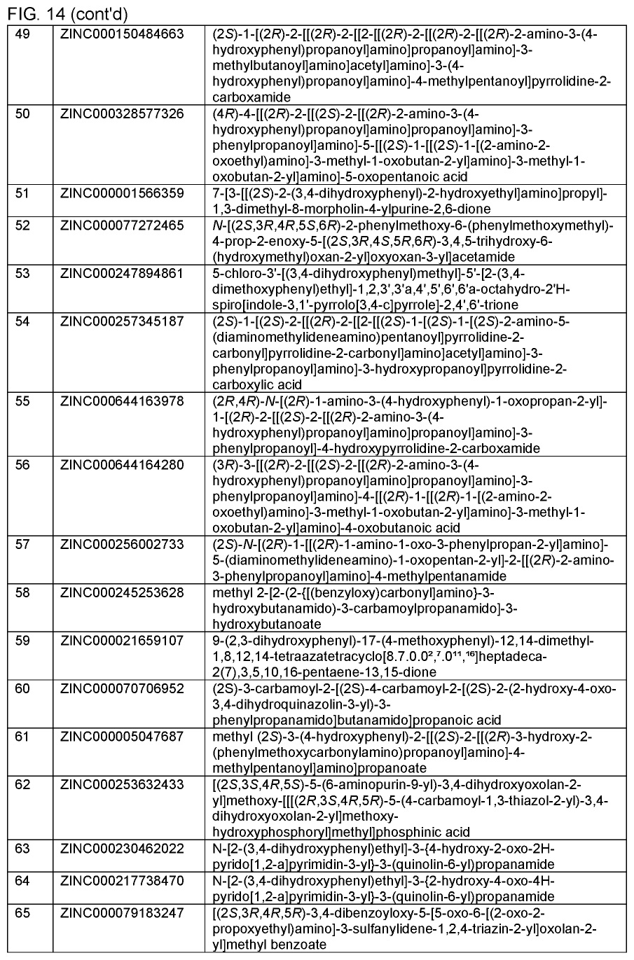

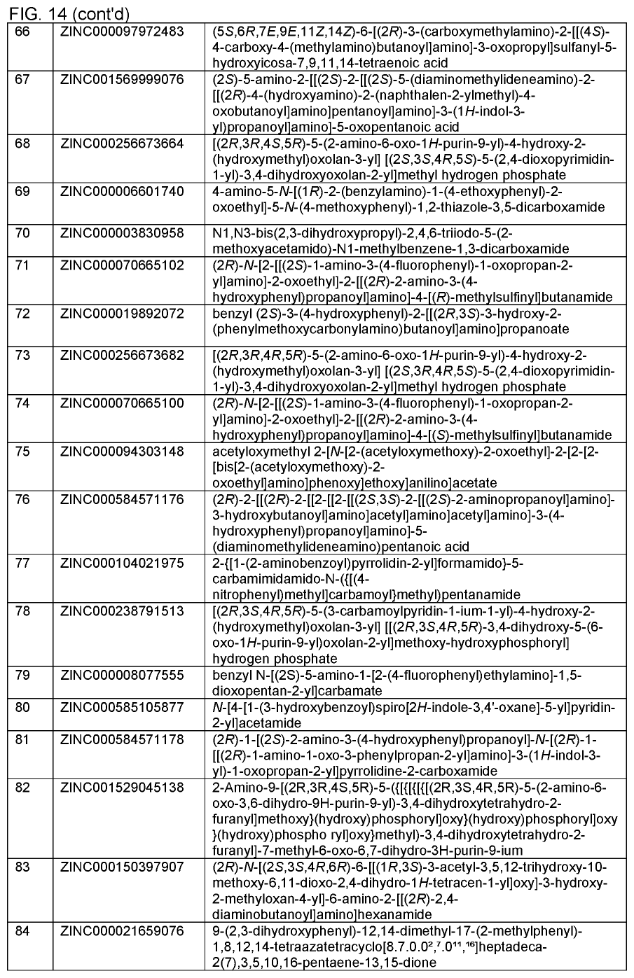

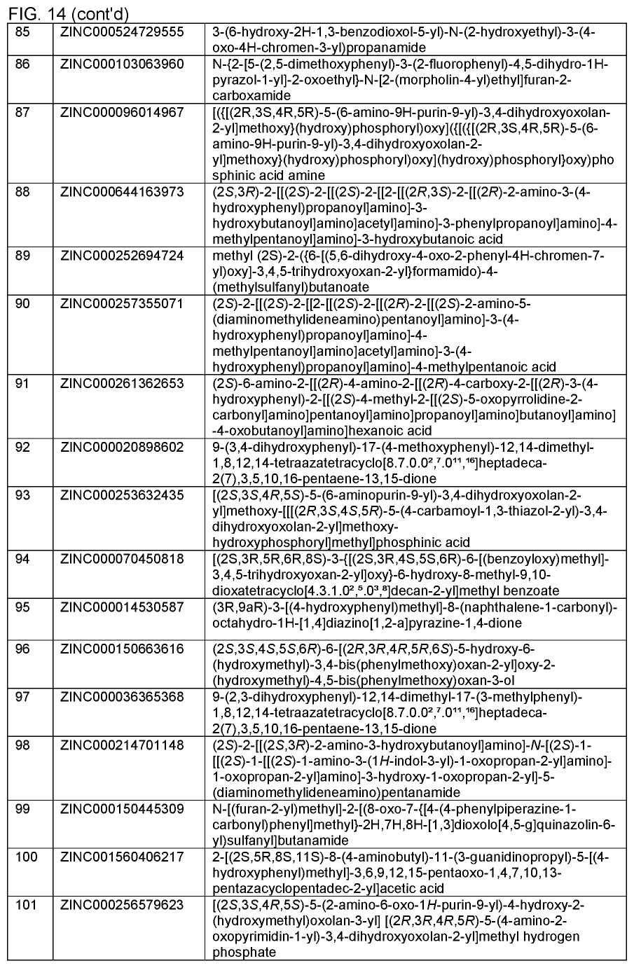

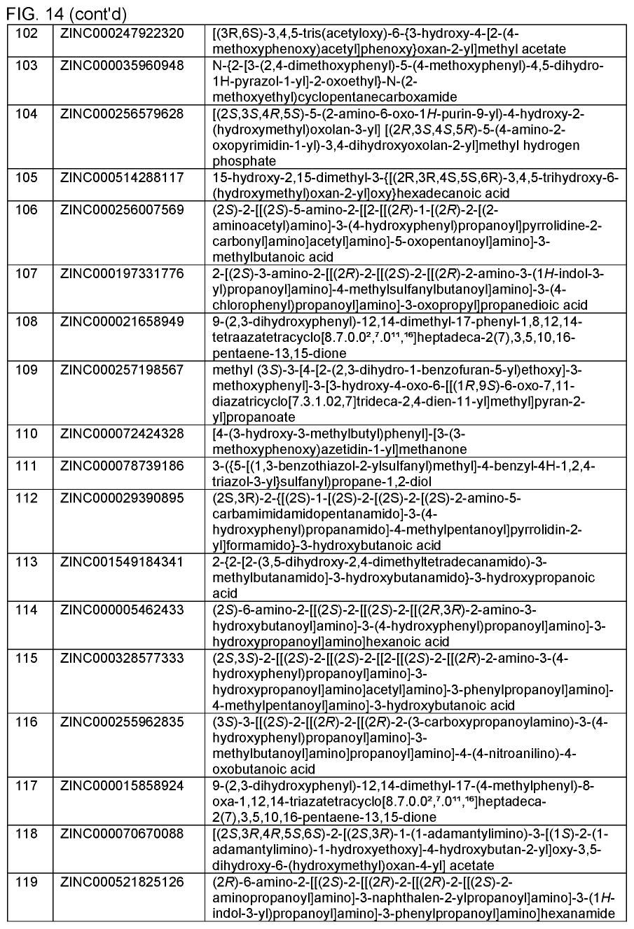

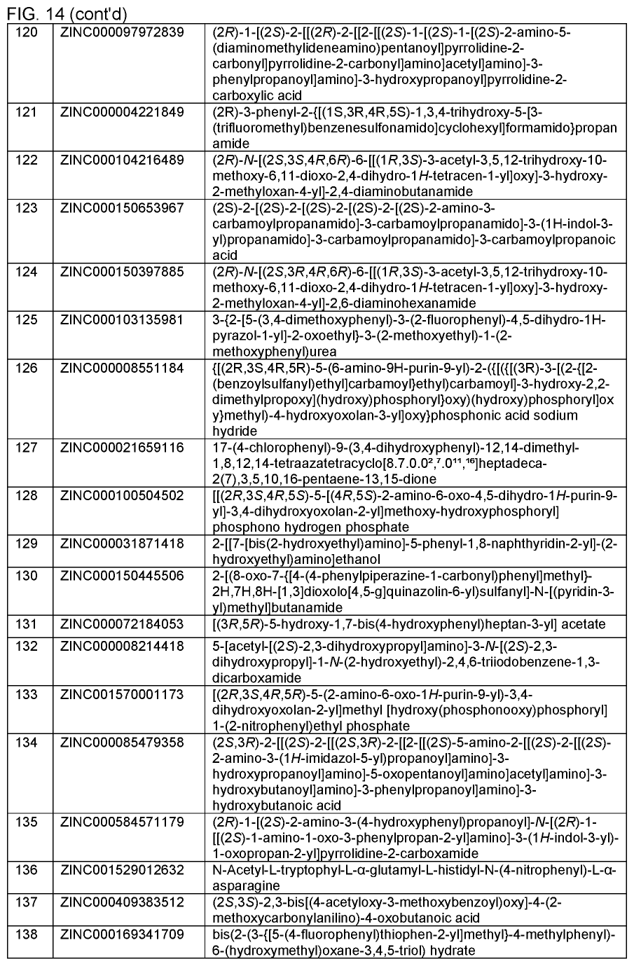

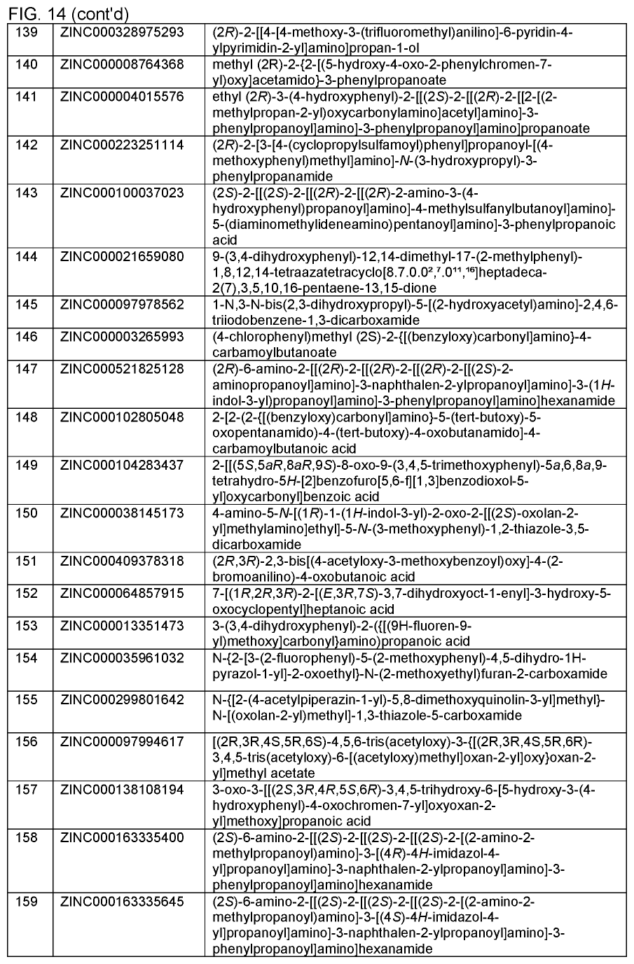

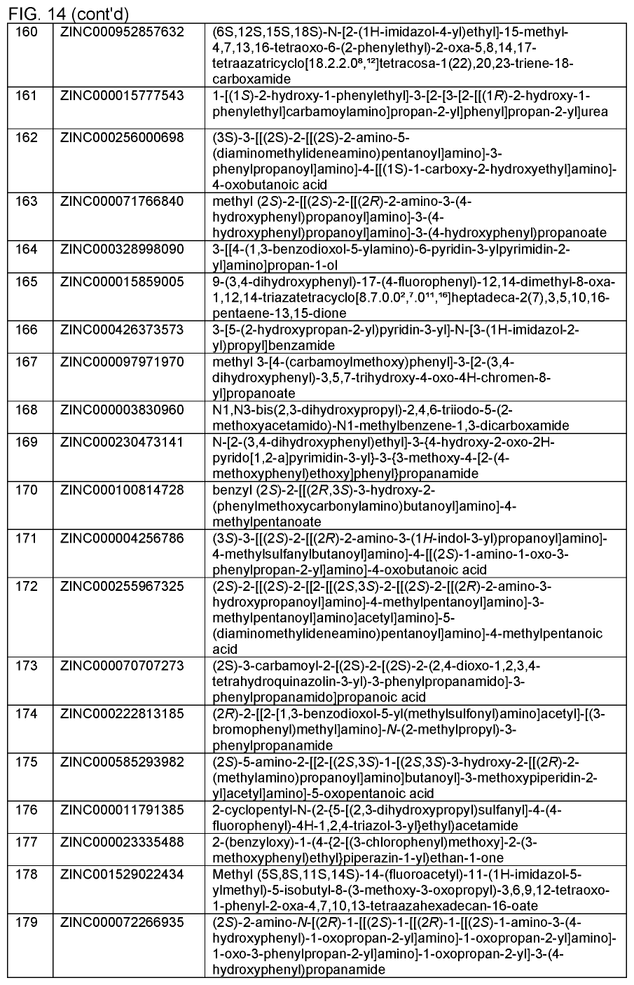

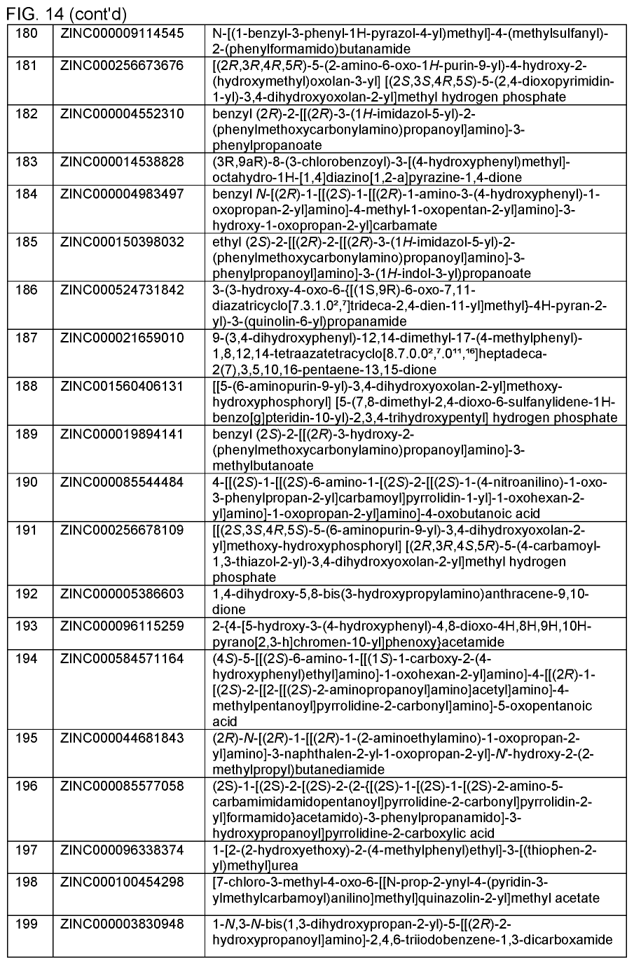

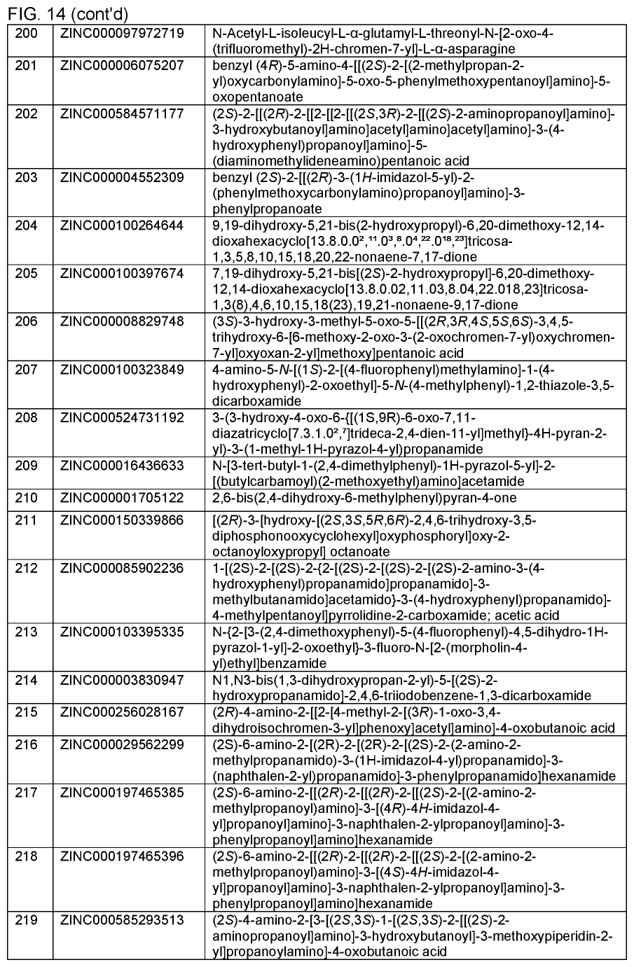

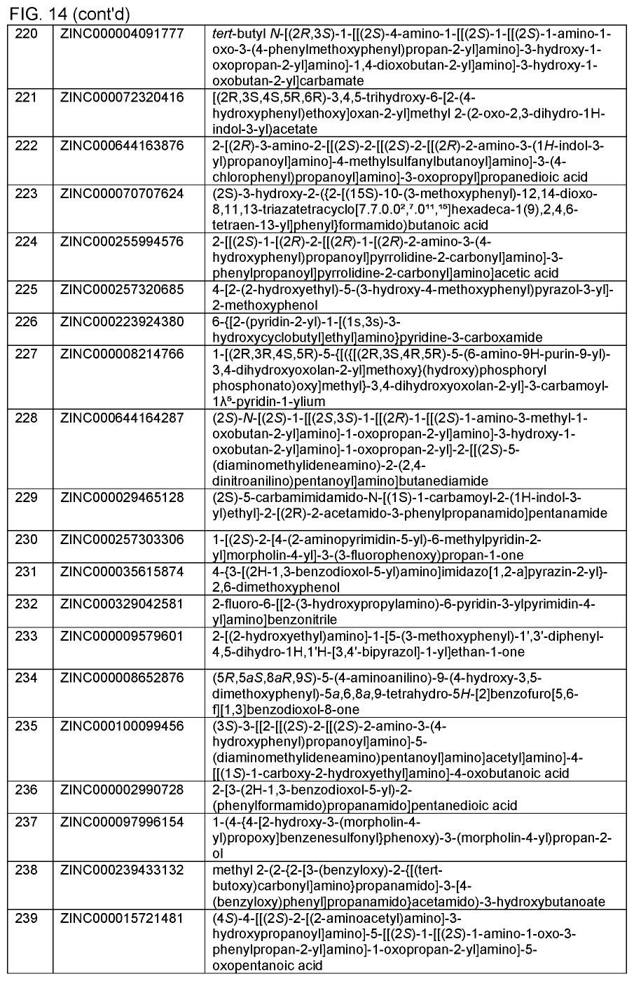

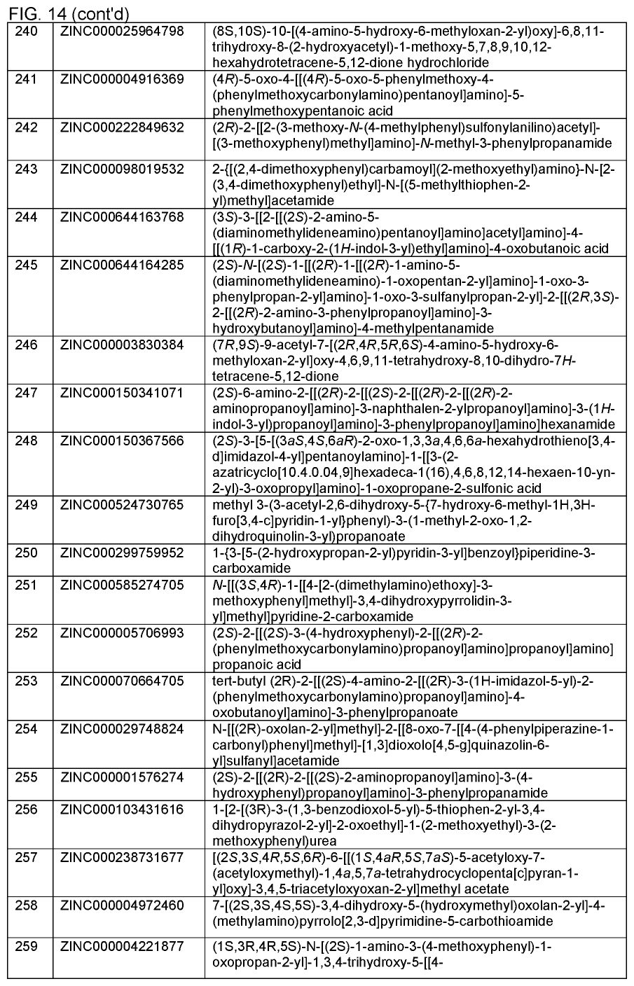

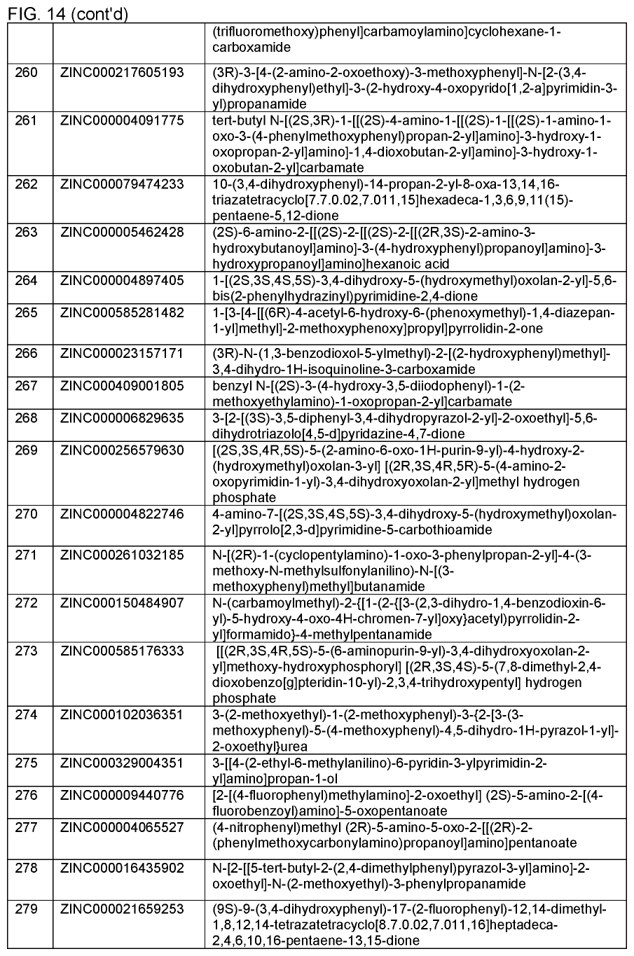

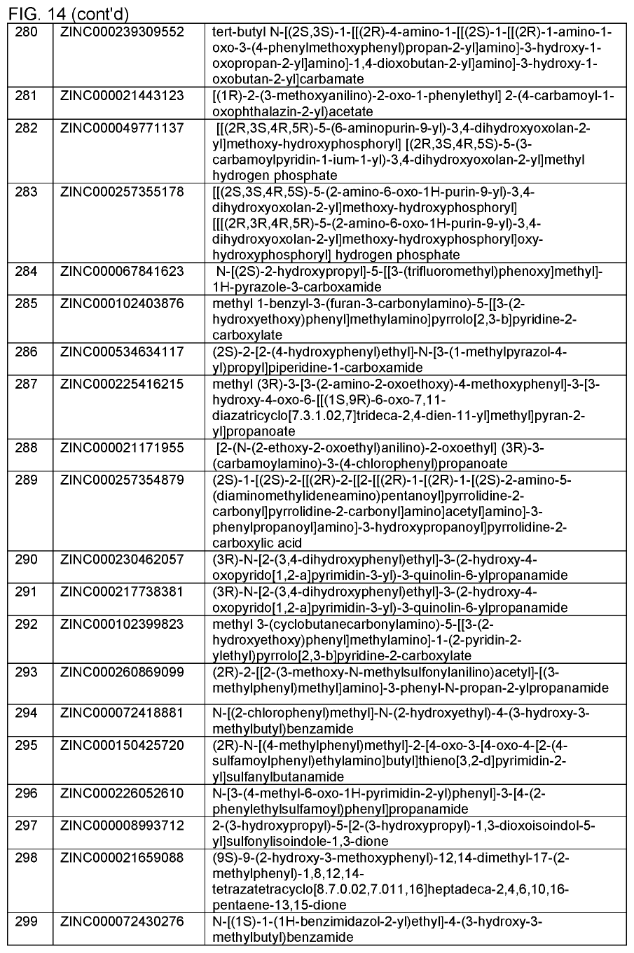

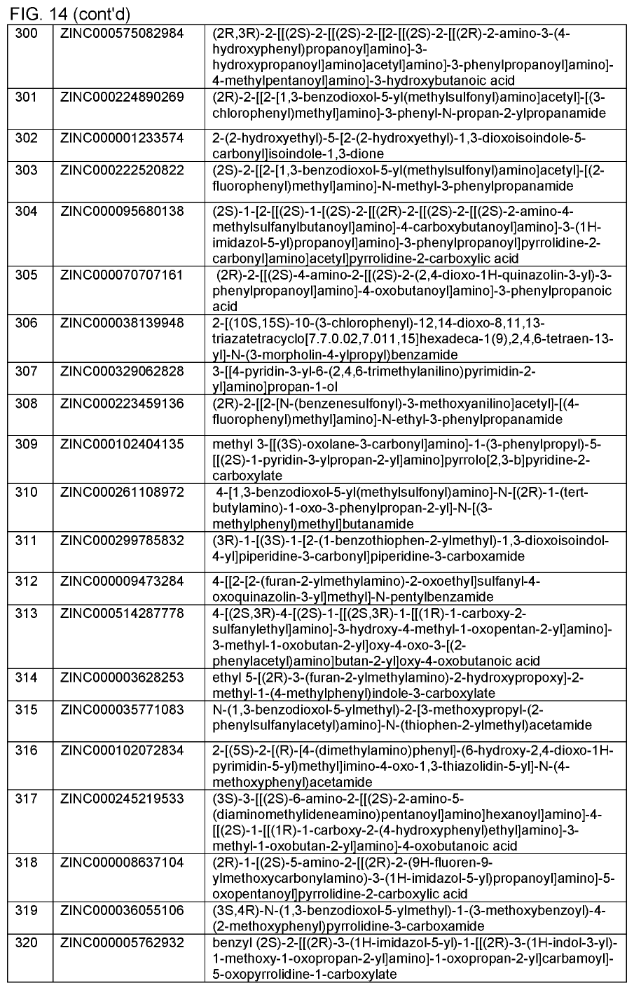

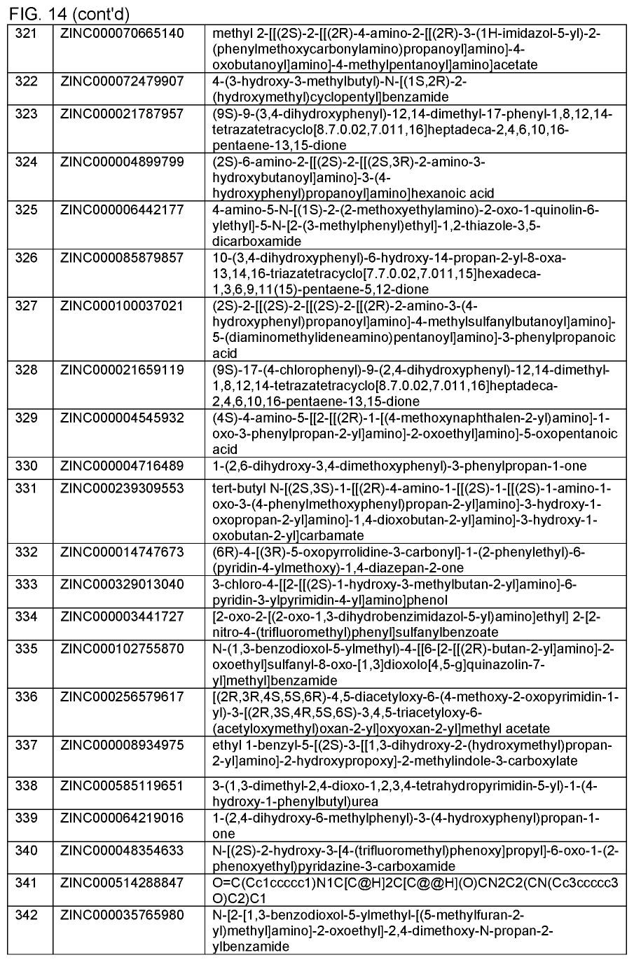

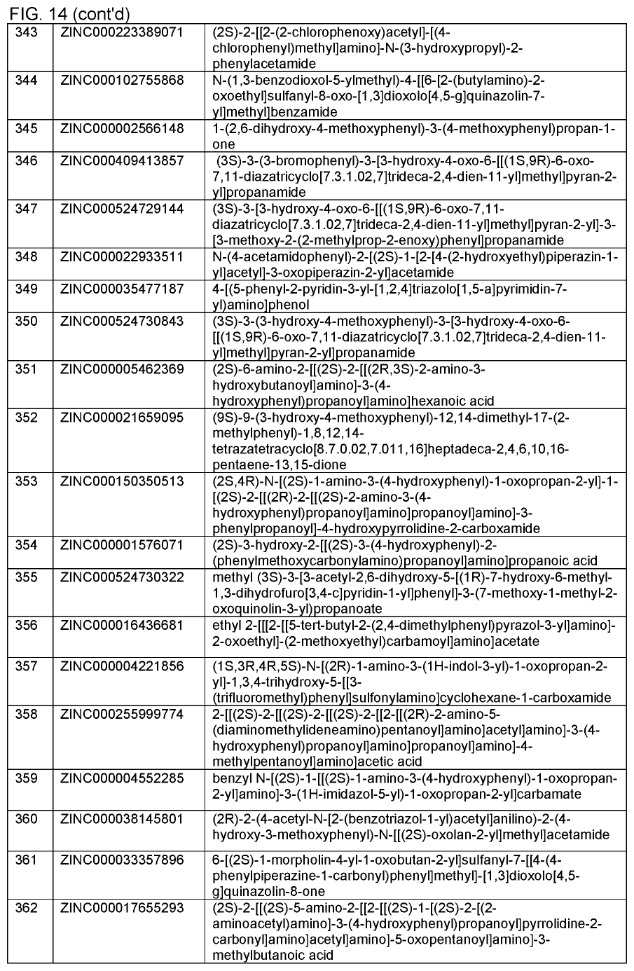

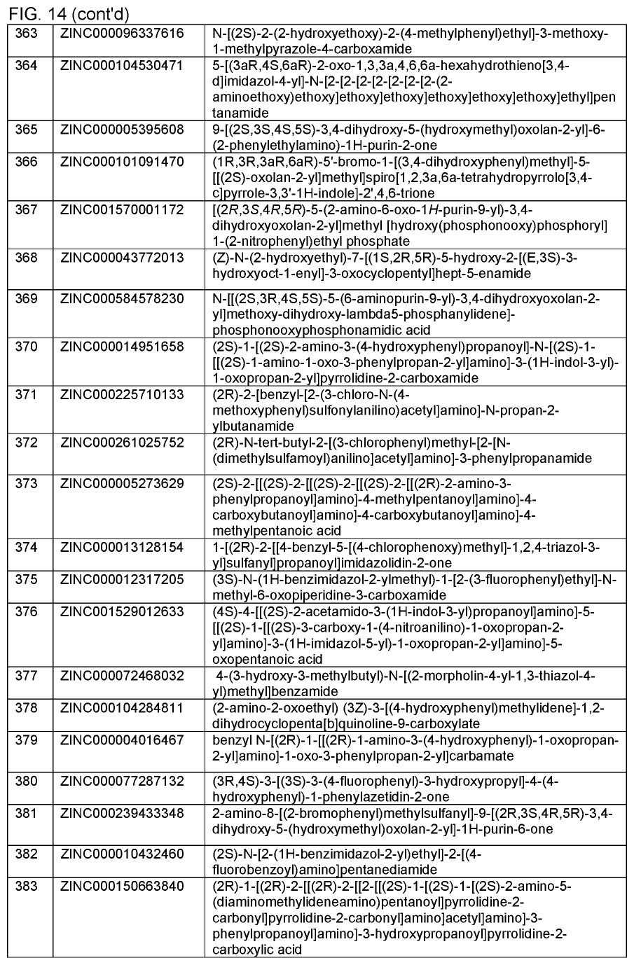

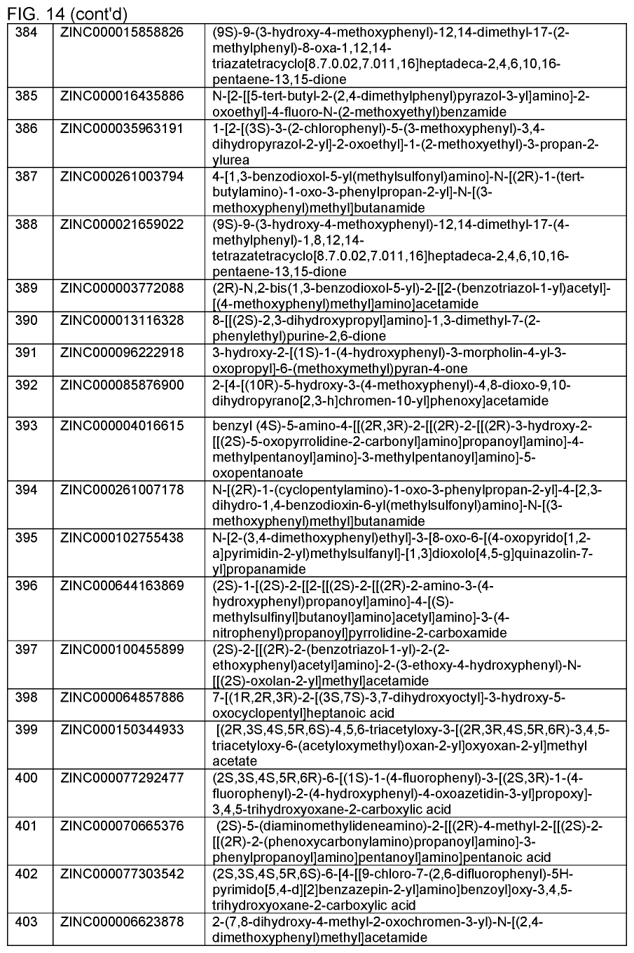

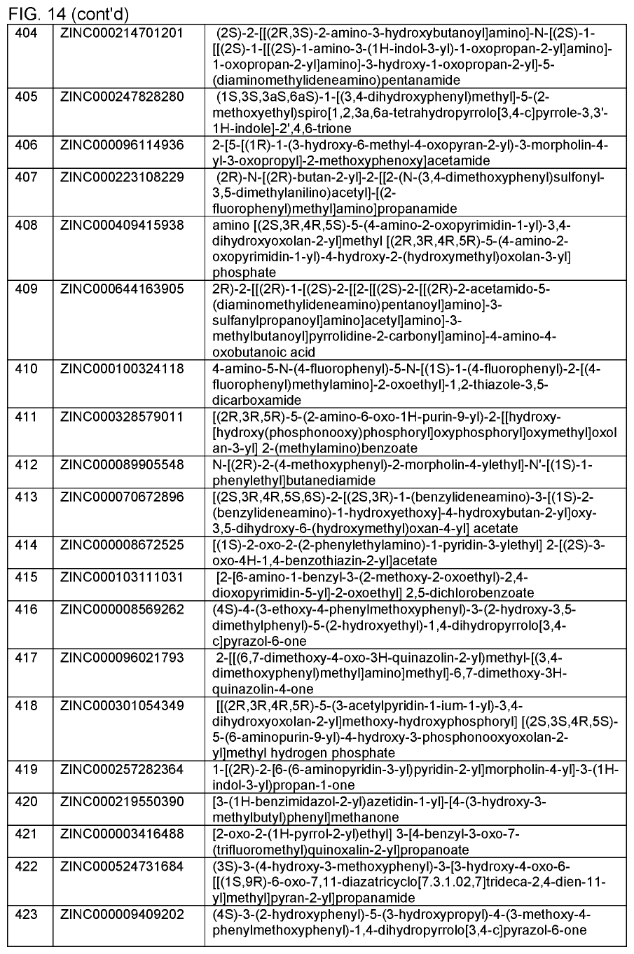

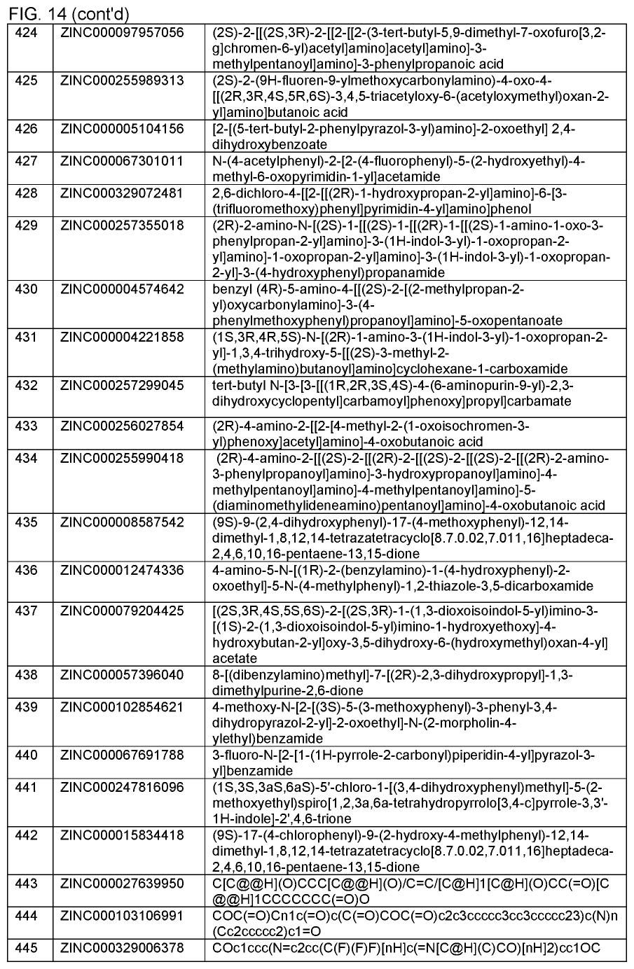

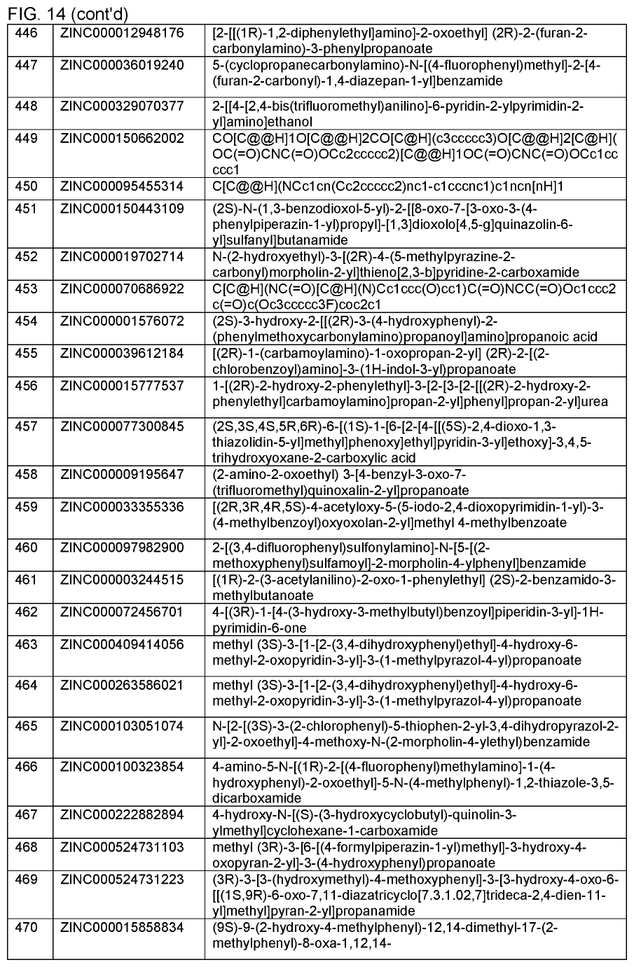

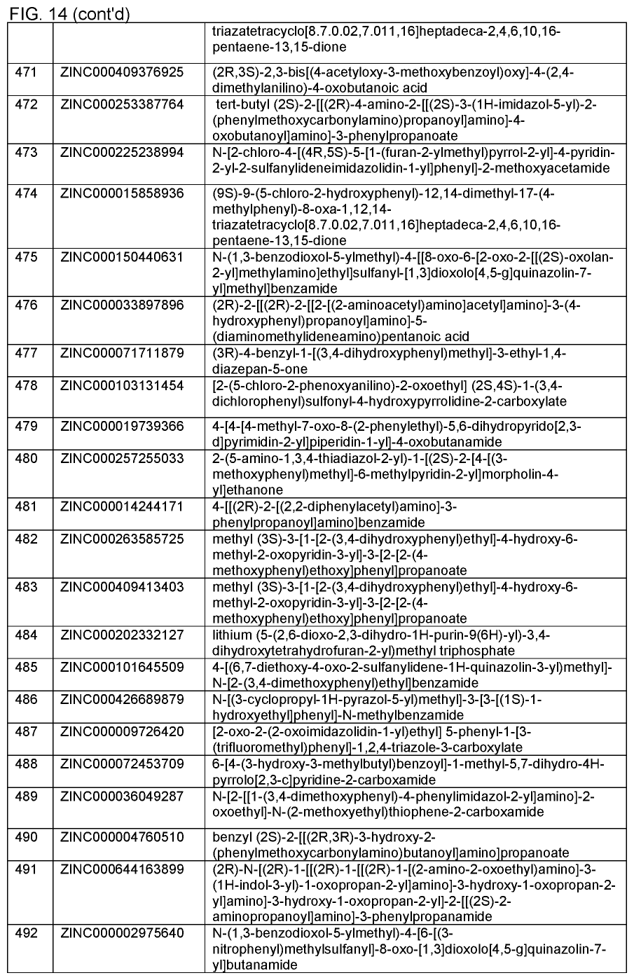

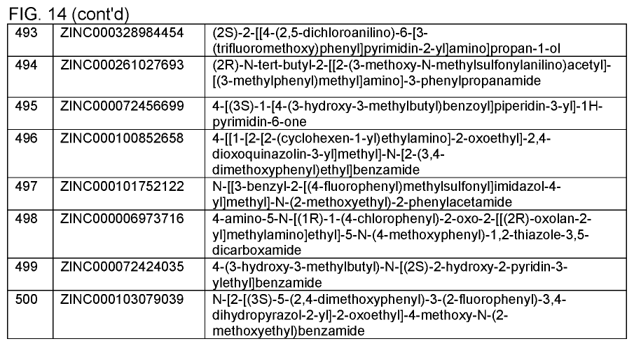

Structure-based virtual screening is a fast and powerful method for lead compound discovery. We developed an integrative approach for in-silico screening and prioritization (FIG. 4) compounds targeting Mpro of SARS-CoV-2. In step 1, we downloaded 11 million Drug-Like In-Stock 3D small molecules from ZINC database¹⁹ and the X-ray crystal structure of Mpro from X77 (https://www.rcsb.org/structure/6W63). In step 2, we prepared the X-ray crystal structure of Mpro with the Protein Preparation Wizard in Maestro from Schrödinger and prepared ligand and removed compounds with reactive functional groups to obtain 10.4 M compounds. In step 3, we defined the binding pockets of Mpro, based on the reported inhibitor X77 (https://www.resb.org/structure/6W63). In step 4, we performed virtual screening with Schrödinger²⁰ to select top-500 lead compounds. In step 5, we prioritize the top-ranked 500 lead compounds by integrating two complementary strategies, which lead to the selection of total 9 lead compounds (Table 2) for in-vitro testing. FIG. 14 shows the top-ranked 500 lead compounds.

TABLE 2
Summary of in-silico screening results for the prioritized 9 lead inhibitors of Mpro of SARS-CoV-2.

| ZINC15 ID | Compound Name |
|---|---|
| ZINC000085591448 | [[(2S,3S,4R,5S)-5-(6-aminopurin-9-yl)-3,4-dihydroxyoxolan-2- |
| | yl]methoxy-hydroxyphosphoryl] [[(2R,3R,4S,5R)-5-(6-aminopurin- |
| | 9-yl)-3,4-dihydroxyoxolan-2-yl]methoxy-hydroxyphosphoryl] |
| | hydrogen phosphate |
| ZINC000097972782 | (2S)-2-[[(2S)-2-[[2-[[(2S)-2-[[(2S)-2-[[(2S)-2-amino-5- |
| | (diaminomethylideneamino)pentanoyl]amino]-3-(4- |
| | hydroxyphenyl)propanoyl]amino]-4-methylpentanoyl]amino]acetyl]amino]- |
| | 3-(4-hydroxyphenyl)propanoyl]amino]-4-methylpentanoic acid |
| ZINC000005462364 | (2S)-6-amino-2-[[(2S)-2-[[(2S,3S)-2-amino-3-hydroxybutanoyl]amino]- |
| | 3-(4-hydroxyphenyl)propanoyl]amino]hexanoic acid |
| ZINC000644163977 | (2S,4R)-N-[(2S)-1-amino-3-(4-hydroxyphenyl)-1-oxopropan-2-yl]-1-[(2R)- |
| | 2-[[(2S)-2-[[(2R)-2-amino-3-(4-hydroxyphenyl)propanoyl]amino]propanoyl]amino]- |
| | 3-phenylpropanoyl]-4-hydroxypyrrolidine-2-carboxamide |
| ZINC000008077555 | benzyl N-[(2S)-5-amino-1-[2-(4-fluorophenyl)ethylamino]-1,5-dioxopentan-2- |
| | yl]carbamate |
| ZINC000230464020 | (3S)-N-[2-(3,4-dihydroxyphenyl)ethyl]-3-(2-hydroxy-4-oxopyrido[1,2- |
| | a]pyrimidin-3-yl)-3-(5-methoxy-2,3-dihydro-1,4-benzodioxin-7-yl)propanamide |
| ZINC000006623878 | 2-(7,8-dihydroxy-4-methyl-2-oxochromen-3-yl)-N-[(2,4- |
| | dimethoxyphenyl)methyl]acetamide |
| ZINC000085876900 | 2-[4-[(10R)-5-hydroxy-3-(4-methoxyphenyl)-4,8-dioxo-9,10-dihydropyrano[2,3- |
| | h]chromen-10-yl]phenoxy]acetamide |
| ZINC000085879857 | 10-(3,4-dihydroxyphenyl)-6-hydroxy-14-propan-2-yl-8-oxa-13,14,16- |
| | triazatetracyclo[7.7.0.02,7.011,15]hexadeca-1,3,6,9,11(15)-pentaene-5,12-dione |

| | Glide score | Vina score | MOE score | Molecule |
|---|---|---|---|---|
| ZINC15 ID | in kcal/mol | in kcal/mol | in kcal/mol | Weight |
| ZINC000085591448 | −10.52 | −7.77 | −13.04 | 753.387 |
| ZINC000097972782 | −11 | −6.8 | −12.58 | 784.933 |
| ZINC000005462364 | −10.67 | −6.67 | −13.48 | 411.477 |
| ZINC000644163977 | −10.32 | −8.53 | −13.27 | 675.76 |
| ZINC000008077555 | −9.9 | −7.3 | −11.7 | 401.437 |
| ZINC000230464020 | −11.79 | −8.73 | −16.72 | 533.535 |
| ZINC000006623878 | −8.99 | −7.77 | −12.4 | 398.39 |
| ZINC000085876900 | −9 | −9.97 | −13.2 | 486.455 |
| ZINC000085879857 | −9.12 | −8.7 | −16 | 407.381 |

| ZINC15 ID | cLogS | Druglikeness | LE | Mutagenic | Tumorigenic |
|---|---|---|---|---|---|
| ZINC000085591448 | −1.296 | −38.717 | 0.17143 | none | none |
| ZINC000097972782 | −4.541 | 2.873 | 0.14956 | none | none |
| ZINC000005462364 | −1.785 | −0.47956 | 0.30208 | none | none |
| ZINC000644163977 | −3.5 | 6.527 | 0.17275 | none | none |
| ZINC000008077555 | −4.074 | −11.33 | 0.30259 | none | none |
| ZINC000230464020 | −4.817 | −1.7465 | 0.22066 | none | none |
| ZINC000006623878 | −4.177 | 3.7173 | 0.30275 | none | none |
| ZINC000085876900 | −4.974 | 0.16848 | 0.24057 | none | none |
| ZINC000085879857 | −5.347 | 2.7625 | 0.29221 | none | none |

| | | | | Total Polar |
|---|---|---|---|---|
| | | Natural | | Surface Area |
| ZINC15 ID | Irritant | compound | LogP | (TPSA) |
| ZINC000085591448 | none | No | −2.563 | 395 |
| ZINC000097972782 | none | No | −0.294 | 317 |
| ZINC000005462364 | none | No | −1.174 | 194 |
| ZINC000644163977 | none | No | −0.626 | 239 |
| ZINC000008077555 | none | No | 2.045 | 110 |
| ZINC000230464020 | none | No | 2.472 | 154 |
| ZINC000006623878 | none | Yes | 2.389 | 118 |
| ZINC000085876900 | none | Yes | 3.479 | 138 |
| ZINC000085879857 | none | Yes | 3.694 | 147 |

The identified 9 lead compounds (ZINC000085591448, ZINC000097972782, ZINC000005462364, ZINC000644163977, ZINC000008077555, ZINC000230464020, ZINC000006623878, ZINC000085876900 and ZINC000085879857) showed higher binding affinity with Mpro (Table 2). From the molecular interaction analysis of docked complexes, we observed that all the 9 lead compounds show hydrogen bonding interactions and other potential hydrophobic or hydrophilic interactions with Mpro were bound to the same binding site residues, Thr25, Leu27, His41, Cys44, His164, Asp187, Arg188, Cys145, Met49, and Met165 were the common interacting residues between Mpro, and 9 lead compounds suggested the crucial role of these in stabilizing the Mpro-ligand complex. The structure, hydrogen bond and hydrophobic interactions obtained for the 9 lead compounds are shown in FIG. 6. Importantly, to demonstrate our 9 lead molecules are robust against VOCs of SARS-CoV-2, we built Mpro structures with K90R mutation in the B1.351 (Beta), L205V mutation in the P2 (Zeta), and P132H mutation in B.1.1.529 (Omicron), by using UCSF Chimera²¹ followed by the Protein Preparation Wizard in the Schrodinger package and validated all compounds by docking them to these three mutant structures of Mpro. The docking results showed very similar binding affinities of these lead compounds to the mutant structures compared to wild-type Mpro (Table 3), suggesting that these lead compounds have the potential for the robust inhibition of the currently known VOCs of SARS-CoV-2.

TABLE 3
Docking scores for 4 validated inhibitors with Mpro from SARS-CoV2
WA1 and mutated variants: B.1.351, B.1.1529 and P.2 variants.

| | | Docking score | | Docking score |
|---|---|---|---|---|
| | Docking score | with Mpro | Docking score | with Mpro |
| | with Mpro WA1 | B.1.351 | with Mpro P.2 | B.1.1.529 |
| Compounds | (kcal/mol) | (kcal/mol) | (kcal/mol) | (kcal/mol) |
| ZINC000230464020 | −11.79 | −11.854 | −11.859 | −9.34 |
| ZINC000008077555 | −9.9 | −9.246 | −8.046 | −7.558 |
| ZINC000085876900 | −9 | −8.946 | −9.083 | −6.661 |
| ZINC000006623878 | −8.99 | −9.057 | −8.672 | −6.82 |

### In Vitro Validation of Inhibitors of SARS-CoV-2 Infection.

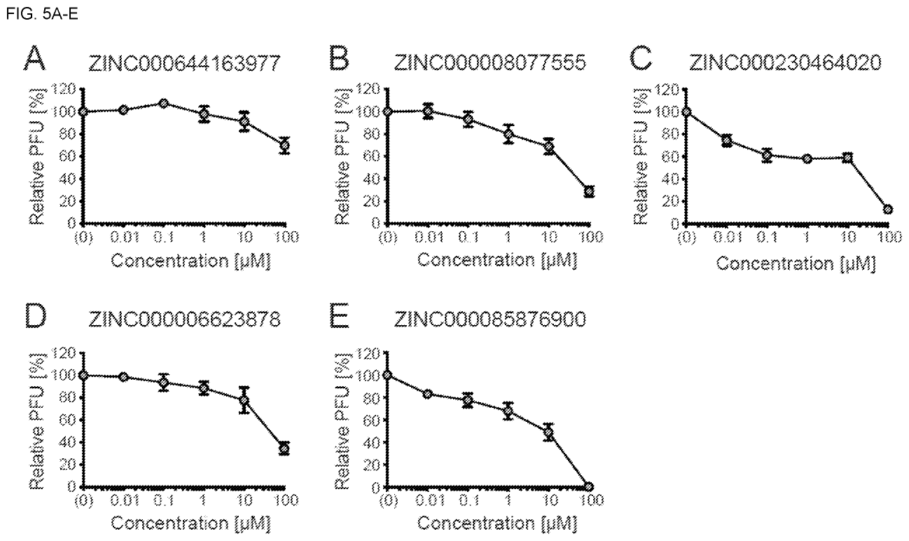

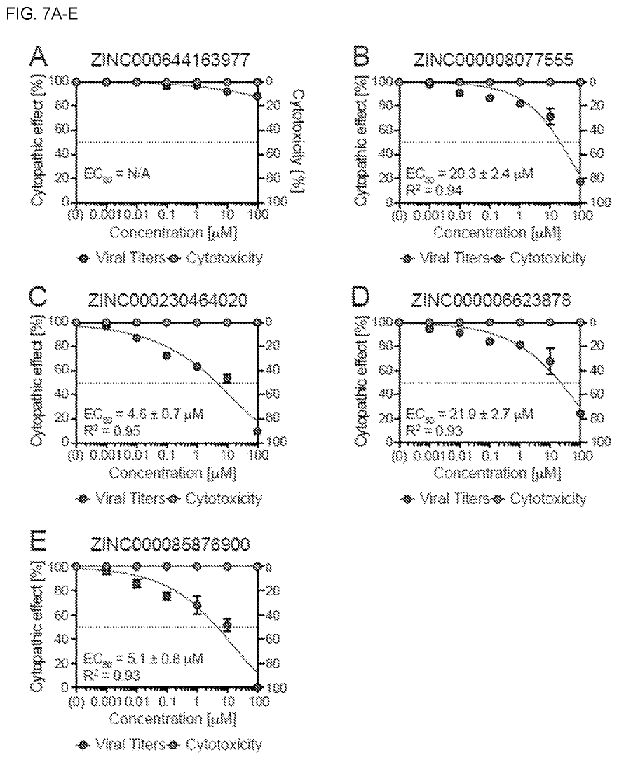

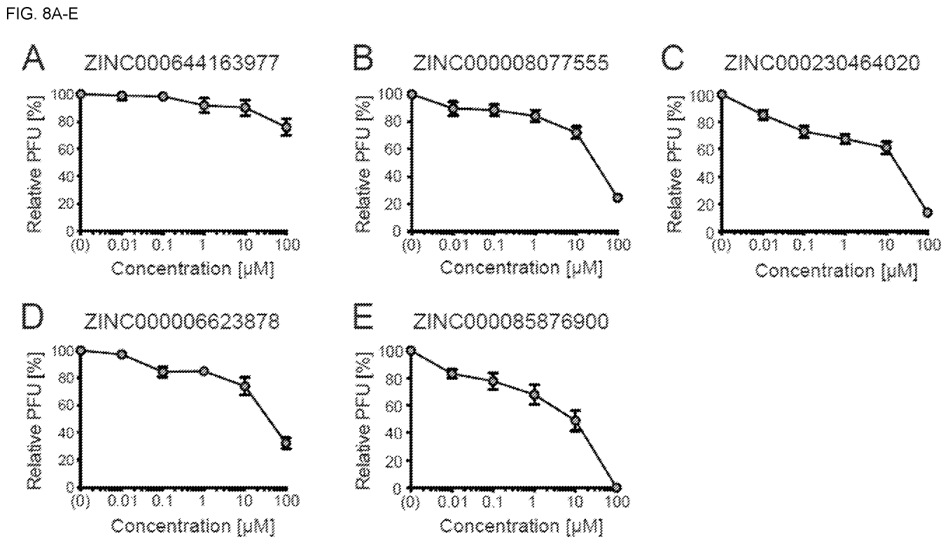

We tested the ability of the selected 9 lead compounds to suppress SARS-CoV-2 infection in two experimental settings. Initially, we infected Vero cells with SARS-CoV-2 immediately after the addition of increasing concentrations of the lead compounds and measured the number of infected cells by viral reduction plaque assay 4 days later. Cells infected in the absence of the compounds served as positive control, whereas uninfected cells cultured in the presence of increasing concentrations of the compounds served as control for cytotoxicity. We observed a significant drop in the number of infected cells compared to positive control for 4 of the 9 lead compounds (ZINC000008077555, ZINC000230464020, ZINC000006623878, and ZINC000085876900), whereas the remaining compounds showed only modest (ZINC000644163977) or no inhibitory activity (Table 4, FIG. 5). In the second experiment, we further validated the 4 active compounds. We measured the cytopathic effect (CPE) on Vero cells 4 days after infection with SARS-CoV-2 in the presence of increasing concentrations of the compounds, starting at the time of infection. We also included ZINC000644163977, which showed modest activity in the initial experiments as a negative control. As Vero cells are known to express high-levels of the ABC transporter P-gp, we also added the P-gp inhibitor CP-100356 to these experiments to limit the potential extracellular export of the compounds. In parallel, we also determined the cytotoxicity of these compounds over the same dose range. These experiments revealed that ZINC000644163977 showed negligible activity (FIG. 7A), whereas ZINC000008077555, ZINC000230464020, ZINC000006623878, and ZINC000085876900 inhibited virus-induced CPE with an EC50 between 4.6-22.6 μM (FIG. 7B-E). Compared to the initial experiments, P-gp inhibition had only a minimal effect on the activity of these compounds (FIG. 7A-E, FIG. 5). Importantly, none of the compounds showed any sign of cytotoxicity (FIG. 7A-E). Finally, to test whether the choice of cell line influenced the drug-mediated inhibition of viral replication, we tested their impact on SARS-CoV-2-replication also in human lung-derived Calu-3 cells by plaque reduction assay and showed that the 4 lead compounds are also active in human cells (FIG. 8). Overall, these experiments confirmed 4 out of the total 9 lead compounds significantly suppressed SARS-CoV-2 infection in vitro.

TABLE 4
Summary of inhibitory activity of SARS-CoV2 for the prioritized
9 lead inhibitors by in-vitro experiments.

| | | Inhibition |
|---|---|---|
| ZINC15 ID | Compound Name | of Infection |
| ZINC000085591448 | [[(2S,3S,4R,5S)-5-(6-aminopurin-9-yl)-3,4-dihydroxyoxolan- | − |
| | 2-yl]methoxy-hydroxyphosphoryl] [[(2R,3R,4S,5R)-5-(6- | |
| | aminopurin-9-yl)-3,4-dihydroxyoxolan-2-yl]methoxy- | |
| | hydroxyphosphoryl] hydrogen phosphate | |
| ZINC000097972782 | (2S)-2-[[(2S)-2-[[2-[[(2S)-2-[(2S)-2-[[(2S)-2-amino-5- | − |
| | (diaminomethylideneamino)pentanoyl]amino]-3-(4- | |
| | hydroxyphenyl)propanoyl]amino]-4- | |
| | methylpentanoyl]amino]acetyl]amino]-3-(4- | |
| | hydroxyphenyl)propanoyl]amino]-4-methylpentanoic acid | |
| ZINC000005462364 | (2S)-6-amino-2-[[(2S)-2-[[(2S,3S)-2-amino-3- | − |
| | hydroxybutanoyl]amino]-3-(4- | |
| | hydroxyphenyl)propanoyl]amino]hexanoic acid | |
| ZINC000644163977 | (2S,4R)-N-[(2S)-1-amino-3-(4-hydroxyphenyl)-1-oxopropan- | +/− |
| | 2-yl]-1-[(2R)-2-[[(2S)-2-[[(2R)-2-amino-3-(4- | |
| | hydroxyphenyl)propanoyl]amino]propanoyl]amino]-3- | |
| | phenylpropanoyl]-4-hydroxypyrrolidine-2-carboxamide | |
| ZINC000008077555 | benzyl N-[(2S)-5-amino-1-[2-(4-fluorophenyl)ethylamino]- | +/− |
| | 1,5-dioxopentan-2-yl]carbamate | |
| ZINC000230464020 | (3S)-N-[2-(3,4-dihydroxyphenyl)ethyl]-3-(2-hydroxy-4- | + |
| | oxopyrido[1,2-a]pyrimidin-3-yl)-3-(5-methoxy-2,3- | |
| | dihydro-1,4-benzodioxin-7-yl)propanamide | |
| ZINC000006623878 | 2-(7,8-dihydroxy-4-methyl-2-oxochromen-3-yl)-N-[(2,4- | ++ |
| | dimethoxyphenyl)methyl]acetamide | |
| ZINC000085876900 | 2-[4-[(10R)-5-hydroxy-3-(4-methoxyphenyl)-4,8-dioxo- | + |
| | 9,10-dihydropyrano[2,3-h]chromen-10- | |
| | yl]phenoxy]acetamide | |
| ZINC000085879857 | 10-(3,4-dihydroxyphenyl)-6-hydroxy-14-propan-2-yl-8-oxa- | − |
| | 13,14,16-triazatetracyclo[7.7.0.02,7.011,15]hexadeca- | |
| | 1,3,6,9,11(15)-pentaene-5,12-dione | |

Note
++ Validated strong inhibitor
+/− Validated modest inhibitor
− Validated no effect

### SARS-CoV-2 Replication is Specifically Inhibited by Selected Lead Molecules.

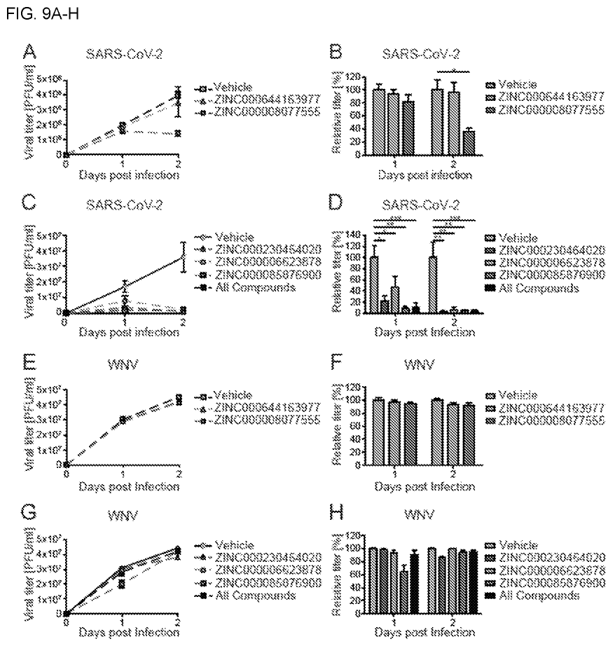

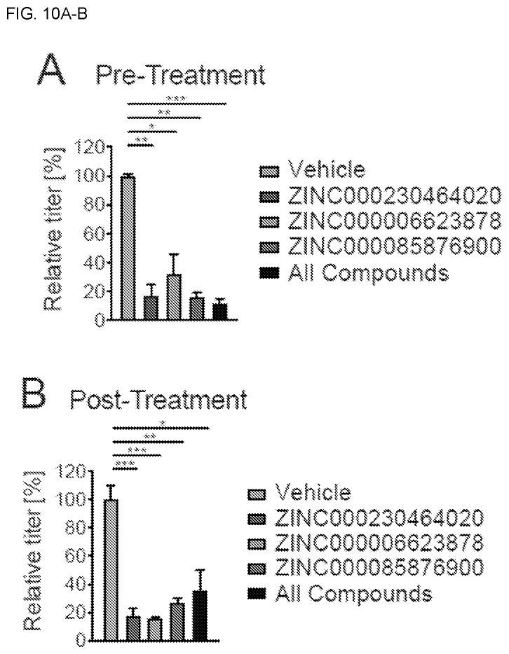

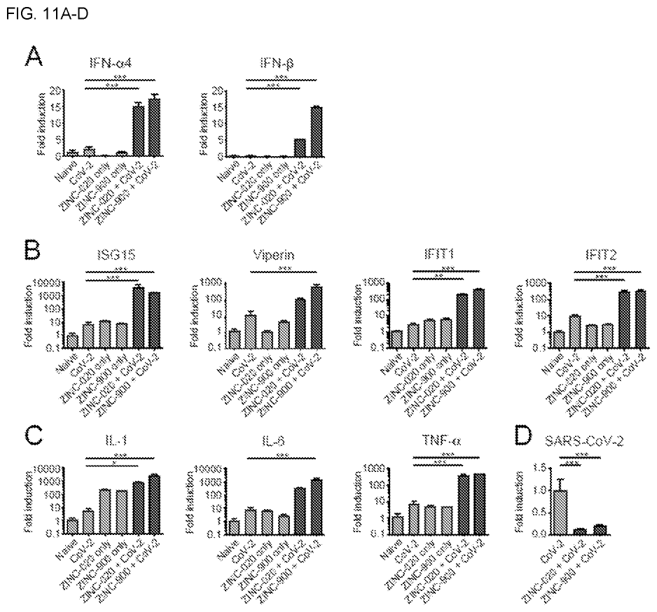

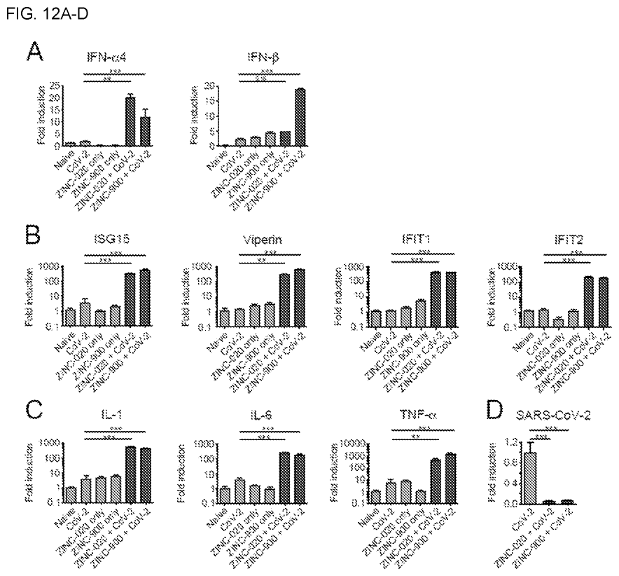

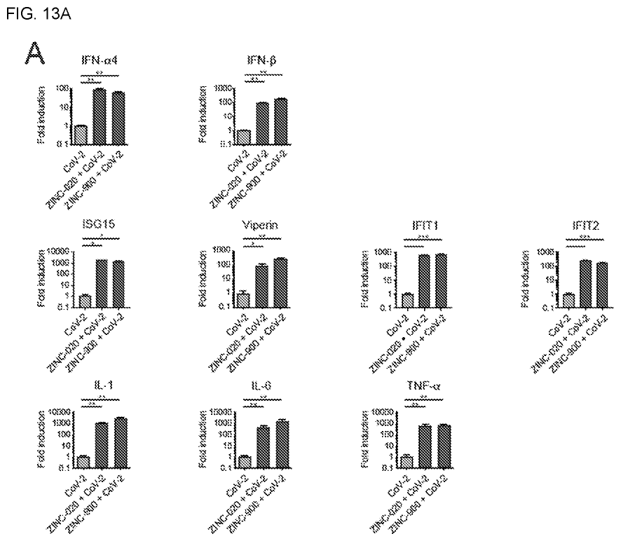

So far, we identified 4 compounds as SARS-CoV-2 inhibitors in end-point experiments that measured cell death after 4 days of viral infection. To further test whether any of the active 4 active compounds in the initial experiment were able to suppress SARS-CoV-2 titers in the cell supernatants during continuous replication over time, we infected Vero cells with SARS-CoV-2 and added 100 μM of the compounds to the culture medium at the time of infection to measure the concentration of infectious virions in the supernatants over the subsequent two days (24 hr and 48 hr). We included ZINC000644163977 again as a negative control. We determined the viral titers in serial dilutions of the supernatants by viral plaque assay and compared them to the viral titers of the positive control cells infected in the absence of the compounds. Consistent with our initial results, ZINC000008077555, ZINC000230464020, ZINC000006623878, and ZINC000085876900 significantly suppressed viral replication by approximately 10-fold on day 2 post infection whereas ZINC000644163977 did not (FIG. 9A-D). Of note, the best individual compound alone achieved the level of inhibition as the combined addition of ZINC000230464020, ZINC000006623878, and ZINC000085876900 to the culture (FIG. 9C-D). We also tested whether select compounds, namely ZINC000230464020, ZINC00000662387, or ZINC000085876900, prevented SARS-CoV-2 replication if they were added 24 hrs prior to infection or 24 hrs after infection. These compounds showed significant suppression of SARS-CoV-2 replication under both conditions, perhaps suggesting a wider therapeutic window (FIG. 10). Our longitudinal results demonstrated that 4 lead compounds ZINC000008077555, ZINC000230464020, ZINC000006623878, and ZINC000085876900 directly inhibited the production of infectious virions 24 hr and 48 hr after infection, as well as significantly inhibited the viral replication when applied 24 hr before or after infection.

Although the tested compounds were selected based on their putative binding to Mpro of SARS-CoV-2 and did not show overt cytotoxicity (FIG. 7A-E), it was possible that unspecific off-target effects on host proteins may also account for the observed reduction of the viral titers in the presence of the compounds, for example through reduced fitness of the host cells. To exclude such possibilities, we tested the ability of any of the 4 active compounds plus one negative control compound to suppress the replication of West Nile virus (WNV), an RNA virus of the unrelated flavivirus family. Infections of Vero cells with WNV in the presence of any of the compounds did not significantly restrict replication of WNV comparing to the untreated controls cells, demonstrating that our 4 active compounds ZINC000008077555, ZINC000230464020, ZINC000006623878, and ZINC000085876900 act specifically on Mpro of SARS-CoV-2 and are not acting on host proteins (FIG. 9E-H). Together, our experiments confirmed compounds ZINC000008077555, ZINC000230464020, ZINC000006623878, and ZINC000085876900 as specific inhibitors of SARS-CoV-2 replication but not interfering with the host cell.

### Mpro Inhibition Promotes Type I IFN and Cytokine Responses.

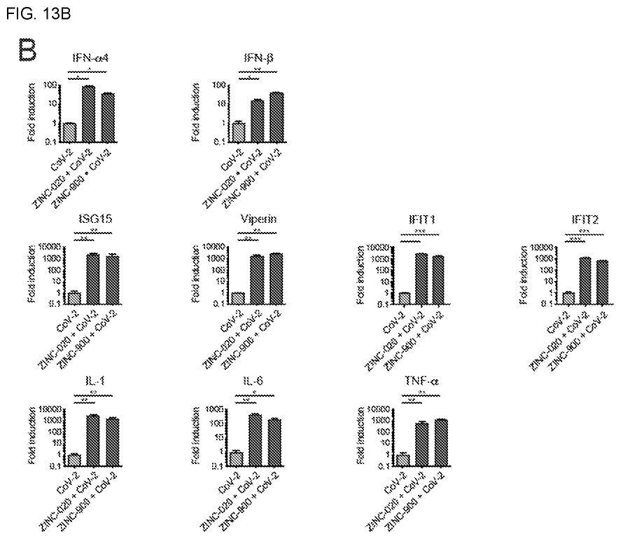

SARS-CoV-2 proteins including Mpro are known to repress the innate immune signaling required for the induction of type I IFNs and proinflammatory cytokines¹⁵. We therefore investigated whether the inhibition of Mpro with the newly identified compounds can enhance the induction of these inflammatory mediators in the infected host cells. To test this, we focused on the two compounds with the highest degree of viral suppression, namely ZINC000230464020 and ZINC000085876900. We infected compound-treated human lung-derived Calu-3 cells with SARS-CoV-2 and measured the expression level of a set of type I IFNs, IFN-sensitive genes (ISGs), and proinflammatory cytokines of these cells 18 hr after infection by RT-qPCR (FIG. 11). Control cells infected in the absence of any compound resulted only in a modest elevation of IFNs, ISGs, and cytokines compared to naïve cells, consistent with blunted IFN and cytokine responses observed in SARS-CoV-2 infections¹⁵ (FIG. 11A-C). Administration of the compounds alone without infection also led to a moderate induction of these genes that at most reached at levels comparable to control cells infected in the absence of any compound. In contrast, infection of Calu-3 cells treated with ZINC000230464020 or ZINC000085876900 resulted in significantly higher expression of these genes compared to the control cells infected in the absence of the compounds. Infection of Vero cells treated with ZINC000230464020 or ZINC000085876900 resulted in similar findings (FIG. 12). Of note, we observed significantly increased expression of these inflammatory genes in compound-treated infected cells despite the reduction of viral RNAs that serve as ligands for the induction of the responses (FIG. 11D, FIG. 12D). In confirmation of this, normalization of the expression level of IFNs, ISGs, and cytokines to the amounts of viral RNAs in the same samples suggested even significantly higher expression levels of these inflammatory mediators genes by our compounds (FIG. 13), highlighting the potency of compound-mediated stimulation of the type I IFN, ISG, and cytokine responses. Together, these data demonstrate that our compounds not only directly inhibit viral replication by binding to Mpro, but also suggest that these compounds released the suppression of cellular anti-viral immune response by the virus. As a consequence, these compounds enable host cells to establish a more robust innate anti-viral state for the suppression of viral replication.

### Activity of SARS-CoV-2 Mpro Inhibitors In Vitro

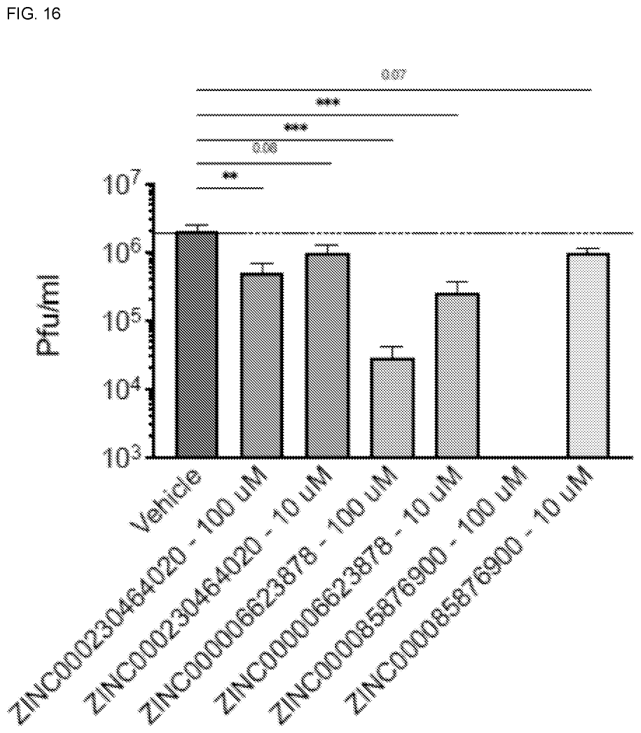

As shown in FIG. 16, Vero cells were infected with SARSCOV-2 (strain WA1, MOI=0.005) in the presence of 10-100 μM of the indicated compounds and 2 μM of the P-gp inhibitor CP-100356. Vehicle-treated cells served as controls. The amounts of infectious virions in the cell supernatants were determined 2 days later by viral plaque assay. ZINC000085876900 was toxic at 100 μM. *, p≤0.05; **, p≤0.005, ***, p<0.0005; Mann-Whitney test.

### Discussion

Mpro, the main protease of SARS-CoV-2, plays a central role in the cleavage of the ORF1-encoded pp1a and pp1ab polypeptides to produce active viral proteins, including the RNA polymerase RdRP. Pharmacological inhibition of Mpro therefore likely inhibits SARS-CoV-2 infection directly by preventing the replication of viral RNA genomes. Across currently 12 VOCs of SARS-CoV-2, the sequence of Mpro is significantly more conserved than the sequence of Spike protein, with only 3 mutations outside its known binding pocket. Therefore, Mpro represent an attractive drug target to interfere with viral replication. Recent studies have reported the possible inhibitors against Mpro of SARS-CoV-2²²⁻²⁶. Indeed, the recent approval of Paxlovid (PF-07321332), a derivative of the Mpro inhibitor GC376, demonstrated this target⁷. While the emergence of the first clinical SARS-CoV-2 Mpro inhibitor is encouraging, it is also clear that the continued expansion of the arsenal of anti-viral drugs is highly desirable.

In experiments conducted during the course of developing embodiments for the invention, we developed an in-silico screening pipeline which integrated the structure-based docking with pharmacokinetics prediction to prioritize approximately 11 million compounds for their ability to bind to Mpro and to block its enzymatic activity in the process of viral replication. To cross-check the binding affinities of the experimentally validated 4 compounds predicted by our in-silico screening pipeline, we examined the binding mode of these compounds; ZINC000230464020 occupied the binding pocket with a Glide docking score of −11.79 kcal/mol, Vina score of −8.73 kcal/mol, and MOE score of −14.81 kcal/mol. It forms seven hydrogen bonds with residues of Gly 143, His164, Glu166, Thr190 and Gln192. ZINC000085876900 located in the binding site with a Glide docking score of −9.00 kcal/mol, Vina score of −9.97 kcal/mol, and MOE score of −7.6 kcal/mol. Four hydrogen bonds were formed between the compound and residues Cys44, Thr190, and Gln192. ZINC000006623878 fitted in the binding pocket with a Glide docking score of −8.99 kcal/mol. Vina score of −7.77 kcal/mol, and MOE score of −6.86 kcal/mol. There are three hydrogen bonds formed between this compound and residue Ser144, Cys145, and Gln189. The hydrogen bond, van der Waals, hydrophobic and Pi-Pi interactions between the validated 4 compounds and SARS-CoV-2 Mpro mainly occurred at the catalytic pocket with strong bonding to His41 and Cys145, indicating these residual may be particularly responsible for inhibiting SARS-CoV-2 Mpro (FIG. 6). In addition, none of the 9 tested compounds were predicted to be toxic, which is consistent with our in vitro findings.

To test their inhibitory activity of SARS-CoV-2, we performed two rounds of in vitro experiments. The initial in vitro infection of cells with SARS-CoV-2 identified 4 out of the 9 lead compounds (ZINC000008077555, ZINC000230464020, ZINC000085876900, and ZINC000006623878) as potentially inhibitors of SARS-CoV-2 infection. We further fully confirmed the inhibition of SARS-CoV-2 infection for these compounds using a diverse set of assays in two distinct cell lines, namely African green monkey Vero cells and human lung Calu-3 cells. These compounds showed no overt cytotoxicity. The fact that they inhibited SARS-CoV-2 but not WNV indicates that our compounds are highly specific for SARS-CoV-2 and do not affect unrelated RNA viruses or host proteins.

Aside from its function in the processing of the proteins directly required for viral replication, such as the RNA polymerase. Mpro is also important for the proteolytic cleavage of other ORF1-encoded non-structural proteins that modulate the physiology of the host cells. Among the functions of those proteins, including Mpro itself, is the inhibition of numerous cellular signaling molecules necessary for the induction of anti-viral innate immune responses¹³⁻¹⁵. Inhibition of Mpro may thus promote anti-viral immunity of the host cells by interfering with this suppression mechanism, thus facilitating the sensing of the viral RNA by the innate immune system. Our observation that ZINC000008077555, ZINC000230464020, ZINC000085876900, and ZINC000006623878 all enhance type I IFNs and proinflammatory cytokines as well as ISGs that are central for the anti-viral state in infected cells is consistent with such an additional mechanism of action. Beyond the immediate cellular defense by creating an anti-viral state, it is likely that the increased type I IFNs and proinflammatory cytokine responses are particularly beneficial in vivo. Here, the restoration of these innate immune responses of infected cells by the inhibition of Mpro presumably enhances the recruitment and function of other innate immune cells, thus amplifying the anti-viral effect of the drug candidates^{15,27,28}. In addition, enhanced type I and type III responses may also be beneficial in vivo as dysregulated and delayed IFN responses are a hallmark of COVID-19 patients with severe disease¹⁵. As the early administration of type I IFNs has been proposed as treatment to counter this effect, it is possible that Mpro inhibition has a positive impact on disease severity that is independent of its direct or indirect role in limiting viral load. Future experiments will address such possibilities.

### Example II

This example described the materials and methods utilized in conducting the experiments recited in Example I.

### Sequence Alignment of Spike and 3C-Like Proteinase and its Variants

The protein sequences of Spike glycoprotein variants and 3C-like proteinase variants of SARS-CoV-2 isolates were retrieved from NCBI https://www.ncbi.nlm.nih.gov/datasets/coronavirus/proteins/). The Spike protein, 3C-like proteinase and its variants sequences were extracted using our UNIX script. To determine the level of the conservancy, multiple sequence alignment (MSA) was performed for the sequences using the BioEdit-ClustalW multiple alignment program (http://www.mbio.ncsu edu/BioEdit/bioedit.html). After multiple alignment for all the download sequences for each variant, we created a consensus sequence. Next, we used Clustal Omega (https://www.ebi.ac.uk/Tools/msa/clustalo/) to align consensus sequence across all the VOCs of SARS-CoV-2, SARS-COV, and MERS-COV.

### Virtual Screening

To identify the potential small molecular inhibitors for SARDS-CoV-2 main protease (Mpro), the structure-based virtual screening was carried out by Virtual Screening Workflow (VSW) from Schrödinger suites²⁰. A total around 11 million Drug-Like In-Stock 3D small molecules obtained from ZINC database¹⁹ were performed through VSW. Mpro structure was prepared using Protein Preparation Wizard in Maestro from Schrödinger²⁹. Hydrogens were added to the protein and bond orders were assigned. All hydrogen-bonding networks were optimized, and the ionization states were assigned at pH 7.0. OPLS3e force field was used for restrained minimization. The docking grid was generated with the Receptor Grid Generation tool from Maestro. The default van der Waals scaling factor of 1.0 and partial charge cutoff at 0.25 was applied³⁰. The center and the size of the grid box were defined according to the position of the published inhibitor X77 in the crystal structure of Mpro (PDB ID: 6W63). To dock ligands with suitable pharmacological property, the small molecules were prefiltered by removing ligands with reactive functional groups. Around 10.4 million compounds were obtained. Virtual screening was carried out in three sequential steps, namely (a) Glide high throughput virtual screening (HTVS) docking; After HTVS docking, (b) Glide standard precision (SP) docking, and finally (c) Glide extra precision (XP) docking. At each step, the top 10% of the compounds were advanced to the next step. Finally, according to the Glide score and protein-ligand interactions, top 500 lead compounds were selected for further evaluations.

### Vina and MOE Docking.

Different docking methods, including Vina docking and MOE docking, were used to predict the binding affinity of the top 500 lead compounds which were prioritized based on previous Glide score against Mpro. Auto Dock Vina is a widely used open-source docking program³¹. The Mpro and structures of the top 500 lead compounds were prepared with Auto Dock Tools³². All hydrogens were added and Gasteiger charges were assigned. The center and the size of the grid box were defined according to the position of the published inhibitor X77 (https://www.rcsb.org/structure/6W63). The level of exhaustiveness were set to 8. The Auto Dock Vina docking score was used to calculate the free energy of binding of different docking poses. The docking calculations were repeated 3 times with different random seeds.

Next, we employed MOE docking^{33,34} to calculate the binding affinity of the top 500 lead compounds. The protein was kept as rigid, and a maximum of 30 conformations for each ligand was tested, using the default parameters of MOE using Triangle Matcher placement. The top ranked conformations of lead molecules were stored. On the basis of MOE scoring (London dG), binding free energy calculation in the S field was scored, as the London dG is a scoring function that estimates the free energy of binding of the ligand for a given pose. For all scoring functions, lower scores indicate more favorable poses.

### Toxicity Prediction

The in-silico toxicity properties were predicted by Data Warrior³⁵. Data Warrior was used to predict the molecular weight (MW), mutagenicity, tumorigenicity, and irritant properties as well as pharmacokinetic properties, Topological Molecular Surface area (TPSA), partition coefficient (log P) for the identified top 500 molecules. Toxicity risks were predicted from precompiled lists of fragments using an algorithm that gives rise to toxicity alerts in case they encounter the structure in evaluation 36.

### Top 500 Lead Compounds Prioritization

To prioritize robust compounds for experiment validation, we developed an ensemble of two complementary strategies. In the first strategy, we employed a screening-driven approach to prioritize robust lead compounds. We first ranked the top 500 lead compounds based on the average (priority score) of the individual Glide, Vina and MOE docking scores. Next, we selected top 50 lead compounds with the highest priority score and performed binding mode clustering by Schrödinger and structure similarity clustering by Data Warrior. Among the top 50 compounds, we identified 10 binding mode clusters and 23 structure similarity clusters (Table 5). We removed 6 compounds based on the predicted toxic pharmacological properties by Data Warrior (Table 5). For the remaining 44 compounds, we first selected the top compound with the highest priority score within each binding mode cluster to obtain total 9 compounds (Table 5). Next, we examined the 9 compounds against their structure similarity and selected the top compound per structure similarity cluster with highest priority score. Note that 3 of the 9 compounds belong to the same similarity cluster, so the final number of selected compounds is 7 (Table 5). Also, 4 out of the final selected 7 compounds are commercially available and are used for the subsequent in-vitro infection experiments.

TABLE 5
Prioritization of lead inhibitors of SARS-CoV-2 Mpro based on screening-driven approach.

| ZINC_ID (see FIG. 14 | Glide | | | | Average | MOE |
|---|---|---|---|---|---|---|
| for corresponding | docking | Vina | Vina | Vina | Vina | docking |
| structure) | score | docking 1 | docking 2 | docking 3 | docking | score |
| ZINC000085591448 | −10.524705 | −7.3 | −8.3 | −7.7 | −7.7666667 | −13.0422 |
| ZINC000230464020 | −11.79488 | −8.8 | −8.7 | −8.7 | −8.7333333 | −16.7152 |
| ZINC000104216484 | −11.019224 | −9.5 | −9.6 | −9.3 | −9.4666667 | −15.9223 |
| ZINC000217772611 | −11.79488 | −8.3 | −8.8 | −8.7 | −8.6 | −16.7152 |
| ZINC000068056786 | −11.039482 | −8.7 | −9.1 | −9 | −8.9333333 | −14.8821 |
| ZINC000217605126 | −11.267892 | −7.9 | −8.1 | −8 | −8 | −14.5738 |
| ZINC000021659122 | −10.513891 | −10.5 | −10.5 | −10.5 | −10.5 | −14.8996 |
| ZINC000071766838 | −10.760459 | −8.5 | −8.1 | −8.4 | −8.3333333 | −14.4788 |
| ZINC000070707665 | −10.713601 | −8.5 | −8.6 | −8.7 | −8.6 | −13.7636 |
| ZINC000097950308 | −10.915662 | −8.3 | −8.7 | −8.8 | −8.6 | −12.8305 |
| ZINC000256678124 | −10.774729 | −7.4 | −7.5 | −7.9 | −7.6 | −14.5216 |
| ZINC000238784810 | −10.559997 | −9.2 | −9.3 | −9.2 | −9.2333333 | −13.5214 |
| ZINC000005273910 | −10.593462 | −8.6 | −8.6 | −8.6 | −8.6 | −13.6399 |
| ZINC000253487193 | −10.531139 | −9.2 | −9.2 | −9 | −9.1333333 | −12.9521 |
| ZINC000012243534 | −10.585587 | −7.7 | −7.8 | −8 | −7.8333333 | −13.6763 |
| ZINC000252428459 | −10.623038 | −8.9 | −8.2 | −8.3 | −8.4666667 | −12.8435 |
| ZINC000217685606 | −10.160838 | −9.2 | −9.2 | −8.9 | −9.1 | −13.7209 |
| ZINC000021659003 | −10.20991 | −11.1 | −11.1 | −11.1 | −11.1 | −13.356 |
| ZINC000150445510 | −10.598844 | −9.6 | −8.8 | −9.7 | −9.3666667 | −11.9554 |
| ZINC000008214413 | −11.503407 | −6.5 | −6.4 | −6.4 | −6.4333333 | −13.6554 |
| ZINC000150340729 | −12.105904 | −6.3 | −5.7 | −5.9 | −5.9666667 | −13.3933 |
| ZINC000253504166 | −10.157409 | −8.1 | −8.5 | −7.8 | −8.1333333 | −14.7404 |
| ZINC000150381094 | −10.779623 | −8.1 | −7.2 | −7.1 | −7.4666667 | −12.8222 |
| ZINC000644163976 | −11.709913 | −7.7 | −7.8 | −7.1 | −7.5333333 | −11.7724 |
| ZINC000257355070 | −10.961052 | −6.7 | −6.9 | −7.2 | −6.9333333 | −13.3803 |
| ZINC000027301133 | −10.139927 | −8.3 | −8 | −8.5 | −8.2666667 | −13.6281 |
| ZINC000644163932 | −10.437952 | −7.3 | −7.1 | −7 | −7.1333333 | −14.1605 |
| ZINC000644163980 | −10.411884 | −7.7 | −8 | −7.7 | −7.8 | −13.2838 |
| ZINC000085540223 | −10.924991 | −7.1 | −7.2 | −6.8 | −7.0333333 | −12.7341 |
| ZINC000005462364 | −10.669735 | −6.7 | −6.6 | −6.7 | −6.6666667 | −13.4752 |
| ZINC000082185317 | −10.363284 | −8.3 | −8.3 | −8.3 | −8.3 | −12.6244 |
| ZINC000005462424 | −10.223268 | −6.9 | −7 | −7.1 | −7 | −15.0554 |
| ZINC000644163939 | −10.5853 | −8.5 | −8.1 | −8.1 | −8.2333333 | −11.5591 |
| ZINC000239025333 | −10.184102 | −8.2 | −8.1 | −8.3 | −8.2 | −12.7907 |
| ZINC000238899980 | −10.306421 | −7 | −7.1 | −7 | −7.0333333 | −12.4929 |
| ZINC000030836149 | −10.12688 | −7.4 | −7.3 | −7.4 | −7.3666667 | −12.3159 |
| ZINC000644163974 | −10.738047 | −7.7 | −7.4 | −7.4 | −7.5 | −11.4608 |
| ZINC000644164296 | −10.607948 | −7.2 | −7.1 | −7.6 | −7.3 | −11.2554 |
| ZINC000097972782 | −11.000477 | −6.9 | −6.6 | −6.9 | −6.8 | −12.579 |
| ZINC000255985724 | −10.213452 | −7.2 | −7.6 | −7.3 | −7.3666667 | −12.7046 |
| ZINC000607715769 | −10.222577 | −7.7 | −8.6 | −7.2 | −7.8333333 | −13.7404 |
| ZINC000644163966 | −10.253545 | −7.3 | −7.5 | −7.2 | −7.3333333 | −11.3554 |
| ZINC000255974857 | −10.645495 | −6.9 | −7.8 | −7.7 | −7.4666667 | −13.6621 |
| ZINC000255974854 | −10.238238 | −7.5 | −7.3 | −7.1 | −7.3 | −15.2163 |
| ZINC000644163891 | −10.751981 | −6.7 | −6.3 | −6.4 | −6.4666667 | −12.6426 |
| ZINC000150484663 | −10.121432 | −7.9 | −8 | −7.4 | −7.7666667 | −12.449 |
| ZINC000328577326 | −10.120995 | −7.7 | −7.4 | −7.8 | −7.6333333 | −12.1024 |
| ZINC000100072156 | −10.67729 | −7.9 | −7.8 | −8.5 | −8.0666667 | −15.4678 |
| ZINC000150397896 | −10.501281 | −7.5 | −7.8 | −6.7 | −7.3333333 | −15.3273 |
| ZINC000644163977 | −10.315868 | −8.9 | −7.8 | −8.9 | −8.5333333 | −13.2668 |

| ZINC_ID (see FIG. 14 | | | | | | Binding | |
|---|---|---|---|---|---|---|---|
| for corresponding | Glide | Vina | MOE | Average | Similarity | mode | |
| structure) | rank | rank | rank | rank | cluster | cluster | Molweight |
| ZINC000085591448 | 30 | 27 | 30 | 29 | 8 | 1 | 753.387 |
| ZINC000230464020 | 2 | 9 | 1 | 4 | 2 | 2 | 533.535 |
| ZINC000104216484 | 8 | 3 | 3 | 4.66666667 | 5 | 2 | 629.661 |
| ZINC000217772611 | 3 | 10 | 2 | 5 | 2 | 2 | 533.535 |
| ZINC000068056786 | 7 | 8 | 9 | 8 | 4 | 2 | 688.74 |
| ZINC000217605126 | 6 | 23 | 11 | 13.3333333 | 2 | 2 | 548.55 |
| ZINC000021659122 | 31 | 2 | 8 | 13.6666667 | 18 | 2 | 500.941 |
| ZINC000071766838 | 15 | 16 | 13 | 14.6666667 | 3 | 2 | 522.576 |
| ZINC000070707665 | 18 | 12 | 15 | 15 | 4 | 2 | 551.534 |
| ZINC000097950308 | 12 | 11 | 33 | 18.6666667 | 6 | 2 | 518.54 |
| ZINC000256678124 | 14 | 30 | 12 | 18.6666667 | 8 | 2 | 667.441 |
| ZINC000238784810 | 28 | 5 | 23 | 18.6666667 | 16 | 2 | 657.555 |
| ZINC000005273910 | 25 | 13 | 21 | 19.6666667 | 3 | 2 | 507.541 |
| ZINC000253487193 | 29 | 6 | 31 | 22 | 17 | 2 | 713.754 |
| ZINC000012243534 | 26 | 24 | 18 | 22.6666667 | 14 | 2 | 490.986 |
| ZINC000252428459 | 22 | 15 | 32 | 23 | 12 | 2 | 601.638 |
| ZINC000217685606 | 45 | 7 | 17 | 23 | 22 | 2 | 541.539 |
| ZINC000021659003 | 43 | 1 | 27 | 23.6666667 | 18 | 2 | 480.523 |
| ZINC000150445510 | 24 | 4 | 45 | 24.3333333 | 13 | 2 | 676.796 |
| ZINC000008214413 | 5 | 49 | 20 | 24.6666667 | 1 | 2 | 777.075 |
| ZINC000150340729 | 1 | 50 | 25 | 25.3333333 | 1 | 2 | 835.154 |
| ZINC000253504166 | 46 | 21 | 10 | 25.6666667 | 23 | 2 | 670.582 |
| ZINC000150381094 | 13 | 33 | 34 | 26.6666667 | 7 | 2 | 680.709 |
| ZINC000644163976 | 4 | 31 | 46 | 27 | 3 | 2 | 699.779 |
| ZINC000257355070 | 10 | 45 | 26 | 27 | 3 | 2 | 784.933 |
| ZINC000027301133 | 47 | 18 | 22 | 29 | 15 | 2 | 572.684 |
| ZINC000644163932 | 33 | 41 | 14 | 29.3333333 | 3 | 2 | 594.667 |
| ZINC000644163980 | 34 | 26 | 28 | 29.3333333 | 15 | 2 | 675.76 |
| ZINC000085540223 | 11 | 42 | 36 | 29.6666667 | 1 | 2 | 791.102 |
| ZINC000005462364 | 20 | 47 | 24 | 30.3333333 | 3 | 2 | 411.477 |
| ZINC000082185317 | 35 | 17 | 39 | 30.3333333 | 5 | 2 | 728.654 |
| ZINC000005462424 | 40 | 44 | 7 | 30.3333333 | 3 | 2 | 498.555 |
| ZINC000644163939 | 27 | 19 | 47 | 31 | 15 | 2 | 677.776 |
| ZINC000239025333 | 44 | 20 | 35 | 33 | 3 | 2 | 562.621 |
| ZINC000238899980 | 37 | 43 | 41 | 40.3333333 | 19 | 2 | 410.421 |
| ZINC000030836149 | 48 | 36 | 43 | 42.3333333 | 3 | 2 | 442.447 |
| ZINC000644163974 | 17 | 32 | 48 | 32.3333333 | 3 | 3 | 699.779 |
| ZINC000644164296 | 23 | 39 | 50 | 37.3333333 | 3 | 3 | 782.893 |
| ZINC000097972782 | 9 | 46 | 40 | 31.6666667 | 3 | 4 | 784.933 |
| ZINC000255985724 | 42 | 35 | 37 | 38 | 11 | 4 | 620.709 |
| ZINC000607715769 | 41 | 25 | 16 | 27.3333333 | 21 | 5 | 794.942 |
| ZINC000644163966 | 38 | 38 | 49 | 41.6666667 | 20 | 6 | 772.882 |
| ZINC000255974857 | 21 | 34 | 19 | 24.6666667 | 11 | 7 | 688.78 |
| ZINC000255974854 | 39 | 40 | 6 | 28.3333333 | 11 | 7 | 688.78 |
| ZINC000644163891 | 16 | 48 | 38 | 34 | 9 | 7 | 674.794 |
| ZINC000150484663 | 49 | 28 | 42 | 39.6666667 | 15 | 7 | 781.929 |
| ZINC000328577326 | 50 | 29 | 44 | 41 | 3 | 8 | 779.869 |
| ZINC000100072156 | 19 | 22 | 4 | 15 | 10 | 9 | 616.535 |
| ZINC000150397896 | 32 | 37 | 5 | 24.6666667 | 5 | 10 | 757.835 |
| ZINC000644163977 | 36 | 14 | 29 | 26.3333333 | 15 | 10 | 675.76 |

| ZINC_ID (see FIG. 14 | | | | LE from | | | |
|---|---|---|---|---|---|---|---|
| for corresponding | | | | Total | | | |
| structure) | cLogP | cLogS | Druglikeness | Molweight | Mutagenic | Tumorigenic | Irritant |
| ZINC000085591448 | −13.926 | −1.296 | −38.717 | 0.17143 | none | none | none |
| ZINC000230464020 | 2.0744 | −4.817 | −1.7465 | 0.22066 | none | none | none |
| ZINC000104216484 | −6.6747 | −4.998 | 4.8395 | 0.18904 | none | none | high |
| ZINC000217772611 | 2.0744 | −4.817 | −1.7465 | 0.22066 | none | none | none |
| ZINC000068056786 | −1.7667 | −3.69 | 9.393 | 0.16907 | none | none | none |
| ZINC000217605126 | 0.7578 | −4.504 | 5.7054 | 0.21473 | none | none | none |
| ZINC000021659122 | 4.1051 | −7.933 | 2.6209 | 0.24009 | none | none | none |
| ZINC000071766838 | −1.8326 | −3.341 | 0.49493 | 0.22679 | none | none | none |
| ZINC000070707665 | −3.4789 | −3.631 | 5.3132 | 0.21465 | none | none | none |
| ZINC000097950308 | 0.1316 | −5.23 | −0.1868 | 0.22691 | none | high | none |
| ZINC000256678124 | −10.584 | −0.348 | −35.433 | 0.19703 | none | none | none |
| ZINC000238784810 | −1.7699 | −4.525 | 0.44974 | 0.18045 | none | none | none |
| ZINC000005273910 | −0.693 | −3.213 | 3.1081 | 0.23339 | none | none | none |
| ZINC000253487193 | 0.6787 | −5.059 | −17.614 | 0.16534 | none | none | none |
| ZINC000012243534 | 2.6188 | −6.55 | 3.9655 | 0.26228 | none | none | none |
| ZINC000252428459 | −1.7488 | −4.882 | −5.5251 | 0.19395 | high | high | none |
| ZINC000217685606 | −0.767 | −5.114 | 5.146 | 0.21492 | none | none | none |
| ZINC000021659003 | 3.843 | −7.541 | 2.53 | 0.24078 | none | none | none |
| ZINC000150445510 | 4.6985 | −6.265 | 9.7217 | 0.17273 | none | none | none |
| ZINC000008214413 | −1.3007 | −3.813 | 5.4021 | 0.27037 | none | none | none |
| ZINC000150340729 | −1.0339 | −4.112 | 5.9269 | 0.23825 | none | none | none |
| ZINC000253504166 | −3.5451 | −3.165 | 2.9425 | 0.17645 | none | none | none |
| ZINC000150381094 | −3.3961 | −1.976 | −12.504 | 0.17626 | none | none | none |
| ZINC000644163976 | −5.681 | −3.169 | 0.82723 | 0.16888 | none | none | none |
| ZINC000257355070 | −2.6443 | −4.541 | 2.873 | 0.14956 | none | none | none |
| ZINC000027301133 | −1.9073 | −4.05 | 6.7615 | 0.20389 | none | none | none |
| ZINC000644163932 | −7.4368 | −2.35 | 2.8702 | 0.20336 | none | none | none |
| ZINC000644163980 | −3.6621 | −3.5 | 6.527 | 0.17275 | none | none | none |
| ZINC000085540223 | −0.8944 | −4.113 | 5.5768 | 0.26159 | none | none | none |
| ZINC000005462364 | −2.9414 | −1.785 | −0.47956 | 0.30208 | none | none | none |
| ZINC000082185317 | 1.6514 | −5.672 | 2.9146 | 0.16192 | none | none | high |
| ZINC000005462424 | −4.4251 | −1.423 | 0.39821 | 0.24703 | none | none | none |
| ZINC000644163939 | −1.7523 | −3.999 | 3.8791 | 0.17271 | none | none | none |
| ZINC000239025333 | 1.2241 | −4.453 | −10.181 | 0.20912 | none | none | none |
| ZINC000238899980 | −0.2939 | −3.513 | 0.705 | 0.30213 | none | none | none |
| ZINC000030836149 | −1.5369 | −3.317 | −9.9357 | 0.27241 | none | none | none |
| ZINC000644163974 | −5.681 | −3.169 | 0.82723 | 0.16888 | none | none | none |
| ZINC000644164296 | −3.4507 | −3.823 | 3.7927 | 0.14959 | none | none | none |
| ZINC000097972782 | −2.6443 | −4.541 | 2.873 | 0.14956 | none | none | none |
| ZINC000255985724 | −7.5203 | −1.836 | 3.7267 | 0.19353 | none | none | none |
| ZINC000607715769 | −0.5261 | −5.488 | 7.237 | 0.14681 | none | none | none |
| ZINC000644163966 | −4.6797 | −4.055 | −2.3631 | 0.15527 | none | none | none |
| ZINC000255974857 | −5.639 | −3.561 | 1.0493 | 0.17252 | none | none | none |
| ZINC000255974854 | −5.639 | −3.561 | 1.0493 | 0.17252 | none | none | none |
| ZINC000644163891 | −7.8562 | −1.663 | −2.1197 | 0.18012 | none | none | none |
| ZINC000150484663 | −3.9254 | −3.806 | 6.9353 | 0.1496 | none | none | none |
| ZINC000328577326 | −7.1248 | −3.823 | 2.674 | 0.14963 | none | none | none |
| ZINC000100072156 | −8.3558 | −0.255 | 0.5526 | 0.19813 | low | none | none |
| ZINC000150397896 | −7.193 | −5.522 | −0.60693 | 0.15549 | none | none | high |
| ZINC000644163977 | −3.6621 | −3.5 | 6.527 | 0.17275 | none | none | none |

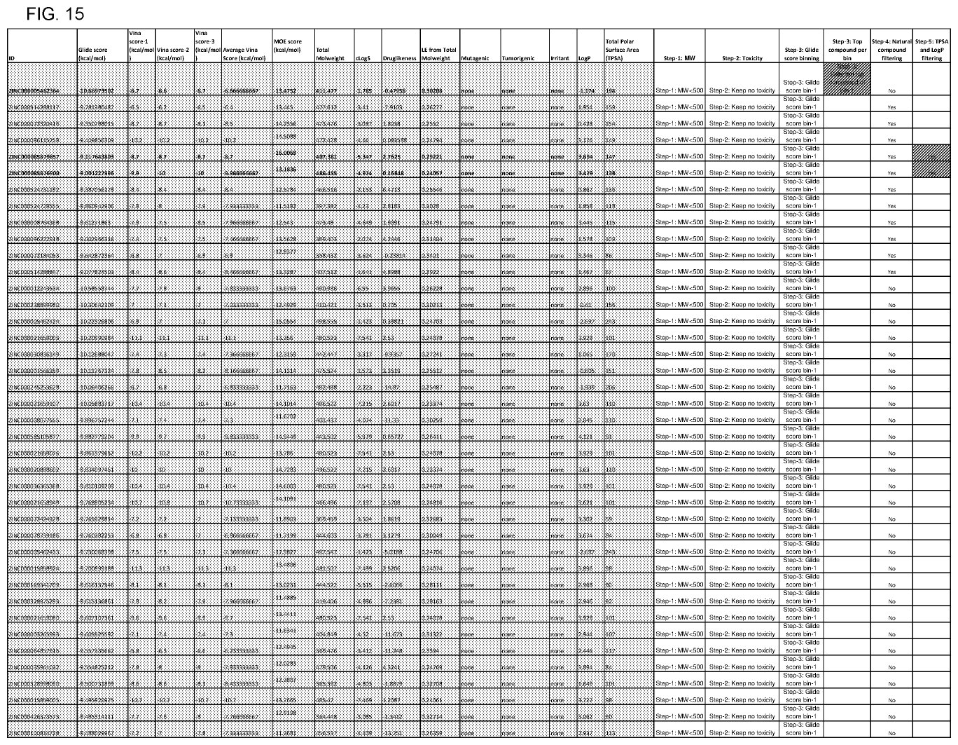

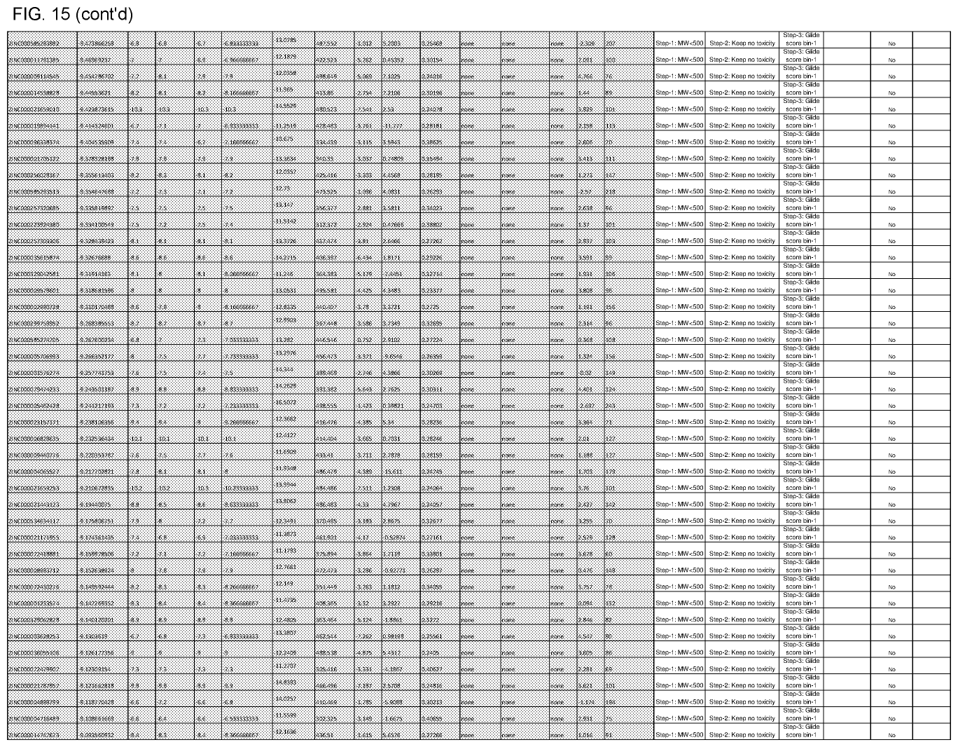

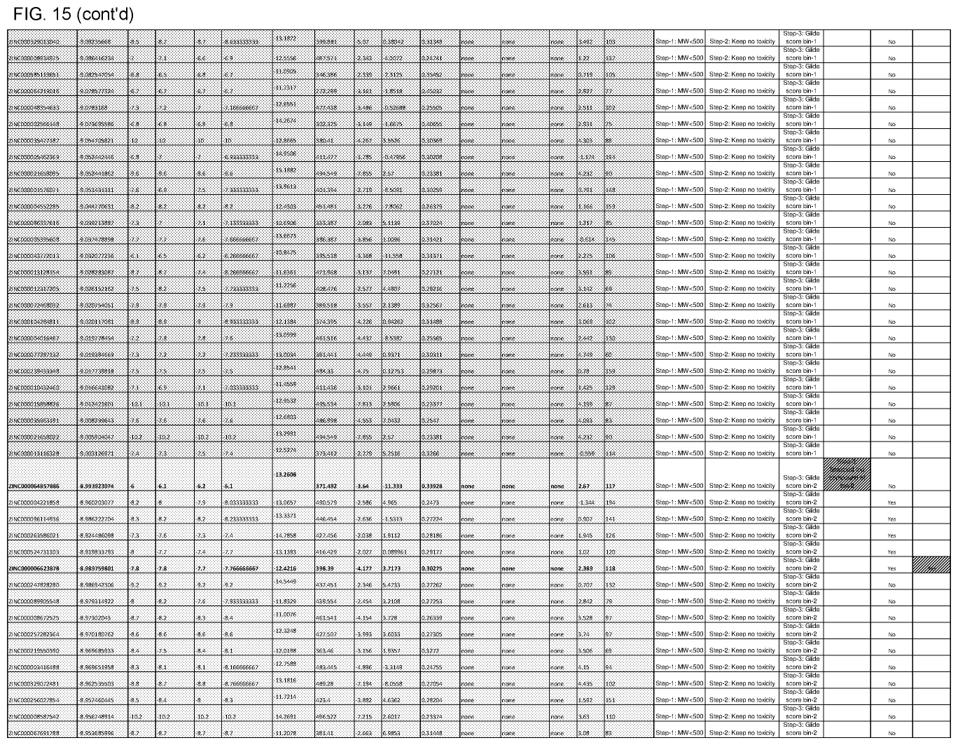

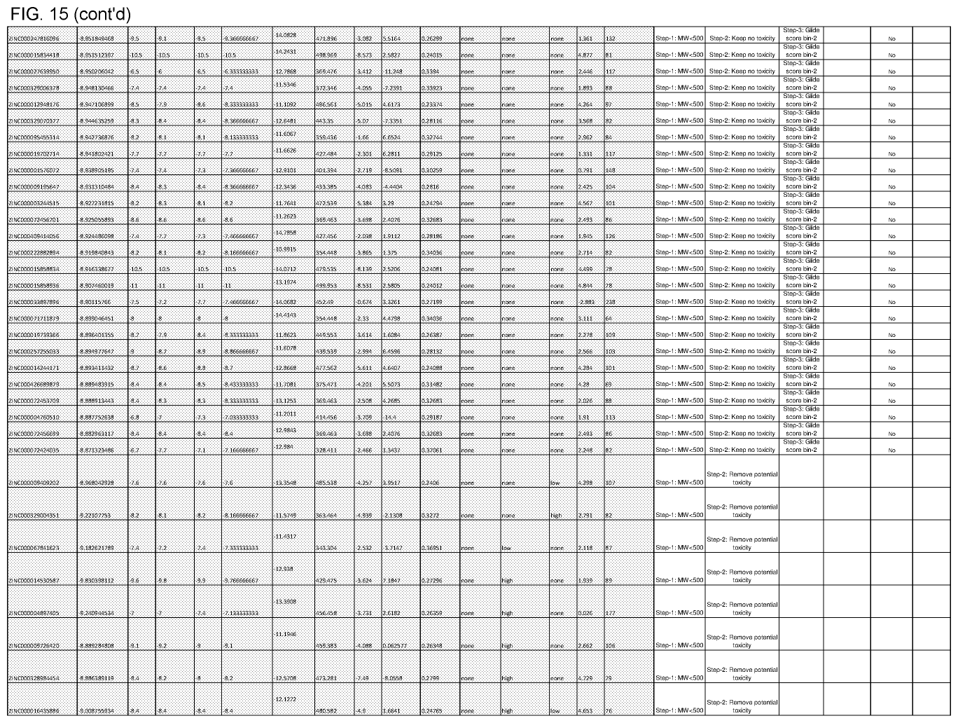

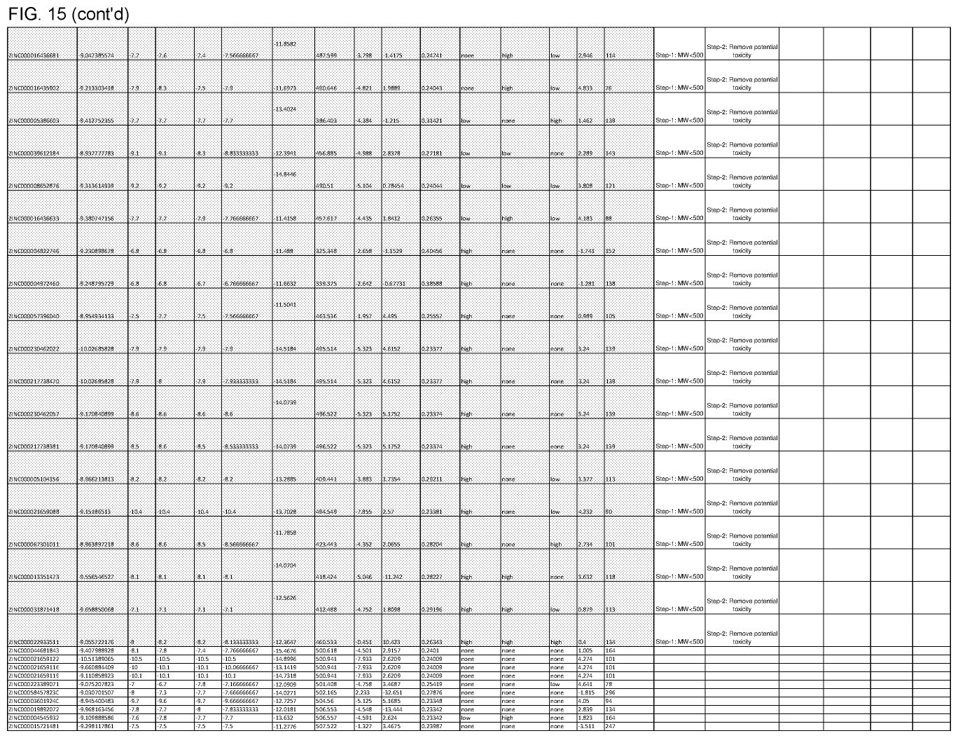

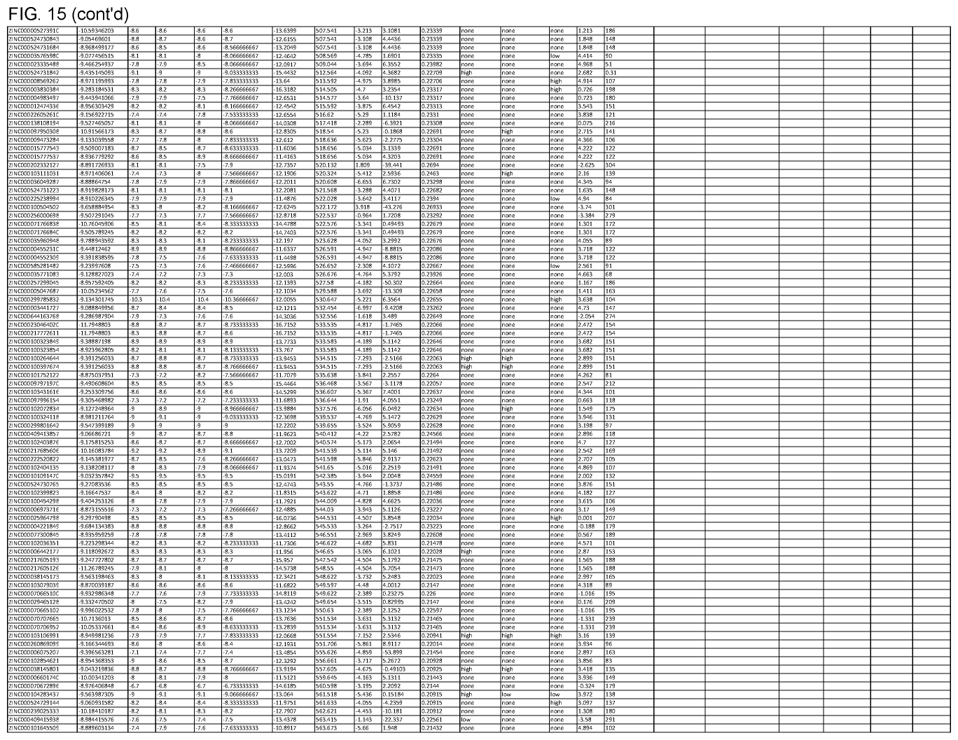

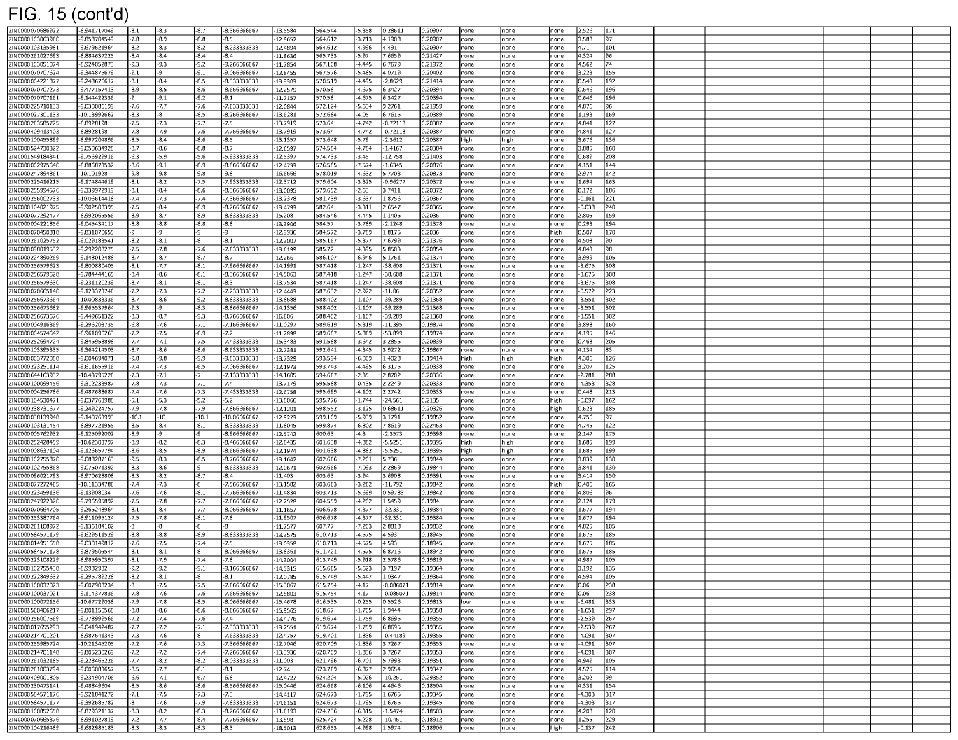

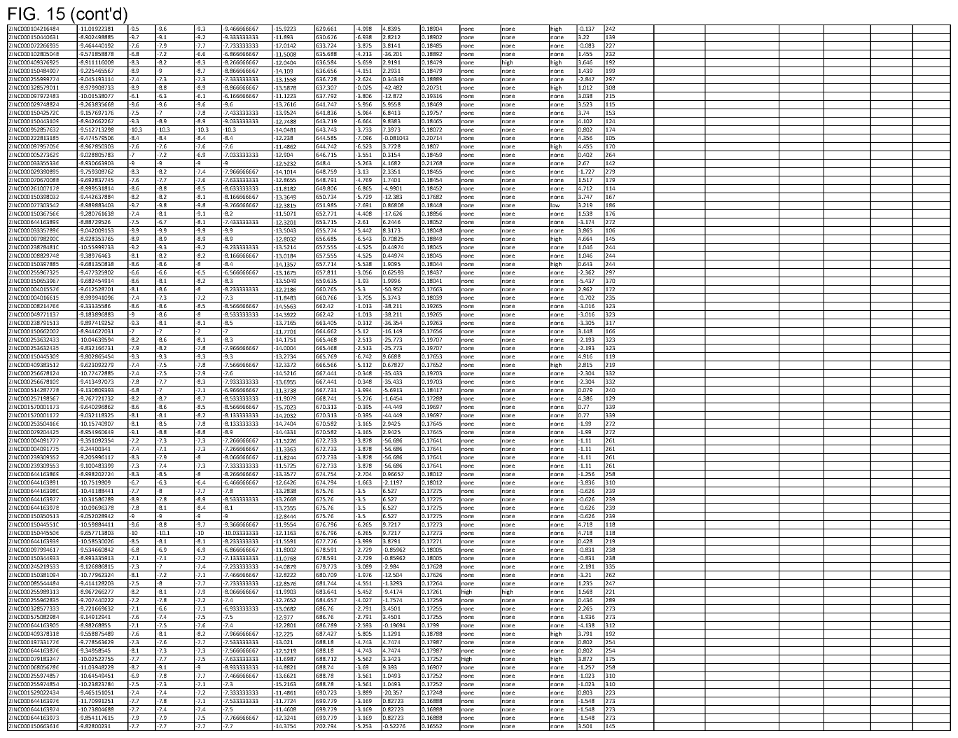

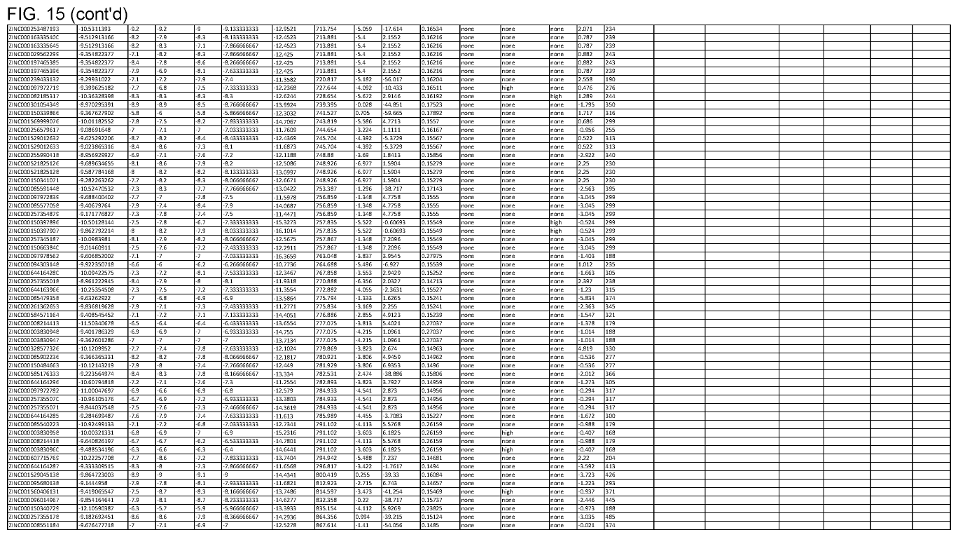

In the second strategy, we employed a pharmacology-informed approach to prioritize the top 500 compounds by integrating the docking score (Glide score) with molecular weight (MW) mutagenicity, tumorigenicity, and irritant properties as well as pharmacokinetic properties, the total polar surface area (TPSA)³⁷ and the partition coefficient (Log P)³⁸ of these compounds (Table 6). First, we removed 322 compounds with molecular weight greater than 500 Daltons resulting in 178 compounds (Table 6). Second, we used the same toxicity criteria as above to remove 27 potentially toxic compounds. Third, we ranked the remaining 151 compounds according to their Glide score which ranges from −10.67 to −8.87 and we divided the scores into two bins: [−10.67, −9] and [−9,−8]. We selected the top compound from each bin (ZINC000005462364, ZINC000064857886). Fourth, since natural compounds are more accessible, can have anti-viral effects against SARS-CoV-2³⁹⁻⁴⁴, and may help to accelerate drug development, we focused on prioritizing natural compounds for the remaining 149 compounds, of which 16 are natural compounds according to ZINC database classification¹⁹. Next, since drug-like molecules should be water-soluble to reach target tissues and enter cells through passive mechanisms such as the diffusion through cellular membranes. The ideal distribution coefficient for the tested compounds should therefore be neither too lipophilic nor too hydrophilic. Such pharmacological properties determines the good absorption and distribution in vivo and guide the translation of chemical inhibitors or viral replication into successful drugs for patients⁴⁵. To select drug-like natural compounds, we set TPSA values between 118 and 148 and Log P values between 2 and 4 following previous published practice^{37,38}, which resulted in 3 out of the 16 natural compounds. In total, we prioritized 5 compounds (FIG. 15) by pharmacology-informed approach which were all commercially available for experimental testing.

In summary, we selected 7 compounds by screening-driven approach, out of which 4 compounds are commercially available, and 5 compounds by pharmacology-informed approach, all of which are commercially available. Therefore, total 9 compounds were ordered for experimental validations.

### Cells.

Vero and Calu-3 cells were obtained from the American Type Culture Collection (ATCC) and cultured according to the recommendations provided by the ATCC. The cells were routinely monitored for the absence of Mycoplasma infection.

### Compounds.

Potential inhibitors of Mpro identified in the virtual screens were obtained from MolPort and eMolecules and dissolved at 10 mM in PBS or PBS+10% DMSO. The compounds were further diluted >100-fold in tissue culture medium containing 5% FCS to obtain working concentrations for the viral replication assays.

### Virus Production.

SARS-CoV-2 strain WA1 and West Nile virus (WNV) strain NY99 were obtained from BEI Resources and propagated in Vero cells. The cells were infected with SARS-CoV-2 at an MOI of 0.005 and with WNV at an MOI of 0.01. After 48 hrs (SARS-CoV-2) or 72 hrs (WNV) of culture, the cells were harvested with a cell scraper and spun and together with the culture medium at 3000 rpm for 10 min. Supernatants were set aside while the resuspended cell pellets were treated with a Dounce homogenizer and subjected to two freeze-thaw cycles before combined with the original supernatants. Following an additional centrifugation step, supernatants were aliquoted, frozen, and subsequently titered in serial dilutions by viral plaque assay. All work with SARS-CoV-2 and WNV was performed under BSL3 conditions in a facility with negative pressure and PPE that included Tyvek suits and N95 masks for respiratory protection.

### Viral Plaque and Foci Assays.

The number of infectious SARS-CoV-2 virions was quantified by viral plaque assay. To this end, Vero cells were incubated with SARS-CoV-2 for 2 hrs and subsequently overlaid with 1% methylcellulose in culture medium. After 3-4 days, the cells were fixed in 10% formalin for 30 min, washed under tap water, and stained with crystal violet. The number of plaques corresponding to infections of individual cells by single virions was counted on a light table. The quantification of infectious WNV virions was performed similarly with the exception that the number of infected cell foci was determined by intracellular staining using a biotinylated anti-WNV-E antibody (clone E16), followed by an HRP-labeled anti-streptavidin antibody. HRP activity was detected with KPL Trueblue substrate (SeraCare).

### Cytopathic Effect and Cytotoxicity Measurements and Other Assays to Determine Drug Activity.

Viral replication of SARS-CoV-2 in the presence of Mpro inhibitors was measured in Vero or Calu-3 cells in three ways. In the first approach, Vero or Calu-3 cells were infected with SARS-CoV-2 at an MOI of 0.01 in the presence of indicated concentrations of Mpro inhibitors and, if applicable, with 1.5 μM of the P-gp inhibitor CP-100356. The virus-induced cytopathic effect was measured by determining the fraction of formalin-fixed adherent cells that remained after 3 days (Vero cells) or 4 days (Calu-3 cells). To this end, the cells were stained with crystal violet, PBS-washed, and air-dried. Following resuspension in methanol, crystal violet staining was measured in the spectrophometer at OD₅₉₄. Cytotoxicity of the compounds was measured in parallel by the staining of uninfected cells incubated with the Mpro. In a second approach, drug activity was determined in Vero cells directly by viral plaque assay, using between 100-500 Pfu/well of virus and indicated concentrations of Mpro inhibitors. In a third approach, Vero cells were infected with SARS-CoV-2 or WNV at an MOI of 0.01 in the presence of 100 mM of Mpro inhibitors for 2-3 days. Viral replication was measured indirectly at indicated time points by quantifying the titers of infectious virions in the supernatants with viral plaque or foci assays in the absence of the compounds. Viral replication in Calu-3 cells in the presence of indicated concentrations of compounds was determined similarly.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes. The following references correspond to the numerical denotations recited herein:
1 de Wit, E., van Doremalen, N., Falzarano, D. & Munster, V. J. SARS and MERS: recent insights into emerging coronaviruses. Nat Rev Microbiol 14, 523-534, doi: 10.1038/nrmicro.2016.81 (2016).
2 Zumla, A., Chan, J. F., Azhar, E. I., Hui, D. S. & Yuen, K. Y. Coronaviruses—drug discovery and therapeutic options. Nat Rev Drug Discov 15, 327-347, doi: 10.1038/nrd.2015.37 (2016).
3 Wu, F. et al. A new coronavirus associated with human respiratory disease in China. Nature 579, 265-269, doi: 10.1038/s41586-020-2008-3 (2020).
4 Zhou, P. et al. A pneumonia outbreak associated with a new coronavirus of probable bat origin. Nature 579, 270-273, doi: 10.1038/s41586-020-2012-7 (2020).
Li, Q. et al. Early Transmission Dynamics in Wuhan, China, of Novel Coronavirus-Infected Pneumonia. N Engl J Med 382, 1199-1207, doi: 10.1056/NEJMoa2001316 (2020).
6 Zhu, N. et al. A Novel Coronavirus from Patients with Pneumonia in China, 2019. N Engl J Med 382, 727-733, doi: 10.1056/NEJMoa2001017 (2020).
7 Cully, M. A tale of two antiviral targets—and the COVID-19 drugs that bind them. Nat Rev Drug Discov, doi: 10.1038/d41573-021-00202-8 (2021).
8 Azeez, S. A. et al. State-of-the-art tools to identify druggable protein ligand of SARS-CoV-2. Arch Med Sci 16, 497-507, doi: 10.5114/aoms.2020.94046 (2020).
9 Borgio, J. F. et al. State-of-the-art tools unveil potent drug targets amongst clinically approved drugs to inhibit helicase in SARS-CoV-2. Arch Med Sci 16, 508-518, doi: 10.5114/aoms.2020.94567 (2020).
10 Chen, Y., Liu, Q. & Guo, D. Emerging coronaviruses: Genome structure, replication, and pathogenesis. J Med Virol 92, 418-423, doi: 10.1002/jmv.25681 (2020).
11 Hegyi, A. & Ziebuhr, J. Conservation of substrate specificities among coronavirus main proteases. J Gen Virol 83, 595-599, doi: 10.1099/0022-1317-83-3-595 (2002).
12 V′Kovski, P., Kratzel, A., Steiner, S., Stalder, H. & Thiel, V. Coronavirus biology and replication: implications for SARS-CoV-2. Nat Rev Microbiol 19, 155-170, doi: 10.1038/s41579-020-00468-6 (2021).
13 Lowery, S. A., Sariol, A. & Perlman, S. Innate immune and inflammatory responses to SARS-CoV-2: Implications for COVID-19. Cell Host Microbe 29, 1052-1062, doi: 10.1016/j.chom.2021.05.004 (2021).
14 Wu, Y. et al. Main protease of SARS-CoV-2 serves as a bifunctional molecule in restricting type I interferon antiviral signaling. Signal Transduct Target Ther 5, 221, doi: 10.1038/s41392-020-00332-2 (2020).
15 Schenten, D. & Bhattacharya, D. Immunology of SARS-CoV-2 infections and vaccines. Adv Immunol 151, 49-97, doi: 10.1016/bs.ai.2021.08.002 (2021).
16 Anand, K., Ziebuhr, J., Wadhwani, P., Mesters, J. R. & Hilgenfeld, R. Coronavirus main proteinase (3CLpro) structure: basis for design of anti-SARS drugs. Science 300, 1763-1767, doi: 10.1126/science. 1085658 (2003).
17 Yang, H. et al. The crystal structures of severe acute respiratory syndrome virus main protease and its complex with an inhibitor. Proc Natl Acad Sci USA 100, 13190-13195, doi: 10.1073/pnas. 1835675100 (2003).
18 Korber, B. et al. Tracking Changes in SARS-CoV-2 Spike: Evidence that D614G Increases Infectivity of the COVID-19 Virus. Cell 182, 812-827 e819, doi: 10.1016/j.cell.2020.06.043 (2020).
19 Sterling, T. & Irwin, J. J. ZINC 15—Ligand Discovery for Everyone. J Chem Inf Model 55, 2324-2337, doi: 10.1021/acs.jcim.5b00559 (2015).
20 Schrödinger. Schrödinger Suite, Virtual Screening Workflow.
21 Pettersen, E. F. et al. UCSF Chimera—a visualization system for exploratory research and analysis. J Comput Chem 25, 1605-1612, doi: 10.1002/jcc.20084 (2004).
22 Li, Z. et al. Identify potent SARS-CoV-2 main protease inhibitors via accelerated free energy perturbation-based virtual screening of existing drugs. Proc Natl Acad Sci USA 117, 27381-27387, doi: 10.1073/pnas.2010470117 (2020).
23 Huff, S. et al. Discovery and Mechanism of SARS-CoV-2 Main Protease Inhibitors. J Med Chem, doi: 10.1021/acs.jmedchem. 1c00566 (2021).
24 Banerjee, R., Perera, L. & Tillekeratne, L. M. V. Potential SARS-CoV-2 main protease inhibitors. Drug Discov Today 26, 804-816, doi: 10.1016/j.drudis.2020.12.005 (2021).
25 Sabbah, D. A., Hajjo, R., Bardaweel, S. K. & Zhong, H. A. An Updated Review on SARS-CoV-2 Main Proteinase (M(Pro)): Protein Structure and Small-Molecule Inhibitors. Curr Top Med Chem 21, 442-460, doi: 10.2174/1568026620666201207095117 (2021).
26 Zhang, L. et al. Crystal structure of SARS-CoV-2 main protease provides a basis for design of improved alpha-ketoamide inhibitors. Science 368, 409-412, doi: 10.1126/science.abb3405 (2020).
27 Channappanavar, R. et al. IFN-I response timing relative to virus replication determines MERS coronavirus infection outcomes. J Clin Invest 129, 3625-3639, doi: 10.1172/JCI126363 (2019).
28 Channappanavar, R. et al. Dysregulated Type I Interferon and Inflammatory Monocyte-Macrophage Responses Cause Lethal Pneumonia in SARS—COV-Infected Mice. Cell Host Microbe 19, 181-193, doi: 10.1016/j.chom.2016.01.007 (2016).
29 Sastry, G. M., Adzhigirey, M., Day, T., Annabhimoju, R. & Sherman, W. Protein and ligand preparation: parameters, protocols, and influence on virtual screening enrichments. J Comput Aided Mol Des 27, 221-234, doi: 10.1007/s10822-013-9644-8 (2013).
30 Liu, Y., Ebalunode, J. O. & Briggs, J. M. Insights into the substrate binding specificity of quorum-quenching acylase PvdQ. J Mol Graph Model 88, 104-120, doi: 10.1016/j.jmgm.2019.01.006 (2019).
31 Wang, Z. et al. Comprehensive evaluation of ten docking programs on a diverse set of protein-ligand complexes: the prediction accuracy of sampling power and scoring power. Phys Chem Chem Phys 18, 12964-12975, doi: 10.1039/c6cp01555g (2016).
32 Morris, G. M. et al. AutoDock4 and AutoDockTools4: Automated docking with selective receptor flexibility. J Comput Chem 30, 2785-2791, doi: 10.1002/jcc.21256 (2009).
33 Kumari, P., Vijayan, R. & Gourinath, S. Structural analysis of EhPSP in complex with 3-phosphoglyceric acid from Entamoeba histolytica reveals a basis for its lack of phosphoglycerate mutase activity. Int J Biol Macromol 178, 1-10, doi:10.1016/j.ijbiomac.2021.02.153 (2021).
34 Corbeil, C. R., Williams, C. I. & Labute, P. Variability in docking success rates due to dataset preparation. J Comput Aided Mol Des 26, 775-786, doi: 10.1007/s10822-012-9570-1 (2012).
35 Sander, T., Freyss, J., von Korff, M. & Rufener, C. DataWarrior: an open-source program for chemistry aware data visualization and analysis. J Chem Inf Model 55, 460-473, doi: 10.1021/ci500588j (2015).
36 von Korff, M. & Sander, T. Toxicity-indicating structural patterns. J Chem Inf Model 46, 536-544, doi: 10.1021/ci050358k (2006).
37 Ghattas, M. A., Raslan, N., Sadeq, A., Al Sorkhy, M. & Atatreh, N. Druggability analysis and classification of protein tyrosine phosphatase active sites. Drug Des Devel Ther 10, 3197-3209, doi: 10.2147/DDDT.S111443 (2016).
38 Czyrski, A. Determination of the Lipophilicity of Ibuprofen, Naproxen, Ketoprofen, and Flurbiprofen with Thin-Layer Chromatography. Journal of Chemistryy 2019, 1-6, doi: 10.1155/2019/3407091 (2019).
39 Hensel, A. et al. Challenges at the Time of COVID-19: Opportunities and Innovations in Antivirals from Nature. Planta Med 86, 659-664, doi: 10.1055/a-1177-4396 (2020).
40 Abian, O. et al. Structural stability of SARS-CoV-2 3CLpro and identification of quercetin as an inhibitor by experimental screening. Int J Biol Macromol 164, 1693-1703, doi: 10.1016/j.ijbiomac.2020.07.235 (2020).
41 Muchtaridi, M., Fauzi, M., Khairul Ikram, N. K., Mohd Gazzali, A. & Wahab, H. A. Natural Flavonoids as Potential Angiotensin-Converting Enzyme 2 Inhibitors for Anti-SARS-CoV-2. Molecules 25, doi: 10.3390/molecules25173980 (2020).
42 Khalifa, S. A. M. et al. Screening for natural and derived bio-active compounds in preclinical and clinical studies: One of the frontlines of fighting the coronaviruses pandemic. Phytomedicine 85, 153311, doi:10.1016/j.phymed.2020.153311 (2021).
43 Jin, Z. et al. Structure of M(pro) from SARS-CoV-2 and discovery of its inhibitors. Nature 582, 289-293, doi: 10.1038/s41586-020-2223-y (2020).
44 Kiani, A. K. et al. Natural compounds as inhibitors of SARS-CoV-2 endocytosis: A promising approach against COVID-19. Acta Biomed 91, e2020008, doi:10.23750/abm.v91i13-S.10520 (2020).
45 Lucas, A. J., Sproston, J. L., Barton, P. & Riley, R. J. Estimating human ADME properties, pharmacokinetic parameters and likely clinical dose in drug discovery. Expert Opin Drug Discov 14, 1313-1327, doi: 10.1080/17460441.2019.1660642 (2019).

1. (canceled)

2. A pharmaceutical agent capable of inhibiting SARS-CoV-2 viral replication and/or SARS-CoV-2 related Mpro activity, wherein the pharmaceutical agent is selected from a compound recited in FIG. 14.

3. The pharmaceutical agent of claim 2, wherein the pharmaceutical agent is capable of engaging within a SARS-CoV-2 M^{pro} binding pocket characterized by one or more of the following SARS-CoV-2 M^{pro} amino acid residues: THR24, THR25, THR26, LEU27, HIE41, VAL42, CYS44, THR45, SER46, MET49, PRO52, TYR54, ASN119, PHE140, LEU141, ASN142, GLY143, SER144, CYS145, HIE163, HIE164, MET165, GLU166, LEU167, PRO168, GLY170, HIE172, PHE181, ASP187, ARG188, GLN189, THR190, ALA191, and GLN192.

4. The pharmaceutical agent of claim 3, wherein the pharmaceutical agent is selected from the group consisting of:

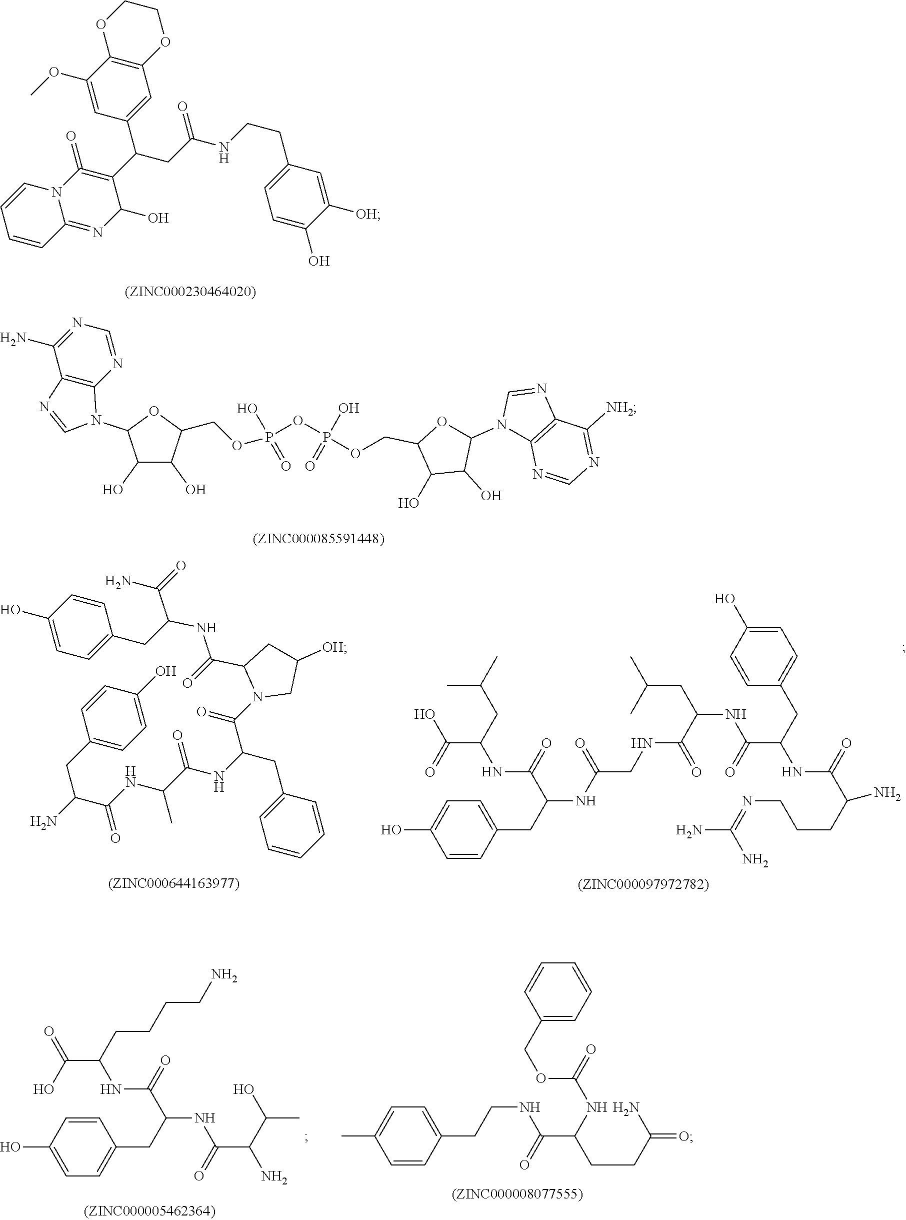

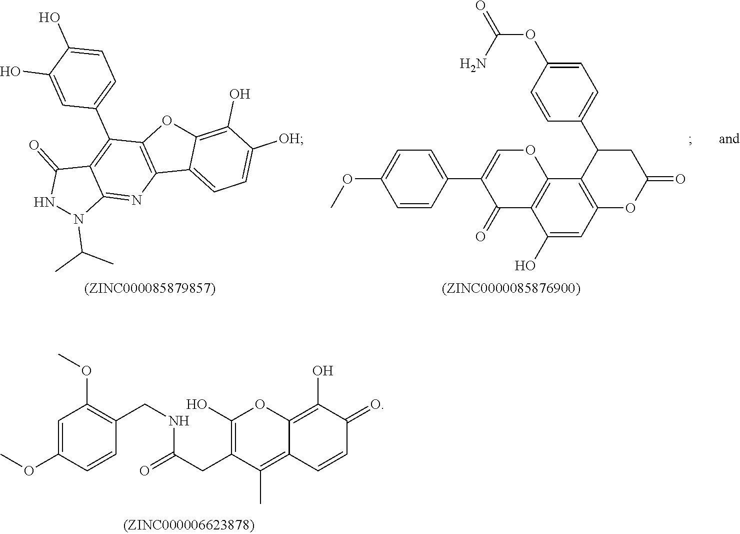 [SMILES: CC(C)n1[nH]c(=O)c2c(-c3ccc(O)c(O)c3)c3oc4c(O)c(O)ccc4c3nc21 ; COc1ccc(-c2coc3c4c(cc(O)c3c2=O)OC(=O)CC4c2ccc(OC(N)=O)cc2)cc1 ; COc1ccc(CNC(=O)Cc2c(O)oc3c(O)c(=O)ccc-3c2C)c(OC)c1]

5. The pharmaceutical agent of claim 3, wherein the pharmaceutical agent is selected from a compound recited in Table 14.

6-8. (canceled)

9. A method for treating, ameliorating and/or preventing a condition related to viral infection in a subject and/or treating, ameliorating and/or preventing symptoms related to viral infection in a subject, comprising administering to the subject a therapeutically effective amount of the pharmaceutical composition of claim 3.

10. The method of claim 9, wherein the condition related to viral infection is SARS-CoV-2 infection.

11. The method of claim 9, wherein the subject is a human subject suffering from or at risk of suffering from a condition related to SARS-CoV-2 infection.

12. (canceled)

13. The method of claim 9, wherein the administering results in suppression of M^{pro} activity.

14. The method of claim 9, wherein the administering results in production of one or more of type I interferons (IFNs), IFN-sensitive-genes (ISGs), and proinflammatory cytokines.

15. The method of claim 9, wherein the administering is oral, topical or intravenous.

16. The method of claim 9, further comprising administering to the subject one or more of hydroxychloroquine, dexamethasone, and remdesivir.

17-24. (canceled)

25. The method of claim 9, wherein the symptoms related to viral infection in a subject are one or more of fever, fatigue, dry cough, myalgias, dyspnea, acute respiratory distress syndrome, and pneumonia.

26-69. (canceled)

70. A kit comprising (1) a composition comprising a composition of claim 3, (2) a container, pack, or dispenser, and (3) instructions for administration.
